SEQUENCE LISTING (55 sequences)

SEQ ID NO: 1 - DNA, 2016 bp, Homo sapiens
  CDS (1)..(2016)
atgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtgcagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaa

SEQ ID NO: 2 - protein, 671 aa, Homo sapiens
MHHQQRMAALGTDKELSDLLDFSAMFSPPVSSGKNGPTSLASGHFTGSNVEDRSSSGSWGNGGHPSPSRNYGDGTPYDHMTSRDLGSHDNLSPPFVNSRIQSKTERGSYSSYGRESNLQGCHQQSLLGGDMDMGNPGTLSPTKPGSQYYQYSSNNPRRRPLHSSAMEVQTKKVRKVPPGLPSSVYAPSASTADYNRDSPGYPSSKPATSTFPSSFFMQDGHHSSDPWSSSSGMNQPGYAGMLGNSSHIPQSSSYCSLHPHERLSYPSHSSADINSSLPPMSTFHRSGTNHYSTSSCTPPANGTDSIMANRGSGAAGSSQTGDALGKALASIYSPDHTNNSFSSNPSTPVGSPPSLSAGTAVWSRNGGQASSSPNYEGPLHSLQSRIEDRLERLDDAIHVLRNHAVGPSTAMPGGHGDMHGIIGPSHNGAMGGLGSGYGTGLLSANRHSLMVGTHREDGVALRGSHSLLPNQVPVPQLPVQSATSPDLNPPQDPYRGMPPGLQGQSVSSGSSEIKSDDEGDENLQDTKSSEDKKLDDDKKDIKSITRSRSSNNDDEDLTPEQKAEREKERRMANNARERLRVRDINEAFKELGRMVQLHLKSDKPQTKLLILHQAVAVILSLEQQVRERNLNPKAACLKRREEEKVSSEPPPLSLAGPHPGMGDASNHMGQM

SEQ ID NO: 3 - DNA, 31 bp, Artificial Sequence
  Mini-Promoter
agagggtatataatggaagctcgacttccag

SEQ ID NO: 4 - DNA, 2204 bp, Artificial Sequence
  Expression Cassette ARN65
gaattcacctgcaagaacacctgcaagaacacctgcaagaacacctgcaagaacacctgcaagaacacctgcaagaacacctgcaagaacacctgcaagaacacctgcaagaacacctgcaagaacacctgcaagaacacctgcaagagagggtatataatggaagctcgacttccagatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtgcagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaactcgaggata

SEQ ID NO: 5 - DNA, 2063 bp, Artificial Sequence
  Expression Cassette ARN60
gaattcagagggtatataatggaagctcgacttccagatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtgcagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaactcgaggata

SEQ ID NO: 6 - DNA, 2075 bp, Artificial Sequence
  Expression Cassette ARN61
gaattcaacacctgcaagagagggtatataatggaagctcgacttccagatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtgcagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaactcgaggata

SEQ ID NO: 7 - DNA, 2099 bp, Artificial Sequence
  Expression Cassette ARN62
gaattcaacacctgcaagaacacctgcaagaacacctgcaagagagggtatataatggaagctcgacttccagatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtgcagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaactcgaggata

SEQ ID NO: 8 - DNA, 2135 bp, Artificial Sequence
  Expression Cassette ARN63
gaattcaacacctgcaagaacacctgcaagaacacctgcaagaacacctgcaagaacacctgcaagaacacctgcaagagagggtatataatggaagctcgacttccagatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtgcagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaactcgaggata

SEQ ID NO: 9 - DNA, 6294 bp, Artificial Sequence
  PAAV(exp)-SYN1>hTCF4
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 12 bp, Artificial Sequence
  microE5 box
cacctgcaagaa

SEQ ID NO: 11 - DNA, 20 bp, Artificial Sequence
  gRNA Sequence
ctttataagcccgcagttcc

SEQ ID NO: 12 - DNA, 20 bp, Artificial Sequence
  gRNA sequence
ccgcagttcccggatgtgaa

SEQ ID NO: 13 - DNA, 20 bp, Artificial Sequence
  gRNA sequence
gtcgaccagcaccgccatct

SEQ ID NO: 14 - DNA, 20 bp, Artificial Sequence
  gRNA sequence
ggtaaacagagcgcctagag

SEQ ID NO: 15 - DNA, 20 bp, Artificial Sequence
  gRNA sequence
cattcacatccgggaactgc

SEQ ID NO: 16 - DNA, 20 bp, Artificial Sequence
  gRNA sequence
acataggaaggtacgacttc

SEQ ID NO: 17 - DNA, 20 bp, Artificial Sequence
  gRNA Sequence
tttacgtaccagacatagga

SEQ ID NO: 18 - DNA, 20 bp, Artificial Sequence
  gRNA sequence
ttcctttacgtaccagacat

SEQ ID NO: 19 - DNA, 20 bp, Artificial Sequence
  gRNA sequence
ttcctatgtctggtacgtaa

SEQ ID NO: 20 - DNA, 20 bp, Artificial Sequence
  gRNA sequence
aagtcgtaccttcctatgtc

SEQ ID NO: 21 - DNA, 20 bp, Artificial Sequence
  gRNA sequence
cacgcttggcccggccatat

SEQ ID NO: 22 - DNA, 20 bp, Artificial Sequence
  gRNA sequence
ctttgcatattcaccacgct

SEQ ID NO: 23 - DNA, 20 bp, Artificial Sequence
  gRNA sequence
agcgtctgacagcagcgccg

SEQ ID NO: 24 - DNA, 20 bp, Artificial Sequence
  gRNA sequence
tcaactttgcgcagcggagc

SEQ ID NO: 25 - DNA, 20 bp, Artificial Sequence
  gRNA sequence
agacgctcaactttgcgcag

SEQ ID NO: 26 - DNA, 20 bp, Artificial Sequence
  gRNA sequence - scramble
aaatgtgagatcagagtaat

SEQ ID NO: 27 - DNA, 22 bp, Artificial Sequence
  TCF4 3b Forward Primer
gtgccgaaactacacttttgtg

SEQ ID NO: 28 - DNA, 20 bp, Artificial Sequence
  TCF4 3b Reverse Primer
acaagattatgcacctggct

SEQ ID NO: 29 - DNA, 22 bp, Artificial Sequence
  TCF4 8a Forward Primer
tggaaaggggatgcttactctc

SEQ ID NO: 30 - DNA, 20 bp, Artificial Sequence
  TCF4 8a Reverse Primer
aactctaatgacctccgcct

SEQ ID NO: 31 - DNA, 23 bp, Artificial Sequence
  TCF4 10a Forward Primer
ttaccctaaggcagttgagtgga

SEQ ID NO: 32 - DNA, 21 bp, Artificial Sequence
  TCF4 10a Reverse Primer
ttgtcaaaaatccccctcgca

SEQ ID NO: 33 - DNA, 19 bp, Artificial Sequence
  TCF4 4/5 Forward Primer
cctcctgtgagcagtggga

SEQ ID NO: 34 - DNA, 19 bp, Artificial Sequence
  TCF4 4/5 Reverse Primer
ctggacgggcttggatgtc

SEQ ID NO: 35 - DNA, 24 bp, Artificial Sequence
  TCF4 9/10 Forward Primer
gccatcttcagtctatgctccatc

SEQ ID NO: 36 - DNA, 22 bp, Artificial Sequence
  TCF4 9/10 Reverse Primer
tagggaaagtgctggttgctgg

SEQ ID NO: 37 - DNA, 21 bp, Artificial Sequence
  TCF4-A Forward Primer
ggaaagcggtctatgctccat

SEQ ID NO: 38 - DNA, 22 bp, Artificial Sequence
  TCF4-A Reverse Primer
tagggaaagtgctggttgctgg

SEQ ID NO: 39 - DNA, 23 bp, Artificial Sequence
  TCF4 12/13 Forward Primer
cttcctccgatgtccactttcca

SEQ ID NO: 40 - DNA, 21 bp, Artificial Sequence
  TCF4 12/13 Reverse Primer
cccgcttcctctatttgccat

SEQ ID NO: 41 - DNA, 22 bp, Artificial Sequence
  MPH Forward Primer
atcagggcgtgtccatgtctca

SEQ ID NO: 42 - DNA, 20 bp, Artificial Sequence
  MPH Reverse Primer
cgggtaatggcttcggggta

SEQ ID NO: 43 - DNA, 23 bp, Artificial Sequence
  dCAS9 Forward Primer
gccgacgctaatctggacaaagt

SEQ ID NO: 44 - DNA, 23 bp, Artificial Sequence
  dCas9 Reverse Primer
tggtcagggtaaacaggtggatg

SEQ ID NO: 45 - DNA, 24 bp, Artificial Sequence
  TBP Forward Primer
tgtatccacagtgaatcttggttg

SEQ ID NO: 46 - DNA, 22 bp, Artificial Sequence
  TBP Reverse Primer
ggttcgtggctctcttatcctc

SEQ ID NO: 47 - DNA, 23 bp, Artificial Sequencd
  CNTNAP2 Forward Primer
tcacacagaccaagatgagccaa

SEQ ID NO: 48 - DNA, 21 bp, Artificial Sequence
  CNTNAP2 Reverse Primer
taggaagcgaacctcgtgcca

SEQ ID NO: 49 - DNA, 22 bp, Artificial Sequence
  KCNQ1 Forward Primer
ggacaaagacaatggggtgact

SEQ ID NO: 50 - DNA, 23 bp, Artificial Sequence
  KCNQ1 Reverse Primer
gtgttgggctcttccttacagaa

SEQ ID NO: 51 - DNA, 22 bp, Artificial Sequence
  TCF4B cDNA Forward Primer
atgcatcaccaacagcgaatgg

SEQ ID NO: 52 - DNA, 32 bp, Artificial Sequence
  TCF4B cDNA Reverse Primer
atcctcgagttacatctgtcccatgtgattcg

SEQ ID NO: 53 - DNA, 59 bp, Artificial Sequence
  minP TCF4
gaattcagagggtatataatggaagctcgacttccagatgcatcaccaacagcgaatgg

SEQ ID NO: 54 - DNA, 131 bp, Artificial Sequence
  E-box-x6-minP TCF4
gaattcaacacctgcaagaacacctgcaagaacacctgcaagaacacctgcaagaacacctgcaagaacacctgcaagagagggtatataatggaagctcgacttccagatgcatcaccaacagcgaatgg

SEQ ID NO: 55 - DNA, 200 bp, Artificial Sequence
  E-box-x12-MinP TCF4
gaattcacctgcaagaacacctgcaagaacacctgcaagaacacctgcaagaacacctgcaagaacacctgcaagaacacctgcaagaacacctgcaagaacacctgcaagaacacctgcaagaacacctgcaagaacacctgcaagagagggtatataatggaagctcgacttccagatgcatcaccaacagcgaatgg

(19) United States
(12) Patent Application Publication
(10) Pub. No.: US 2023/0372542 A1
(43) Pub. Date: Nov. 23, 2023

(54) COMPOSITION AND METHODS FOR MODULATING TCF4 GENE EXPESSION AND TREATING PITT HOPKINS SYNDROME

(71) Applicant: The Regents of the University of California, Oakland, CA (US)

(72) Inventors: Alysson R. Muotri, San Diego, CA (US); Fabio Papes, Jundiai, Sao Paulo (BR)

(21) Appl. No.: 18/028,740

(22) PCT Filed: Sep. 30, 2021
(86) PCT No.: PCT/US2021/052987

§ 371 Date: Apr. 7, 2023

Related U.S. Application Data
(60) Provisional application No. 63/085,878, filed on Sep. 30, 2020.

(51) Int. Cl. A61K 48/00; C12N 15/86; C07K 14/47; A61P 25/28

(52) U.S. Cl. CPC A61K 48/0066; C12N 15/86; C07K 14/47; A61K 48/0041; A61P 25/28; C12N 2750/14143; C12N 2830/15

(57) ABSTRACT

The disclosure provides recombinant cassettes and vectors encoding TCF4 polypeptides and their use in treating neurological or neurodevelopmental disease and disorders.

### CROSS REFERENCE TO RELATED APPLICATIONS

The application claims priority under 35 U.S.C. § 119 to U.S. Provisional Application Ser. No. 63/085,878, filed Sep. 30, 2020, the disclosures of which are incorporated herein by reference.

### TECHNICAL FIELD

The disclosure relates to methods and compositions for treating neurological or neurodevelopmental diseases and disorders.

### INCORPORATION BY REFERENCE OF SEQUENCE LISTING

Accompanying this filing is a Sequence Listing entitled, “Sequence-Listing ST25” created on Sep. 30, 2021 and having 40,086 bytes of data, machine formatted on IBM-PC, MS-Windows operating system. The sequence listing is hereby incorporated by reference in its entirety for all purposes.

### BACKGROUND

Neurological and neurodevelopmental diseases and disorders including schizophrenia, autism, autism spectrum disorders are chronic and debilitating. Pitt-Hopkins syndrome (PTHS) and 18q syndrome are rare neurodevelopmental disorders characterized by symptoms including intellectual disability, failure to acquire language, deficits in motor learning, hyperventilation, epilepsy, autistic behavior, and gastrointestinal abnormalities. Certain single nucleotide polymorphisms (SNPs) in a genomic locus containing TCF4 were among the first to reach genome-wide significance in clinical genome-wide association studies (GWAS) for schizophrenia. These neuropsychiatric disorders are each characterized by prominent cognitive deficits, which suggest not only genetic overlap between these disorders but also a potentially overlapping pathophysiology.

TCF4 is a basic helix-loop-helix (bHLH) transcription factor (TF) that forms homo- or heterodimers with itself or other bHLH TFs. Dimerization of TCF4 allows for recognition of E-box binding sites (motif: CANNTG), and direct DNA binding can result in either repression or activation of transcription depending on the protein complex bound to TCF4. The TCF4 gene is highly expressed throughout the CNS during human development, but regulation of its expression and splicing is complex, as multiple alternative transcripts containing different 5′ exons and internal splicing have been identified. The genes regulated downstream of TCF4 are not well understood; this is complicated by the limited specificity of the E-box sequence, as well as context-dependent regulation of TCF4 due to heterodimerization, developmental expression and cell-type specificity.

### SUMMARY

The disclosure provides methods and compositions useful for delivery of molecules into cells.

The disclosure provides a recombinant nucleic acid construct comprising a mini-promoter operably linked to a coding sequence for a TCF4 polypeptide. In one embodiment, the nucleic acid further comprises one or more transcription factor binding motifs. In a further embodiment, the one or more transcription factor binding motifs are microE5 motifs. In still a further embodiment, the recombinant nucleic acid comprises from 1 to 15 microE5 motifs. In yet a further embodiment, the recombinant nucleic acid comprises at least 5 microE5 motifs, at least 10 microE5 motifs, or at least 12 microE5 motifs. In another or further embodiment, has a general structure of: microE5n-minipromoter-TCF4 coding sequence, wherein n is an integer in the range of 5 to 15. In yet another or further embodiment, the microE5 motif comprises the nucleotide sequence of SEQ ID NO:10. In still another or further embodiment of any of the foregoing embodiments, the TCF4 polypeptide is TCF4-B. In a further embodiment, the TCF4 polypeptide comprises an amino acid sequence having at least 85%, 90%, 95%, 98% or greater sequence identity to SEQ ID NO: 2. In yet another or further embodiment of any of the foregoing embodiments, the TCF4 coding sequence comprises a nucleotide sequence that has at least 80%, 85%, 90%, 95% or greater identity to SEQ ID NO:1. In still another embodiment the TCF4 coding sequence hybridizes under stringent conditions to a sequence consisting of SEQ ID NO:1. In still another embodiment, the mini-promoter of any of the foregoing embodiments comprises a core promoter. In a further embodiment, the mini-promoter comprises a nucleotide sequence that has at least 70%, 80%, 90%, or greater sequence identity to SEQ ID NO:3. In another embodiment, the mini-promoter comprises the nucleotide sequence of SEQ ID NO:3, optionally with from 1 to 5 nucleotide modifications independently selected from deletions, insertions, and substitutions. In still another or further embodiment of any of the foregoing embodiments, the nucleic acid construct comprises a nucleotide sequence that is at least 80% identical to any one of SEQ ID Nos: 4, 5, 6, 7 or 8.

The disclosure also provides a vector comprising a recombinant nucleic acid of any of the foregoing. In a further embodiment, the vector is a viral vector. In still a further embodiment, the viral vector is a retroviral vector. In yet a further embodiment, the vector is an adeno-associated virus (AAV) vector, lentiviral vector or gamma-retroviral vector. In still a further embodiment, the vector is an AAV9 vector.

The disclosure also provides a recombinant cell comprising the recombinant nucleic acid of the disclosure or the vector of the disclosure.

The disclosure also provides a pharmaceutical composition comprising a vector of the disclosure.

The disclosure also provides a method of treating a neurological or neurodevelopmental disease or disorder in a subject, comprising transforming a neuron of the subject with a recombinant nucleic acid of the disclosure or administering a vector of the disclosure, or administering the pharmaceutical composition of the disclosure to the subject. In still another embodiment, the neurological or neurodevelopmental disease or disorder is Pitt-Hopkins Syndrome, schizophrenia, autism, autism spectrum disorder, or 18q syndrome. In still another embodiment, the neurological or neurodevelopmental disease or disorder is Pitt-Hopkins Syndrome, and is associated with TCF4 haploinsufficiency. In still another or further embodiment, the subject has one or more single nucleotide polymorphisms in a TCF4 gene. In yet still another or further embodiment, the subject has a chromosomal deletion including at least a portion of a TCF4 gene. In a further embodiment, the subject has a complete deletion of a TCF4 gene. In still another embodiment, the subject has a chromosomal translocation comprising at least a portion of a TCF4 gene. In another embodiment, the subject has a translocation, frameshift, or non-sense mutation in a TCF4 gene. In another or further embodiment, the subject is an infant or pediatric subject. In a further embodiment, the subject is about 16 years of age or less. In still another embodiment, the subject is about 12 years of age or less. In yet another embodiment, the subject is about 8 years of age or less, about 5 years of age or less, or about 2 years of age or less. In another embodiment, the subject is an adult subject.

The disclosure also provides a method of treating a neurological or neurodevelopmental disease or disorder related to TCF4 haploinsuffiency in a subject, comprising increasing expression of one or more of SOX3 and SOX4 in neurons of said subject. In another embodiment, expression of SOX3 and/or SOX4 are increased by introducing a recombinant nucleic acid expressing TCF4-B polypeptide to said neurons. In a further embodiment, the recombinant nucleic acid comprises a mini-promoter operably linked to a coding sequence for the TCF4-B polypeptide. In still a further embodiment, the recombinant nucleic acid further comprises one or more transcription factor binding motifs. In a further embodiment, the one or more transcription factor binding motifs are microE5 motifs. In still a further embodiment, the recombinant nucleic acid comprises from 1 to 15 microE5 motifs. In yet another or further embodiment, the recombinant nucleic acid comprises at least 5, at least 10, or at least 12 microE5 motifs. In another embodiment, the nucleic acids has a general structure of: microE5n-minipromoter-TCF4 coding sequence, wherein n is an integer in the range of 5 to 15. In still another or further embodiment, the microE5 motif comprises the nucleotide sequence of SEQ ID NO:10. In yet another or further embodiment, the recombinant nucleic acid expressing TCF4-B polypeptide is delivered to said subject with a viral vector. In a further embodiment, the viral vector is a retroviral vector. In still a further embodiment, the vector is an adeno-associated virus (AAV) vector, lentiviral vector or gamma-retroviral vector. In a further embodiment, the vector is an AAV9 vector. In another or further embodiment of any of the foregoing embodiments, the neurological or neurodevelopmental disease or disorder is Pitt-Hopkins Syndrome, schizophrenia, autism, autism spectrum disorder, or 18q syndrome. In a further embodiment, the neurological or neurodevelopmental disease or disorder is Pitt-Hopkins Syndrome. In still another or further embodiment of any of the foregoing embodiments, the subject has one or more single nucleotide polymorphisms in a TCF4 gene. In still another or further embodiment of any of the foregoing embodiments, the subject has a chromosomal deletion including at least a portion of a TCF4 gene. In still another or further embodiment of any of the foregoing embodiments, the subject has a complete deletion of a TCF4 gene. In still another or further embodiment of any of the foregoing embodiments, the subject has a chromosomal translocation comprising at least a portion of a TCF4 gene. In still another or further embodiment of any of the foregoing embodiments, the subject has a translocation, frameshift, or non-sense mutation in a TCF4 gene. In still another or further embodiment of any of the foregoing embodiments, the subject is an infant or pediatric subject. In a further embodiment, the subject is about 16 years of age or less, about 12 years of age or less, about 8 years of age or less, about 5 years of age or less or about 2 year of age or less. In another embodiment, the subject is an adult subject.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1A-B shows levels of expression of TCF4 in neural progenitor cells. (A) Each bar represents the TPM expression abundance (transcripts per million) in RNA sequencing libraries produced from PTHS individuals (red bars) and healthy controls, which are parents of afflicted children (black bars). The transcript isoforms on the horizontal axis were named according to the Ensembl database entry for the human TCF4 gene. (B) Correspondence between each transcript variant and the respective protein isoform, named according to Sepp et al., 2011 (Functional Diversity of Human Basic Helix-Loop-Helix Transcription Factor TCF4 Isoforms Generated by Alternative 59 Exon Usage and Splicing. PLoS ONE 6(7): e22138. doi:10.1371/journal.pone.0022138).

FIG. 2A-C shows constructs and validation of a DNA cassette for over-expression of TCF4. (A) Schematic diagrams representing expression constructs where the sequence of the TCF4-B cDNA is under the control of a minimal promoter (minP) preceded by varying numbers of pE5 boxes. (B) Western blot analysis for TCF4 protein in HEK293 cells expressing constructs depicted in A, along with empty vector and ‘no DNA’ controls. Representative blot of n=3 replicates. β-actin was used as loading control. (C) DNA sequence of the regulatory elements preceding the TCF4-B coding sequence in one example of DNA construct generated in the study—which includes 12 μE5 boxes and minimal promoter minP.

FIG. 3A-B shows increase in expression of TCF4 and one of its target genes after introduction of DNA constructs into patient-derived neural progenitor cells. (A) Relative expression levels of TCF4 gene in control cell lines (orange bars) and PTHS-derived cell lines (blue bars), after transduction with lentiviral particles (viral vectors) containing 12 or 6 μE5 boxes, according to details outlined in FIG. 2C. Two patient lines and controls were used for the experiment. n=3 biological replicates per treatment; 3 technical replicates per biological replicate. (B) Similar to A, but for GADD45G gene, one of the known targets of TCF4 in human progenitor cells. Bars representing control lines transduced with viral particles are grayed out to highlight the comparison between orange bars (normal levels of GADD45G expression) and blue bars (expression in diseased cells before and after genetic manipulation).

FIG. 4A-E provide exemplary TCF4 cassettes of the disclosure (SEQ ID NO:4-8).

FIG. 5A-E shows PTHS organoids display aberrant development and altered content of neural progenitors and cortical neurons. (A) Bright-field microscopy images of pallial cortical organoids (CtO) derived from controls (parents) and PTHS individuals over 4 weeks of culture in vitro. (B) Left: CtO size distribution at 4 weeks of culture in vitro, for 4 parent-child pairs (see Table 1 for a description of subjects involved in this study). Right: Mean CtO size at 4 weeks. N=4 subjects per group (indicated by different symbols according to key in Table 1), 12-30 organoids per subject. (C) Microscopy images of control and PTHS subpallial organoids (sPOs) over 4 weeks of culture in vitro. Due to the organoids' large sizes, images at 4 weeks were taken directly from a 3.5 cm diameter plate. (D) Quantification of SOX2+ cell content at two stages of development. N=4 subjects (symbols), 3 batches per subject, 6 organoids per batch, 4 random 100×100 μm regions of interest (ROI) per organoid. See FIG. 12F for quantification of SOX2+ cells in sPOs. (E) Quantification of the content of cortical neurons expressing CTIP2 at two stages of CtO development. N=4 subjects (symbols), 3 batches per subject, 6 organoids per batch, 4 random ROIs per organoid. See FIG. 12G for quantification of SATB2+ cells in CtOs.

FIG. 6A-J shows PTHS organoids have increased percentage of neural progenitors and decreased percentage of excitatory cortical neurons and inhibitory interneurons. (A) Uniform Manifold Approximation and Projection (UMAP) bidimensional reduction of single cell RNA-Seq transcriptomic profiling of CtOs and sPOs from parental controls and PTHS organoids. Color code represents 6 annotated subpopulations: Pr-Glut, neural progenitor cells in glutamatergic lineage; IP-Glut, intermediate progenitors in glutamatergic ineage; N-Glut, glutamatergic neurons; Pr-GABA, neural progenitors in inhibitory lineage; IP-GABA, intermediate progenitors in inhibitory lineage; N-GABA, GABAergic interneurons (see also FIG. 13A). Other cell types are not shown. (B) Trajectory analysis indicating the existence of separate cell differentiation lineages in CtOs and sPOs. Colormap represents progression along the pseudotime in each lineage (glutamatergic or GABAergic). (C) Comparison of content of different cell types between parent and PTHS CtOs. Color code is the same as in A. Black dots represent cells in other populations not depicted in A. (D) Quantification of percentage of cell types in each subpopulation in CtOs (color code is the same as in A). (E) Left: SOX2 expression levels in Pr-Glut subpopulation in CtOs; each dot represents a single cell. Right: Percentages of cells expressing SOX2 (above threshold equal to 40% of the mean). (F) Comparison of cell populations between parent and PTHS sPOs. (G) Quantification of the percentage of cell types in each subpopulation in sPOs. (H) Left: SOX2 expression levels in Pr-GABA subpopulation in sPOs. Right: Percentages of SOX2+ cells. (I,J) Left: Expression of CTIP2 and SATB2 (I) or GAD2 (J) in N-Glut subpopulations in CtOs (I) or N-GABA subpopulations in sPOs (J). Right: Severe reduction in percentages of neuronal subtypes in PTHS CtOs and sPOs (expression above threshold corresponding to 40% of the mean).

FIG. 7A-H shows PTHS neurons exhibit abnormal electrophysiological properties and gene expression program. (A) Left: CtOs seeded on multi-electrode array (MEA) plate. Right: Mean firing rate in CtOs. N=4 subjects per group (symbols), 3 independent replicates per subject. (B) iPSC-derived neurons in bidimensional culture conditions, immunostained for MAP2 (white). (C) Comparison of neurite length and soma area between parent and PTHS-derived neurons. Medians are indicated by the colored lines. N=30-80 neurons per line (dots), from subjects in parent-child pairs #1 and #4. (D) Patch-clamp electrophysiological interrogation of iPSC-derived neurons, showing reduction in spike rate (top) and intrinsic excitability (bottom) in PTHS neurons. N=10 (parent) or 9 (PTHS) neurons. (E) Comparison of sodium and potassium current measurements between PTHS (blue line) and parental control (orange line). N=10 (parent) or 9 (PTHS) neurons. (F) Left: Expression of FOS in N-Glut neurons in CtOs; violin plots represent distribution of gene expression in each population; N=1401 (parent) and 380 (PTHS) cells. Right: Percentages of FOS+ cells. (G) Relative expression (RT-qPCR) of selected neuronal genes in iPSC-derived neurons. N=4 subjects per group (symbols), 3 independent replicates per subject, 2 technical replicates per sample. In control groups, mean gene expression was normalized to 1. STMN2, stathmin 2; TAC1, tachykinin precursor 1; CNTN2, contactin 1; INA, internexin neuronal intermediate filament protein alpha; ADCYAP1, adenylate cyclase activating polypeptide 1; SYT13, synaptotagmin 13; and SLC17A6, vesicular glutamate transporter 2 (VGLUT2). (H) Expression of the same genes as in G in single cell transcriptomic data from N-GABA neurons in sPOs. Sample sizes are the same as in F. Bar graphs represent mean+SEM. n.s., not statistically significantly different, *p<0.05, **p<0.01, ***p<0.001; Kruskal-Wallis H test (F and H), Welch's t test (A), ANOVA with Geisser-Greenhouse correction for repeated measures followed by LSD post-hoc test (C), or ANOVA followed by HSD post-hoc test (D and E). Scale bars are 100 μm. In F and H, statistical comparisons are between means of gene expression for each gene.

FIG. 8A-J shows PTHS neural progenitors proliferate at a lower rate. (A) Left, Immunostaining of CtOs at week 2 for SOX2 and MAP2. Arrowheads mark rosette examples. Middle, Graph showing number of rosettes in parent and PTHS organoids at week 2. Right, Density of SOX2+ cells in organoids. N=4 subjects per group (symbols), 3 technical replicates. (B) Left: Growth curve for NPCs; lines represent mean number of cells; N=3 independent replicates per time point (circles), 3 technical replicates. Right: Relative live cell count for neural progenitors after 4 days in culture (starting number of cells=100,000). N=4 subjects per group (symbols), 3 independent biological replicates, 3 technical replicates. (C) Quantification of Annexin V-positive (apoptotic) cells in NPCs. N=4 subjects per group (symbols), 3 independent replicates per subject. (D) Live cell count in NPC proliferation assay. N=4 subjects per group (symbols), 3 independent replicates per subject. (E) Left: Flow cytometry assessment of EdU-positive (dividing) NPCs. Right: Percentage of EdU+ cells; N=3 subjects per group (symbols), 3 independent replicates per subject, 6 technical replicates. (F) Morphological abnormality in PTHS NPCs showing flat enlarged cells (arrowheads). (G) Left: Staining for senescence-associated β-galactosidase (SA-β-gal) activity (green fluorescence) in NPCs. Quantification is shown on the right. N=4 subjects per group (symbols), 3 independent replicates per subject. (H) Relative expression of CDKN2A (cyclin-dependent kinase inhibitor 2A; left) and LMNB1 (lamin B1; right) in NPCs. N=4 subjects per group (symbols), 3 independent replicates per subject, 2 technical replicates. (I) Relative expression of CDKN2A in post-mortem PTHS cortex sample. (J) Quantification of p16^{INX4a}+ (left) and apoptotic (Cleave Caspase 3, CC3+; right) cells in CtOs. N=4 subjects per group (symbols), 2 batches, 6 organoids per batch, 4 100×100 μm ROIs per organoid. All bar graphs represent mean+SEM. n.s.=not statistically significantly different. *p<0.05, **p<0.01, ***p<0.001; ANOVA (in left panel in C) or Welch's t-test in remaining comparisons. In panel H, mean gene expression in controls was normalized to 1. DAPI nuclear staining in blue. Scale bars are 100 μm.

FIG. 9A-K shows manipulation of Wnt signaling pathway rescues abnormal proliferation of PTHS neural progenitors. (A) Ratio of expression abundances (transcripts per million, TPM) for Wnt signaling pathway genes between parent and PTHS NPCs. N=4 parent-child pairs (symbols). (B) Relative expression of selected Wnt signaling genes in NPCs. N=4 subjects per group (symbols), 3 independent replicates per subject, 2 technical replicates. (C) Reduced Wnt signaling activity in PTHS NPCs (TOP-Flash assay). N=4 subjects per group (symbols), 3 independent replicates per subject. Mean activity (arbitrary units) was normalized to 1 in ‘parents’ group. (D) Relative expression of selected Wnt genes in post-mortem PTHS cortex sample. (E) Treatment of control NPCs with Wnt pathway antagonists DKK-1 and ICG-001 (yellow bars) phenocopies proliferation deficit in PTHS progenitors. N=3 replicates per group (dots); cells from parent-child pair #4 in Table 1. (F) ICG-001 treatment phenocopies low neural progenitor content (SOX2) of PTHS organoids. N=3 independent expts (dots); 12 assessed organoids per experiment in each group; 4 random 100×100 μm ROIs per organoid. (G) Live cell count showing treatment of NPCs with Wnt pathway agonist CHIR99021. N=3 subjects per group (symbols), 3 independent replicates per subject, 3 technical replicates. (H) EdU proliferation assay in NPCs treated with CHIR99021. Left graph represents data for parent/patient pair #4, and right graph shows data for pair #1. N=3 technical replicates. (I) Quantification of p16^{INK4a}+ (senescent) cells in NPCs treated with CHIR99021. Data is for pair #4 (see also FIG. 16E for pair #1). (J) CHIR99021 rescues expression of several progenitor genes in treated PTHS NPCs. N=4 subjects per group (symbols), 3 independent replicates per subject, 2 technical replicates. (K) Quantification of SOX2+ cells after PTHS CtO treatment with CHIR99021. N=3 expts (dots); 6 organoids per experiment in each group; 4 random 100×100 μm ROIs per organoid. Bar graphs represent mean+SEM. n.s., not statistically significantly different; **p<0.01, ***p<0.001; Welch's t-test (in B, C and D) or ANOVA was performed for the remaining panel. In B, D and J, mean control gene expression is 1. In K, statistical comparison is between means of PTHS+CHIR and PTHS+DMSO (control) groups. Scale bars are 100 μm.

FIG. 10A-L shows Mechanistic involvement of SOX genes in PTHS cellular pathophysiology. (A) Ratio of expression abundances (TPM) for several SOX genes in NPCs. N=4 parent-child pairs (symbols). Brackets above bars show SOX gene classes. (B) Top: SOX3 expression abundance (TPM) in NPCs. Bottom: SOX3 relative expression determined via qPCR. N=4 subjects per group (symbols), 3 independent replicates per subject, 2 technical replicates. (C) SOX3 is downregulated after shRNA-mediated TCF4 knockdown in NPCs (cells from parent #4 in Table 1). N=3 independent replicates per group (dots), 2 technical replicates. (D) SOX3 relative expression in post-mortem PTHS cortex sample. (E) Immunostaining for SOX3 in post-mortem PTHS sample (two ROI shown per genotype). (F) Treatment of PTHS NPCs with CHIR99021 rescues aberrant SOX3 expression. N=4 subjects per group (symbols), 3 independent replicates per subject, 2 technical replicates. (G) shRNA-mediated SOX3 knockdown reduces progenitor proliferation. N=3 independent replicates (dots) per group, 3 technical replicates. (H) SOX4 expression is reduced in PTHS NPCs. Top: Expression abundance (in TPM). Bottom: Relative expression. N=4 subjects per group (symbols), 3 independent replicates per subject, 2 technical replicates. (I) SOX4 expression is reduced in intermediate progenitors (IP-Glut) and neurons (N-Glut) in PTHS CtOs. N=717 (parent) and 382 (PTHS IP-Glut cells, or 1401 (parent) and 380 (PTHS)N-Glut neurons. (J) Ratio between neurons (MAP2+) and NPCs (SOX2+) as a proxy for neuronal differentiation rate. N=4 subjects per group (symbols). (K) Ratio between MAP2+ and SOX2+ after SOX4 knockdown. N=3-4 ROIs per genotype; cells are from parent/child pairs #1 and #4. (L) Ratio between MAP2 and SOX2 gene expression levels after SOX4 knockdown. N=4 biological replicates; cells are from parent/child pairs #4. Bar graphs represent mean+SEM. *p<0.05; **p<0.01; ***p<0.001; Kruskal-Wallis H test (I), or ANOVA (F and G) and Welch's t-test in the remaining panels. Scale bar is 100 μm. DAPI nuclear staining in blue.

FIG. 11A-H shows reversal of abnormal phenotypes in PTHS organoids subjected to genetic correction of TCF4 expression. (A) Schematic representation of CRISPR-based trans-epigenetic correction of TCF4 expression using constructs for guide RNA (gRNA), transcriptional activation module MPH and dead Cas9. (B) Top: Virus application regimen. Bottom: Brightfield images of PTHS brain organoids subjected to correction of TCF4 expression (PTHS+TCF4 gRNA), compared with controls transduced with scrambled gRNA (scr gRNA). (C) Fluorescence microscopy images of transduced organoids after immunostaining for TCF4; C′: clustered TCF4+ cells (arrowhead) in aberrant outgrowth. (D) Transduced organoids stained for MAP2 and SOX2, at two developmental time points. Arrowheads: aberrant outgrowths in scr gRNA PTHS. High mag insets: clustered abnormally shaped MAP2+ cells in organoid outgrowths. (E) Over-expression construct showing placement of the TCF4-B coding sequence under the control of a synthetic promoter composed of minimal promoter minP and varying number of TCF4 binding sites (□E5 boxes). (F) Top: Virus application regimen. Bottom: Microscopy images showing general morphology of 6 weeks-old organoids transduced with TCF4 OE vector or an empty vector (ctrl) (top line), immunostaining for SOX2 and MAP2 (middle line), and for CTIP2 (bottom line). Arrowhead, neural rosette. (G) Left, Raster plot showing electrical activity of transduced 2-to-3 months-old organoids subjected to multi-electrode array (MEA) analysis. Each row represents an electrode. Vertical red rectangles represent events of bursts of electrical activity that happen at the network level (network bursts). Right, Quantification of mean firing rate (top right) in transduced organoids over time (see also FIG. 18L for raster plots), and number of network bursts (bottom). (H) Top: Virus application regimen. Bottom: Immunostaining for TCF4 (top line), SOX2 and MAP2 (middle) and CTIP2 (bottom). Arrowhead, neural rosette. Bar graphs represent mean+SEM. n.s., not statistically significantly different, *p<0.05; **p<0.01; ***p<0.001; Welch's t-test at each time point in G. Scale bars are 100 μm. DAPI nuclear staining in blue.

FIG. 12A-M shows PTHS iPSCs exhibit normal growth rate and can be differentiated into neurons. (A) Structure of the TCF4 exons (numbers on top of each rectangle) in different patients. White rectangles symbolize missing exons due to partial or whole gene deletion. Rectangles with thick borders represent the coding sequence in each case. AD1 to AD3, transcriptional activation domains; bHLH, basic helix-loop-helix DNA binding domain. Exons 1 and 2 are shown but they are not part of the main transcript for the TCF4 gene, called TCF4-B. Details on the types of mutation carried by each patient are given on the right (see also Table 1 for further information). (B) Example of digital karyotyping by SNP mapping via chip hybridization, on sample from patient PTHS #2 (Table 1), showing large deletion on chromosome 18 (asterisk). Numbers on the left represent chromosomes. The y axis in each chromosome graph represents the log R ratio for individual hybridized chip probes (dots). (C) Comparison of iPSC colony growth rate (in days to reach size required for passaging, which is 2 mm) between parents (controls), PTHS, and a control iPSC line derived from a subject not involved in the study (WT83). N=5 subjects (symbols). Each measurement is the median from 5 independent plates. (D) Representative bright-field microscopy image showing iPSC-derived neurons in culture. Notice the formation of bundles of progenitors and neuronal cell bodies in both the parent and PTHS groups. (E) Fluorescence microscopy images of cultures in D stained for MAP2 (red) and SOX2 (green), showing ability of iPSCs to differentiate into NPCs (SOX2+) and neurons (MAP2+) in both groups. (F) Left, Bright-field images of PTHS and parental control CtOs derived from iPSCs of two different batches (clones). Middle, Organoid size distribution. Right, Immunostaining for SOX2 and MAP2. Arrowheads point small rosettes in the patient lines. (G) Percentage of SOX2+ cells in 4 and 10 weeks-old CtOs of parent and PTHS genotypes. N=4 subjects (symbols). (H) Fluorescence microscopy images of parent and PTHS sPOs after immunostaining for NPC marker SOX2 (green) and neuronal marker MAP2 (red), at 6 weeks in vitro. (I) Quantification of the density of SOX2+ cells in parent and PTHS sPOs at 6 weeks in vitro. N=4 subjects (symbols), 2 batches per subject, 6 organoids per batch, 4 random 100×100 □m regions of interest (ROI) per organoid. (J) Quantification of the density of cortical neurons expressing SATB2 in parent and PTHS CtOs at two stages of development in vitro. N=4 subjects (symbols), 3 batches per subject, 6 organoids per batch, 4 random ROIs per organoid. (K) Relative expression of neural markers in post-mortem PTHS cortex sample. (L) Quantification of percentage of CTIP2+ cells in post-mortem sample. (M) Quantification of vGLUT1 and GAD65/67 expression, as judged from number of pixels above threshold intensity per unit area in PTHS and control CtOs and sPOs. N=6 sections per condition (circles), 4 ROIs per section. Bar graphs represent mean+SEM. n.s., not statistically significantly different, **p<0.01, ***p<0.001; two-sample Welch's t test (D, G, I and K), one-way ANOVA followed by Tukey-Kramer's HSD post-hoc test against respective parent group (G), or Wilcoxon-Mann-Whitney U test (M). Scale bars are 100 μm.

FIG. 13A-I shows annotation of subpopulations in single cell RNA-Seq experiments and associated controls. (A) Dot plot showing expression of selected marker genes in the six subpopulations of cells depicted in FIG. 6A. Pr-Glut, neural progenitor cells in glutamatergic lineage; IP-Glut, intermediate progenitors in glutamatergic ineage; N-Glut, glutamatergic neurons; Pr-GABA, neural progenitors in inhibitory lineage; IP-GABA, intermediate progenitors in inhibitory lineage; N-GABA, GABAergic interneurons. ‘Others’ represent a heterogeneous group of cells not included in the previous six categories. Dot sizes are the percentages of cells in each subpopulation that have detectable expression for the corresponding gene. (B) Violin plots for marker genes shown in A, displaying range of expression in the six analyzed subpopulations of cells (and ‘Others’). Color code is the same as in FIG. 2A. For GRIN2, GAD1, and GAD2, the medians were low and therefore the expression in each cell is represented as a dot. (C) Single cell RNA-Seq quality control data. Violin plots represent the number of read counts, number of detected genes (features), and percentage of mitochondrial genes (mtRNA) in the subpopulations listed in A. Color code is the same as in FIG. 6A and ‘Others’ are shown in black. (D) UMAP showing expression of TCF4, neural lineage markers SOX2 and MAP2, mesoderm markers MIXL1 and TBXT (Brachyury), and endoderm markers CFTR and SOX17. Intensity of purple indicates relative expression level. (E) Characterization of unassigned cells in ‘Others’ subpopulation. Top left, Percentages of cells in ‘Others’ and high mitochondrial RNA content subpopulations, in parent and PTHS CtOs and sPOs. Top right, Expression levels of neural lineage markers SOX2 and MAP2 in CtOs and sPOs in parent (orange) and PTHS (blue) organoids. Bottom, Expression levels of mesoderm and endoderm markers in D, in CtOs and sPOs of parent (left in each group) and PTHS (right in each group) genotypes. (F) Left, UMAP showing expression of astrocytic markers S100B and ALDH1L1 in parent and PTHS CtOs. Right, Expression level comparison for the same genes in parent and PTHS CtOs. (G) Controls showing reproducibility of CtO generation in independent batches (replicates #1 and #2; left) and robustness of the single cell RNA-Seq analyses, which detect similar percentages of each cell type in independent batches of parent-derived organoids (right). Color code is the same as in FIG. 6A. (H, I) Left: Comparison between parent and PTHS CtOs in terms of expression for genes coding for markers of cortical neuronal subtypes TBR1 (H) and CUX (I) in N-Glut subpopulation. Right: Severe reduction in percentages of neuronal subtypes in PTHS CtOs (cells expressing each marker gene above threshold corresponding to 40% of the corresponding mean). n.s., not statistically significantly different, Wilcoxon-Mann-Whitney U test (F).

FIG. 14A-H shows supporting data for investigation of neurons in 2D culture. (A) Time course of mean firing rate in CtOs subjected to multi-electrode array (MEA) assay, showing comparison between parent (orange) and PTHS (blue) organoids. Each patient is represented with a different symbol, as indicated in Table 1. The red lines and error bars represent the mean across all subjects over time. N=4 subjects (symbols), 3 independent replicates per subject. (B) Representative fluorescence microscopy image showing expression of TCF4 protein (green) in neurons (MAP2 labeling is shown in red) being differentiated in 2D culture from parent-derived iPSCs. (C) Relative TCF4 expression levels (RT-qPCR) between iPSC-derived PTHS and parent neuronal cultures. N=4 subjects (symbols), 3 independent replicates per subject, 2 technical replicates per sample. Each PTHS sample is compared to the respective parent (expression normalized to 1). (D) Comparison of membrane capacitance between PTHS (blue circles) and parental control neurons (circles) in 2D culture via patch-clamp electrophysiological analysis. (E) Interrogation of sodium (top) and potassium (bottom) current densities, comparing PTHS (blue line) and control (orange line) neurons. N=10 (parent) or 9 (PTHS) neurons. (F) Heat map showing the expression levels for the 20,000 most highly expressed genes in RNA-Seq libraries of neurons from one parent and respective PTHS child (PTHS #4 in Table 1). The numbers of differentially expressed (DE) genes in the parent-child comparison are shown above the plot. (G) Dot plot results for Gene Ontology—Biological Processes (top) and Pathway analysis (bottom), for down-regulated DE genes in D. For each analysis, the top 10 categories in terms of adjusted p-value are shown. Dot size represents number of DE genes that fall into each classification category, dot color is the adjusted p-value, and the x-axis represents the percentage of genes in each category that are DE expressed genes in the RNA-Seq libraries. Notice the presence of genes involved in glutamatergic and GABAergic transmission. (H) MA plot for genes expressed in control and PTHS neurons. Gray dots are genes that are not statistically significantly differentially expressed between control and PTHS. Blue and red dots are statistically significant DE genes. Red dots represent genes coding for sodium or potassium channels with a log₂ fold change superior to 4 (dashed lines). Bar graphs represent mean+SEM. *p<0.05, **p<0.01, ***p<0.001, Welch's t-test (A and D) or ANOVA followed by HSD post-hoc test (E). Scale bar is 100 μm.

FIG. 15A-N shows expression analysis in neural progenitor cells. (A) Validation of expression of several NPC markers in iPSC-derived neural progenitor cells from parents and PTHS subject, as judged from TPM expression abundance in RNA-Seq libraries. N=4 subjects per group (symbols), 3 independent replicates per subject. (B) Relative TCF4 expression levels (RT-qPCR) in iPSCs and iPSC-derived NPCs and neuronal cultures, as well expression in a non-neural cell line (HEK293T). N=4 independent replicates per group, 2 technical replicates per sample. Mean expression in neural progenitor group was normalized to 1. (C) Representative fluorescence microscopy image of NPCs in 2D culture after immunostaining for TCF4 (red) and NPC marker Nestin (green). Higher magnification image in inset. (D) Representative fluorescence microscopy image showing abundant expression of TCF4 (red) in NPCs of rosettes in control (parental) CtOs. (E) Relative expression (RT-qPCR) of TCF4 in NPCs derived from parents and respective PTHS children. N=5 subjects per group (symbols; subjects PTHS #1 to #5 in Table 1), 3 independent replicates per subject, 2 technical replicates per sample. Notice that for one PTHS subject (circle symbol) expression is not significantly diminished, which was not expected since this patient (PTHS #4) carries a point mutation that is not expected to affect transcript abundance. All the others have mutations expected to decrease transcript content (non-sense mutation, frameshift mutation, whole gene deletion, and translocation). The mean expression for each PTHS subject is shown relative to its respective parent (all parent means normalized to 1). (F) Relative expression of GADD45G in NPCs from parents and respective PTHS children. N=5 subjects per group (symbols), 3 independent replicates per subject, 2 technical replicates per sample. (G) Higher magnification fluorescence images showing diminished expression of TCF4 in PTHS (patient PTHS #2) and possible mis-localization of the TCF4 protein outside the cell nuclei (arrowhead). (H) Ratio between the expression of transcriptomic markers of replicative senescence between PTHS and control samples. Cells are from parent-child pair #4 (Table 1). For each gene, the mean at each passage was determined from 3 independent biological samples. Each line connects expression for a certain gene at early and late passage conditions. Marker genes are separated according to class (downregulated or upregulated in senescent cells). Notice the more pronounced mis-regulation in late passage conditions. Similar results were obtained for parent-child pair #1. (I) Quantification of the percentages of cells expressing senescence marker p16^{INK4a} cells that also express neural lineage marker Nestin, progenitor marker SOX2, mesoderm marker Brachyury and endoderm marker SOX17. For SOX2, some cells exhibit strong staining and others are weakly stained. (J) Left, High magnification images of CtOs stained for SOX2, MAP2 and p16^{INK4a}, followed by quantification of cells co-expressing p16^{INK4a} with SOX2 or MAP2 (right). (K) Left: shRNA-mediated TCF4 knockdown reduces NPC proliferation. N=3 independent replicates (dots) per group, 3 technical replicates per sample. Initial seeding density is 1×10⁵ cells. Right: Quantification of percentage of EdU-positive NPCs in the same groups; N=3 independent replicates per group (dots). (L) shRNA-mediated TCF4 knockdown leads to decreased expression of TCF4 and TCF4 downstream target gene GADD45G, as well as increased expression of senescence marker CDKN2A. N=3 independent replicates per group (dots), 2 technical replicates per sample. (M) Heat map showing the expression levels for the 20,000 most highly expressed genes in RNA-Seq libraries of NPCs from 2 parents and respective PTHS children, as an example. The numbers of differentially expressed (DE) genes in the intersection among all four parent-child comparison are shown above the plot. (N) Dot plot results for Gene Ontology—Biological Processes (top) and Pathway analysis (bottom), for down-regulated DE genes listed in M resulting from the intersection among all 4 parent-child pairs. For each analysis, the top 10 categories in terms of adjusted p-value are shown. Dot size represents number of DE genes that fall into each classification category, dot color is the adjusted p-value, and the x-axis represents the percentage of genes in each category that are DE expressed genes in the RNA-Seq libraries. Notice the presence of downregulated genes in the Wnt signaling pathway. Bar graphs represent mean+SEM. *p<0.05, **p<0.01, ***p<0.001; two-sample Welch's t-test assuming unequal variances (E, F, and K) or ANOVA followed by Tukey-Kramer's HSD post-hoc test (K). In L, mean gene expression was normalized to 1 in each parent+control shRNA group. Scale bars are 100 μm.

FIG. 16A-N shows additional controls for Wnt signaling manipulation in organoids. (A) Expression abundances for selected genes in the Wnt signaling pathway, comparing parent (orange) and PTHS (blue) NPCs. N=4 subjects per group (symbols), 3 independent replicates per subject. (B) Treatment of parent-derived NPCs with DKK-1 increases expression of senescence marker CDKN2A. N=4 biological replicates (symbols). (C) Treatment of control CtOs with Wnt pathway antagonist ICG-001 (yellow bar) phenocopies small organoid size of PTHS organoids. N=3 independent replicates (dots), 12-30 measured organoids per experiment in each group. (D) Confirmation of Wnt signaling activity increase after treatment of NPCs with agonist CHIR99021, as measured by TOP-Flash functional reporter assay. N=4 subjects per group (symbols), 3 independent replicates per subject. Mean activity (arbitrary units) was normalized to 1 in ‘parents+DMSO’ group. (E) Fluorescence microscopy images of NPCs from parent and PTHS subjects treated with CHIR99021 (or DMSO as a control) after staining for mesoderm marker Brachyury and senescence marker p16^{INK4a} (top row), endoderm marker SOX17 or TCF4 (middle row), and neural lineage marker Nestin and SOX2 (bottom row). Arrowheads in high magnification insets represent co-localization. (F) Treatment of PTHS NPCs with Wnt pathway agonists CHIR99021 (light blue bar) rescues organoid size. N=3 independent replicates (dots), 15-20 measured organoids per experiment in each group. (G and H) Treatment of sPOs (G) and CtOs (H) with Wnt agonist CHIR99021 increases population of NPCs, as revealed by single cell RNA-Seq. Left: comparison of UMAP representation of cellular diversity, showing 6 subpopulations of cells, according to FIG. 6A. Pr-Glut, neural progenitor cells in glutamatergic lineage; IP-Glut, intermediate progenitors in glutamatergic ineage; N-Glut, glutamatergic neurons; Pr-GABA, neural progenitors in inhibitory lineage; IP-GABA, intermediate progenitors in inhibitory lineage; N-GABA, GABAergic interneurons). Other cell types are not shown. Right: quantification of percentages of neural progenitors and neurons. (I) Relative expression of GAD1 and GAD2 in CtO organoids treated with CHIR99021. N=3 biological replicates per condition for parent/child pair #4. (J) Top: UMAP showing expression of TCF4 in PTHS sPOs treated with CHIR99021 (right) in comparison with untreated PTHS sPOs (left). Intensity of purple indicates relative expression level. Bottom: Violin plot showing expression level of TCF4 in single cells (dots) of untreated and CHIR-treated PTHS sPOs. (K) Treatment of PTHS NPCs with CHIR99021 increases expression of TCF4 and TCF4 downstream target gene GADD45G. N=4 subjects per group (symbols), 3 independent replicates per subject. Mean expression level was normalized to 1 in ‘parents+DMSO’ group for each gene. (L) Immunostaining of 4 weeks-old CtOs for β-catenin, showing localization at the centers of rosettes in control organoids, but disorganized staining in PTHS CtOs (arrowhead). (M) Expression levels for CTNNB1 (β-catenin) in NPCs (top left) or progenitors of the excitatory lineage in CtOs (top right). (N) Expression of genes coding for cadherin 23 (CDH23) and protocadherin 15 (PCDH15), which are DE genes between parent and PTHS NPCs (left). Right graphs represent expression levels in NPCs treated with Wnt agonist CHIR99021. Bar graphs represent mean+SEM. n.s., not statistically significantly different, *p<0.05, **p<0.01, ***p<0.001; Welch's t-test in M and N (left graphs) and ANOVA followed by HSD post-hoc test elsewhere. In B, I, K and N (right graphs), mean gene expression was normalized to 1 in each control group. In D, Wnt signaling activity in parents+DMSO groups was set to 1. In K, mean expression in parents+DMSO groups was set to 1 for each gene. In K, statistical comparison is between means of PTHS+CHIR and PTHS+DMSO (control) groups.

FIG. 17A-L shows intermediate progenitors are less abundant in PTHS organoids. (A) Violin plots showing expression of SOX1, SOX3, SOX4, and SOX11 in cellular subpopulations in CtOs and sPOs. See FIG. 13B for SOX2 expression. Pr-Glut, neural progenitor cells in glutamatergic lineage; IP-Glut, intermediate progenitors in glutamatergic ineage; N-Glut, glutamatergic neurons; Pr-GABA, neural progenitors in inhibitory lineage; IP-GABA, intermediate progenitors in inhibitory lineage; N-GABA, GABAergic interneurons. (B and C) Expression of SOX1 (B) and SOX3 (C) in progenitors (Pr-Glut) and intermediate progenitors (IP-Glut) in CtOs, and in progenitors (Pr-GABA) and intermediate progenitors (IP-GABA) in sPOs. Each dot represents a single cell; violin plots represent distribution of gene expression in each population; N=959 and 1230 Pr-Glut cells in parent and PTHS CtO groups, respectively; N=717 and 382 IP-Glut cells in parent and PTHS CtO groups, respectively; N=346 and 1376 Pr-GABA cells in parent and PTHS sPO groups, respectively; N=2737 and 105 IP-GABA cells in parent and PTHS sPO groups, respectively. (D) Relative expression of SOX3 (RT-qPCR) after shRNA-mediated SOX3 knockdown in parental control NPCs in 2D culture. N=3 independent replicates (dots), 2 technical replicates per sample. There is a tendency for lower expression, which was short of significant (P≈0.50) but in the expected direction. Mean expression in the parent+control shRNA group was normalized to 1. NPCs used were from parent #4 line (Table 1). (E) Relative expression of CDKN2A, ASCL1, and HES1 (RT-qPCR) after shRNA-mediated SOX3 knockdown in parental control NPCs in 2D culture. N=3 independent replicates (dots), 2 technical replicates per sample. NPCs used were from parent #4 line (Table 1). (F) Live cell count in parent and PTHS NPCs subjected to SOX3 over-expression. N=3 biological replicates, with cells from parent/child pair #4. (G) Relative expression of SOX3 after SOX3 over-expression. N=3 biological replicates, with cells from parent/child pair #4. (H) SOX4 expression is normal in intermediate progenitors (IP-GABA) and neurons (N-GABA) in PTHS sPOs. Each dot represents a single cell; violin plots represent distribution of gene expression in each population; N=2737 and 105 IP-GABA cells in parent and PTHS groups, respectively; N=2661 and 988 N-GABA neurons in parent and PTHS groups, respectively. Although expression was not changed per cell, as seen for CtOs (FIG. 10G), there is a clear reduction in the number of both intermediate progenitors and neurons in the PTHS organoids. Color code for violin plots is the same as in B. (I) SOX11 expression is normal in intermediate progenitors (IP-Glut and IP-GABA) and neurons (N-Glut and N-GABA) in PTHS CtOs and sPOs. Each dot represents a single cell; violin plots represent distribution of gene expression in each population; N=717 and 382 IP-Glut cells in parent and PTHS CtO groups, respectively; N=1401 and 380 N-Glut neurons in parent and PTHS CtO groups, respectively; N=2737 and 105 IP-GABA cells in parent and PTHS sPO groups, respectively; N=2661 and 988 N-GABA neurons in parent and PTHS sPO groups, respectively; N=717 and 382 IP-Glut cells in parent and PTHS CtO groups, respectively. Color code for violin plots is the same as in B. (J) Quantification of percentages of MAP2+ cells in 2D cultures of differentiating neurons derived from parental controls and PTHS subjects. N=4 subjects per group (symbols), 2 independent differentiation expts., 3 independent replicates per subject, 4 counted randomly chosen fields of view per independent replicate. (K) UMAP representation of single cell RNA-Seq results in PTHS and parental control CtOs and sPOs, highlighting the intermediate progenitors in red in each plot. The percentages of IPs are displayed at the left bottom for each quadrant. (L) Left: Violin plots showing expression of POU3F2 (which encodes BRN2, expressed in intermediate progenitors) in IPs and neurons of CtOs and sPOs. Each dot represents a single cell. Right: Severe reduction in the percentage of intermediate progenitors in PTHS CtOs and sPOs, as judged from quantification of cellular populations in single cell RNA-Seq data. Color code for violin plots is the same as in B. Bar graphs represent mean+SEM. n.s., not statistically significantly different, *p<0.05, **p<0.01, ***p<0.001; Welch's t test assuming unequal variances (E and J), Kruskal-Wallis H test to compare gene expression in PTHS versus respective parent (B, C, H, I, and left panel in L). In D, comparisons between parent and respective PTHS groups yielded statistical significance at the limit p-value of 0.5. t indicates that the PTHS means is statistically significantly different from the parent means but the fold change is less than 10%.

FIG. 18A-O provide details on genetic correction of TCF4 expression. (A) Normalized transcriptional activity from alternative promoters in the TCF4 locus (red bars), in parent and PTHS samples (first row). The second row depicts a schematic representation of the TCF4 locus, showing the location of its exons. The position of the designed gRNAs (blue arrows) is shown for the three chosen TCF4 alternative promoters, upstream of exons 3b, 8a and 10a. The remaining rows show the transcripts formed from transcription initiated at exons 3b, 8a and 10a, which yield TCF4 protein isoforms TCF4-B, TCF4-D, and TCF4-A, respectively. (B) Testing of transactivation efficiency of five TCF4 gRNAs on the expression of TCF4 in SH-S5Y5 cells. scr gRNA, control scrambled gRNA; no gRNA, empty expression construct. N=4 independent replicates, 3 technical replicates. (C) Left: CNTNAP2 relative expression in 2D neuronal cultures. N=3 (control) or 4 (PTHS) subjects (symbols), 3 independent replicates per subject, 2 technical replicates. Bottom: Trans-epigenetic TCF4 expression correction increases CNTNAP2 in SH-S5Y5 cells. N=4 independent replicates, 3 technical replicates. Right: Relative expression (RT-qPCR) of TCF4 target gene KCNQ1 is greatly increased in PTHS neurons. N=4 subjects per group (symbols), 3 independent replicates per subject, 2 technical replicates per sample. Bottom: TCF4 expression correction decreases KCNQ1 expression in transfected SH-S5Y5 cells. (D) Top, Increase in TCF4 expression levels after TCF4 correction. Bottom, ratio between expression abundance for the normal (C at position 959 of the coding sequence) and mutated (T at that position) TCF4 alleles. N=3 independent replicates per group (dots), 10 pooled organoids per sample. (E) Expression levels GADD45G (TCF4 downstream target gene), CDKN2A, SOX3 and MAP2 after TCF4 correction. N=3 independent replicates per group (dots), 10 pooled organoids per sample. (F) Correction of DCX expression levels after TCF4 correction. N=3 independent replicates per group (dots), 10 pooled organoids per sample. (G) Relative expression of DCX in post-mortem PTHS cortex tissue. (H) DCX expression is reduced in intermediate progenitors (IP-Glut and IP-GABA) and neurons (N-Glut and N-GABA) in PTHS CtOs and sPOs. N=717 (parent) and 382 (PTHS) IP-Glut cells, 1401 (parent) and 380 (PTHS)N-Glut neurons, 2737 (parent) and 105 (PTHS) IP-GABA cells, or 2661 (parent) and 988 (PTHS)N-GABA neurons. (I) Significantly lower expression abundance (in transcripts per million, or TPM) of DCX in PTHS neurons in comparison with neurons derived from parental control, as judged from RNA-Seq data. N=3 independent replicates per group (dots). (J) Validation of correction of TCF4 expression with overexpression cassettes (top) containing the cDNA coding for TCF4-B isoform preceded by a synthetic promoter containing micro-E5 (pE5) regulatory binding sites, which allows overexpression in TCF4-expressing cells, thereby preventing ectopic expression. Versions of this construct (with 6 or 12 μE5 boxes) were transfected into PTHS NPCs in 2D culture, followed by RT-qPCR evaluation of increase in expression of TCF4 and TCF4 target gene GADD45G. N=3 independent replicates per group. Means of relative expression levels are compared for cells transfected with overexpression cassettes containing 6 or 12 μE5 boxes against control with a minimal promoter (minP; not expected to increase TCF4 levels). Expression of each gene in the parent group is normalized to 1. (K) Top row, Relative expression of TCF4 and CDKN2A in CtOs subjected to TCF4 OE with lentiviral vectors applied at the beginning of the organoid derivation protocol. Bottom row, Densities of SOX2 and CTIP2-positive cells in CtOs subjected to TCF4 OE. N=3 biological replicates per subject, for organoids from parent/child pairs #1 and #4. (L) Representative raster plots showing differences in firing activity between parent (left) and PTHS (top right) CtOs in MEA assay, but partial rescue of activity in PTHS organoids treated with TCF4 OE (bottom right) lentiviral vectors. (M) Low magnification images of 8 weeks-old CtOs subjected to transduction with OE AAV vector containing 12 μE5 boxes (TCF4 OE). (N) Top row, Relative expression of TCF4 and CDKN2A in CtOs subjected to TCF4 OE with AAV vectors applied after the end of the neural induction phase. Bottom row, Densities of SOX2 and CTIP2-positive cells in CtOs subjected to TCF4 OE. N=3 biological replicates per subject, for organoids from parent/child pairs #1 and #4. (O) Current mechanistic model to explain aberrant cellular phenotypes in PTHS neural structures. Due to TCF4 haploinsufficiency in PTHS, Wnt signaling activity diminishes, in turn leading to decreased SOX3 expression in NPCs, impairing proliferation. Moreover, SOX4 was also downregulated in PTHS cells, which suggests it impairs neuronal differentiation and content in PTHS neural tissue. scr gRNA, scrambled (control) guide RNA. Bar graphs represent mean+SEM. *p<0.05, **p<0.01, ***p<0.001; Welch's t test (D) or ANOVA followed by Tukey-Kramer's HSD post-hoc test (C).

### DETAILED DESCRIPTION

As used herein and in the appended claims, the singular forms “a,” “an,” and “the” include plural referents unless the context clearly dictates otherwise. Thus, for example, reference to “a promoter” includes a plurality of such promoters and reference to “the construct” includes reference to one or more constructs, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that any publication is prior art. Moreover, with respect to any term that is presented in one or more publications that is similar to, or identical with, a term that has been expressly defined in this disclosure, the definition of the term as expressly provided in this disclosure will control in all respects.

Transcription Factor 4 (TCF4; OMIM 602272) encodes a helix-loop-helix transcription factor implicated in several aspects of neural development, including neurogenesis, cell survival, cell cycle regulation, neuronal differentiation, neural lineage commitment, and neuronal excitability. Numerous alternative transcripts are transcribed from the TCF4 locus, some of which are highly expressed during brain development.

Many studies found TCF4 gene variants to be genetically associated with a range of neuropsychiatric diseases, namely, schizophrenia, bipolar disorder, post-traumatic stress disorder, and major depression disorder. Importantly, de novo heterozygous mutations in TCF4 cause an autism spectrum disorder known as Pitt-Hopkins Syndrome (MIM #610954), and similar syndromes have been shown to be caused by mutations in NRXN1β and CNTNAP2, which are TCF4 downstream target genes. Despite this knowledge, little is known about the molecular and cellular mechanisms through which mutations in TCF4 lead to alterations in neural development and function.

PTHS includes severely debilitating clinical symptoms, such as profound cognitive impairment, developmental delay, generalized hypotonia, breathing abnormalities, seizures, lack of speech, typical autistic behaviors, chronic constipation, and a distinctive facial gestalt. Most PTHS patients display private TCF4 mutations, which may be large chromosomal deletions spanning the whole gene, partial gene deletions, translocations, frameshift, nonsense, splice site or missense mutations, most of which are regarded as loss-of-function mutations that impair TCF4's transcriptional activity.

Several transgenic mouse lines carrying TCF4 mutations have been produced as PTHS animal models. Some of these lines display PTHS-like phenotypes, including deficits in social interaction, associative memory, sensorimotor gating, and altered gastrointestinal transit. Examination of brain tissue from these animals revealed abnormal cortical development, altered neuronal migration during hippocampal and pontine nuclei development, and impaired oligodendrocyte differentiation. However, these mice do not display the full array of clinically relevant symptoms, including many of the most debilitating ones such as severe motor delay and hypotonia. Moreover, only a few of these mouse lines carry heterozygous mutations like those found in PTHS patients.

In order to study TCF mutations, the disclosure used neural progenitor cells (NPCs) and neurons differentiated in vitro from patient-derived induced pluripotent stem cells (iPSCs), allowing for analysis of the disease's influence on individual cell types under relevant genomic context. In addition, patterned cortical organoids—including pallium and subpallium-type organoids containing excitatory and inhibitory neuronal lineages were also generated. These three-dimensional (3D) neural structures consistently display a range of different cell populations and have been successfully used to model cellular pathology during early neurodevelopment in several disorders.

The disclosure demonstrates that PTHS cortical organoids are aberrant in size and structure, containing a higher percentage of NPCs and fewer neurons than control organoids. PTHS-derived NPCs exhibit reduced proliferation and impaired ability to differentiate into neurons. Significantly, molecular probing of these neural systems unraveled a pathological mechanism through which mutations in TCF4 lead to reduced canonical Wnt/β-catenin signaling, which then leads to reduced expression of SOX transcription factors, resulting in cellular abnormalities. The disclosure also demonstrates the pharmacological manipulation of Wnt signaling and that genetically corrected expression of TCF4 itself, results in restoration of neural characteristics at the cellular level. Taken together, the data of the disclosure reveal novel cellular and molecular PTHS phenotypes in relevant human cell types and show that these are reversible, providing routes for therapeutic intervention in patients with PTHS or other genetic diseases associated with TCF4.

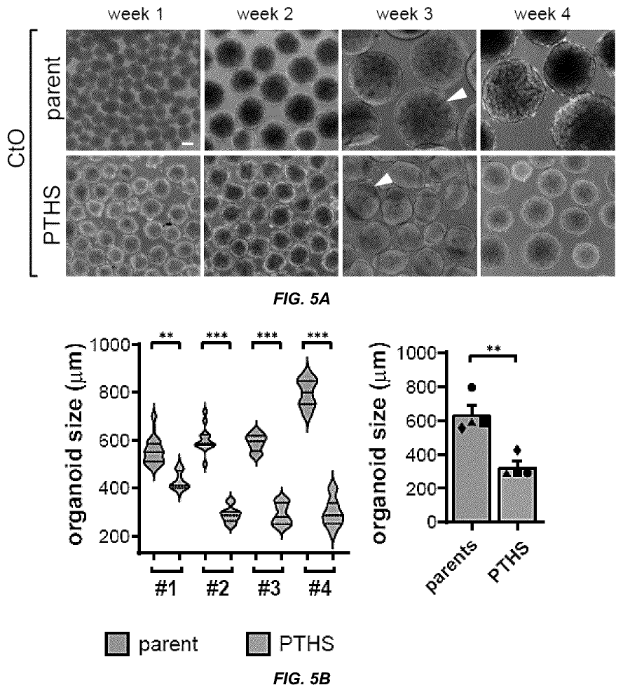

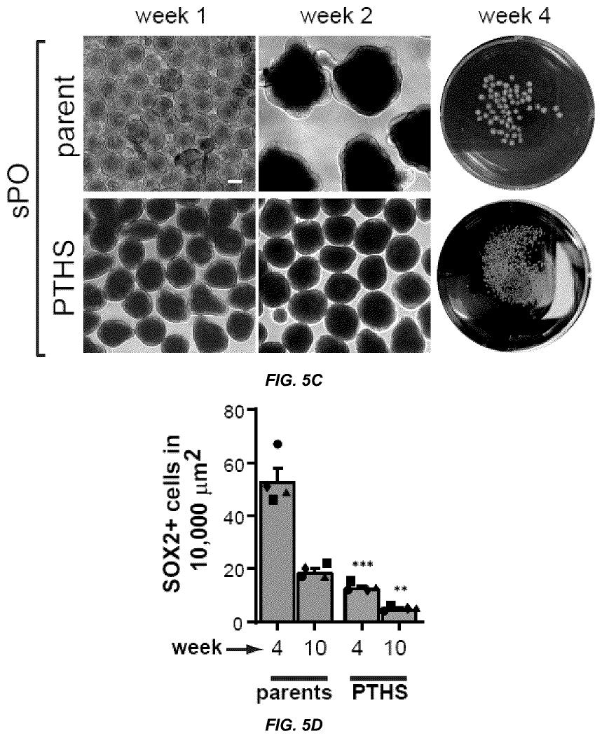

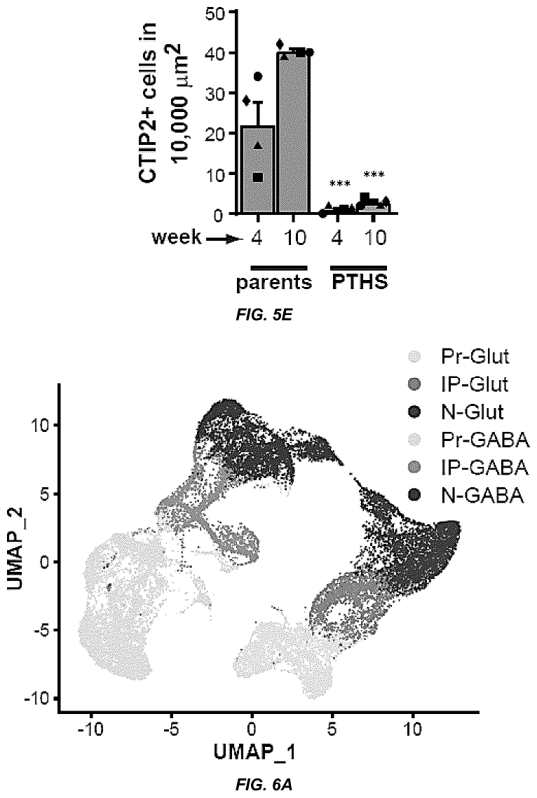

The patient-derived brain organoids of two types (pallial and subpallial) in combination with neural 2D culture systems were used to investigate the pathophysiology and aberrant molecular mechanisms associated with clinically relevant mutations in TCF4. These cells were derived from pediatric patients suffering from PTHS, a devastating autism spectrum condition solely caused by TCF4 mutations. The disclosure demonstrates that PTHS NPCs proliferate at a slower rate and display impaired neuronal differentiation. Moreover, that PTHS organoids exhibit abnormal electrical properties and contain fewer cortical neurons (FIGS. 5-7).

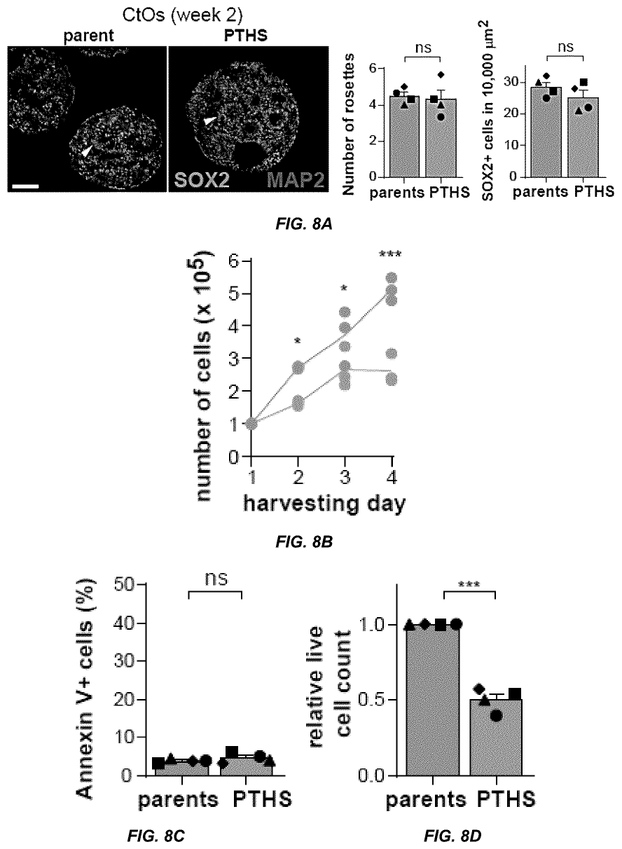

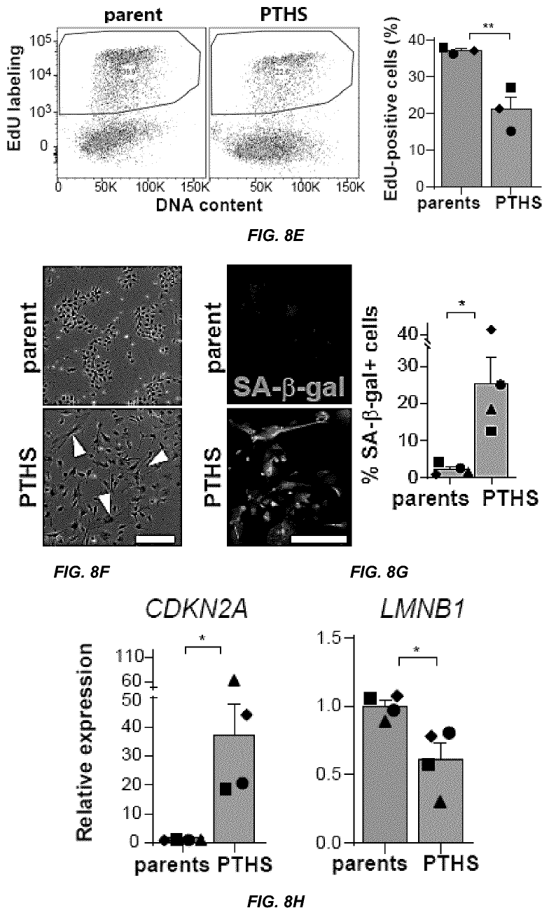

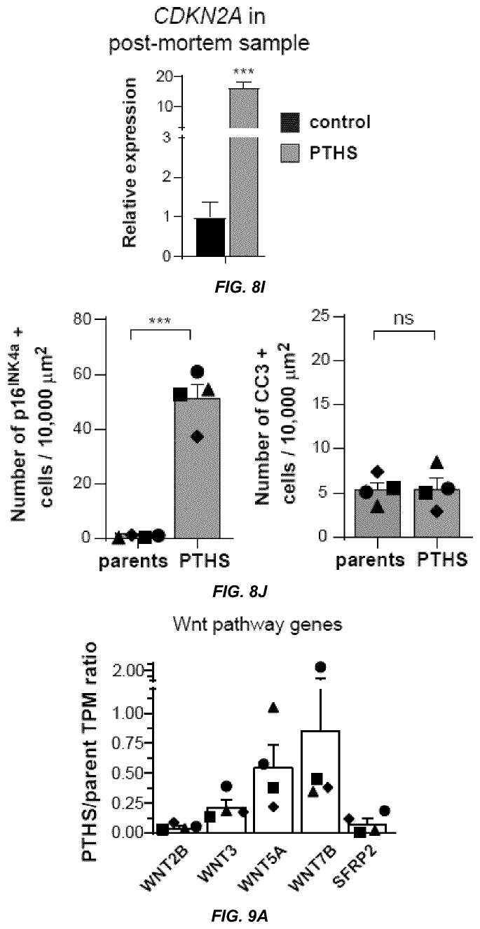

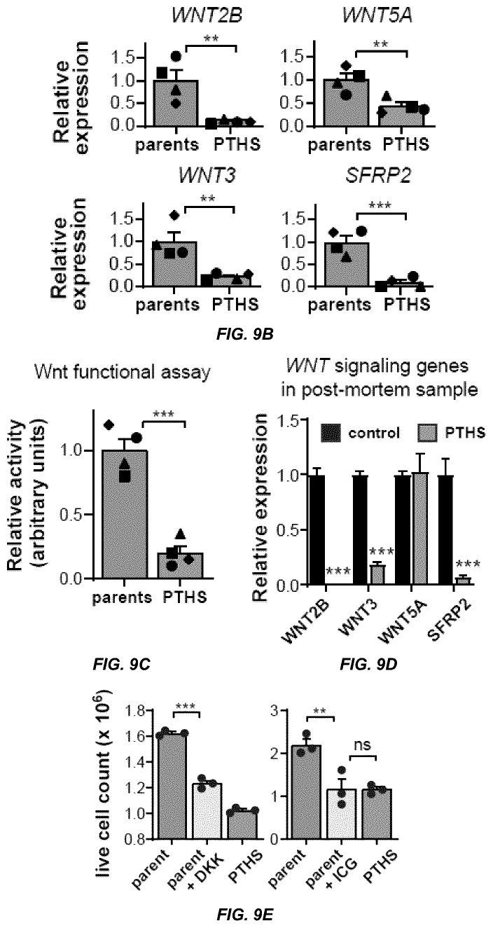

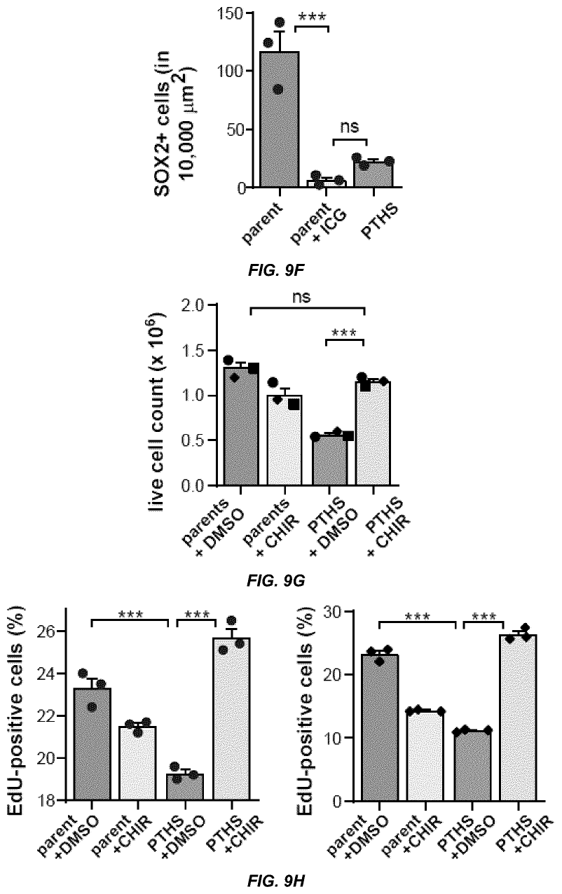

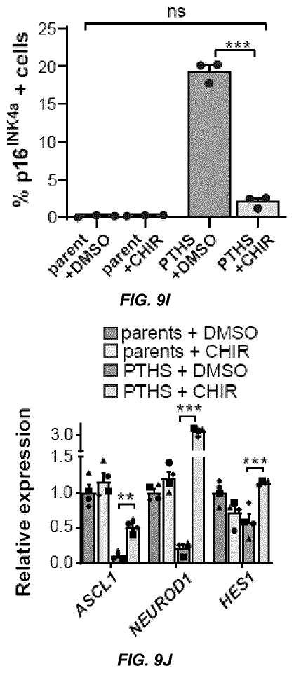

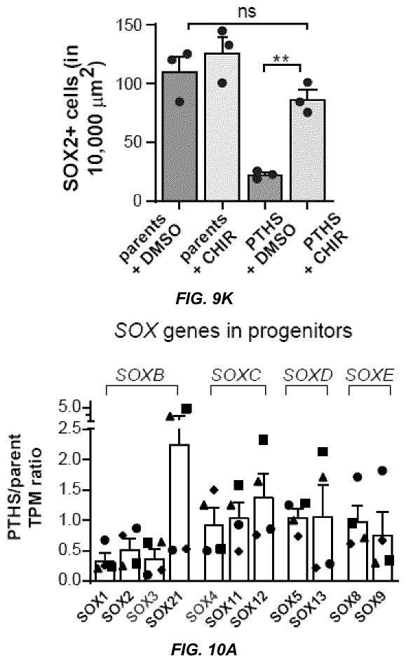

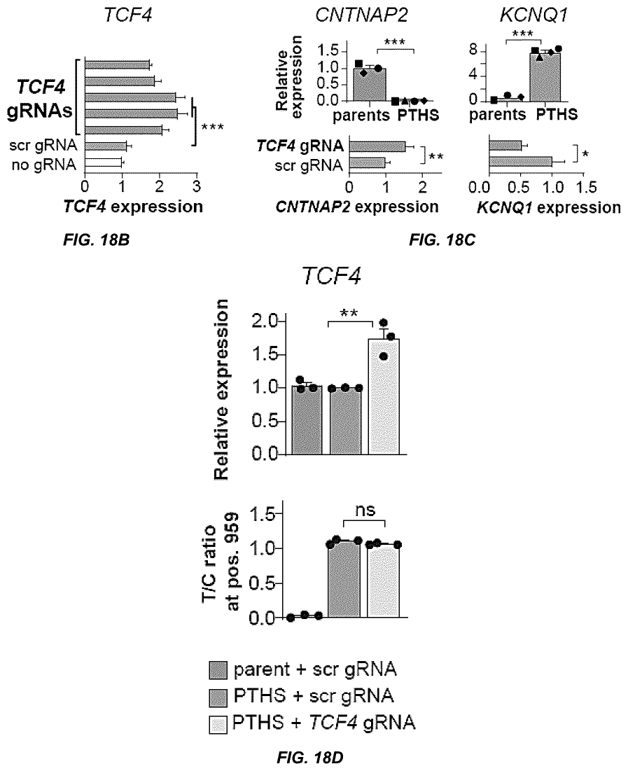

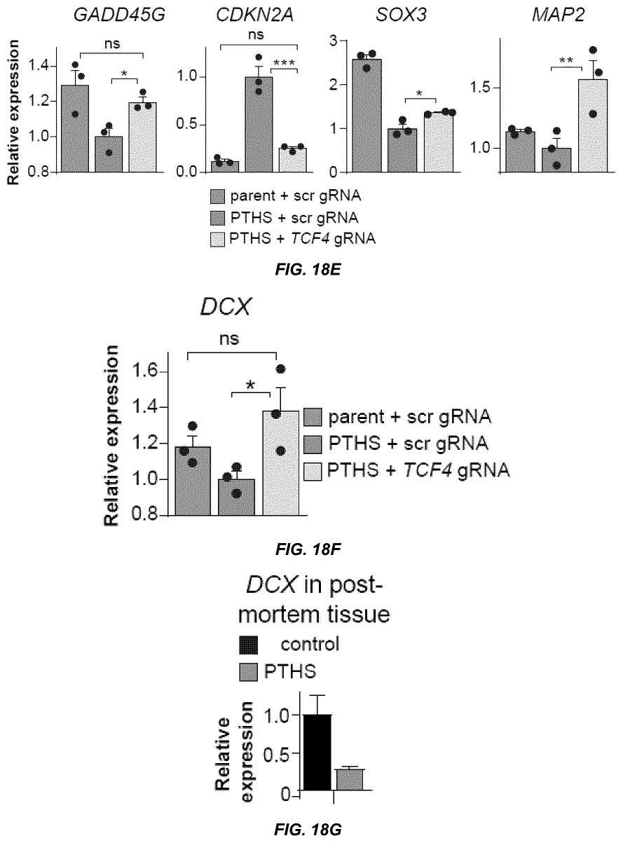

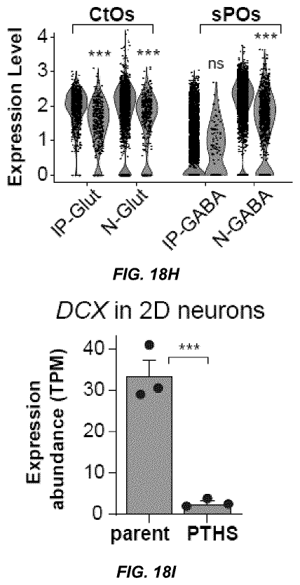

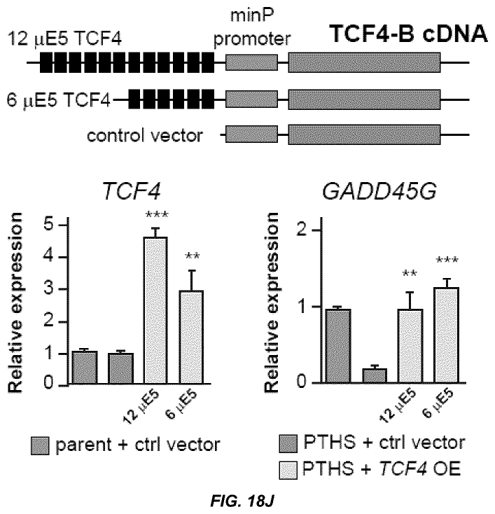

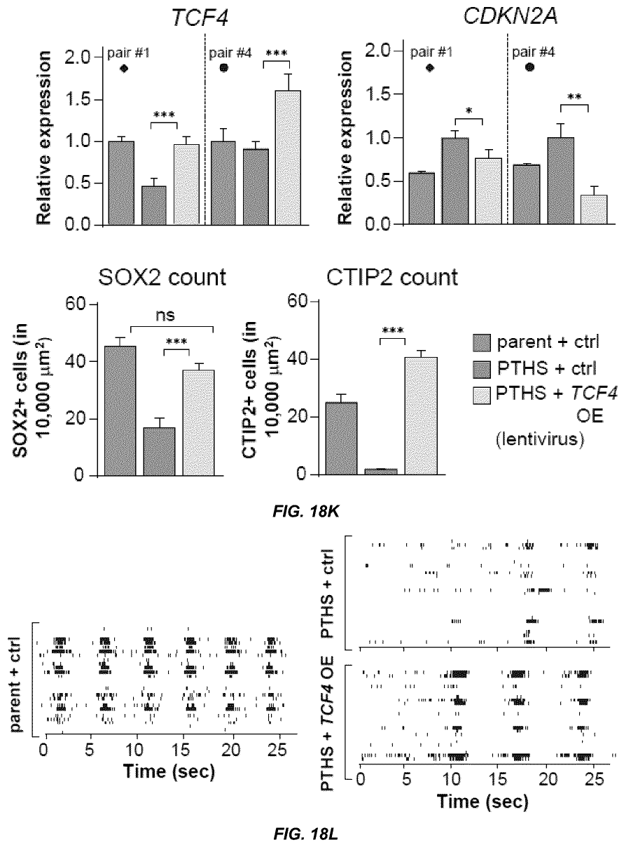

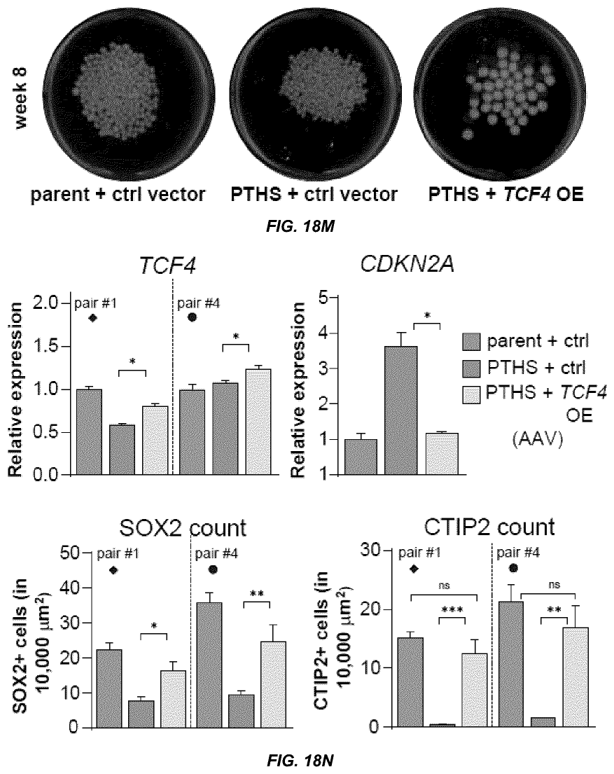

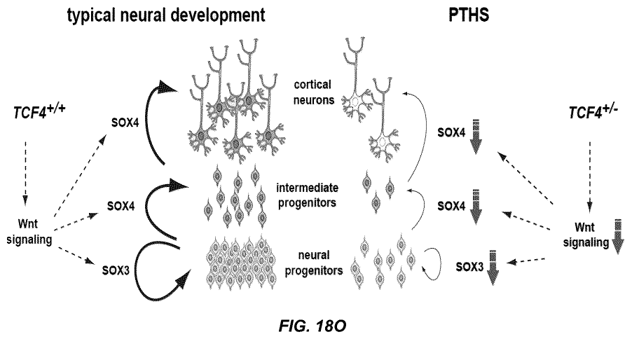

The disclosure demonstrate a model (FIG. 18O) according to which the pathological molecular mechanism includes a chain of molecular events that leads from TCF4 loss-of-function mutations to decreased Wnt signaling activity in the lowly proliferative PTHS NPCs (FIG. 8). The data show that Wnt signaling is mechanistically downstream of TCF4, in a clear cascade that is dysregulated in patient cells, a result that provides for therapeutic interventions and a better understanding of disease pathology. Pharmacological activation of Wnt signaling in PTHS samples can completely correct the aberrant NPC proliferation phenotype, the morphology of organoids, and the expression of senescence markers and of downstream molecular players (FIG. 9), providing for pharmacological therapy.

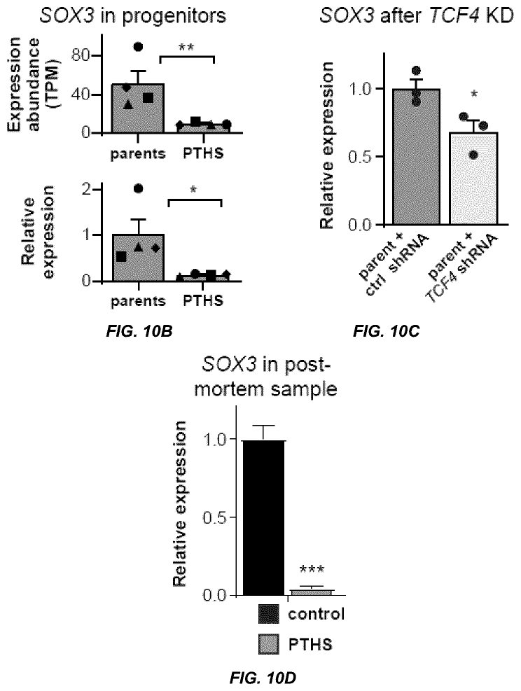

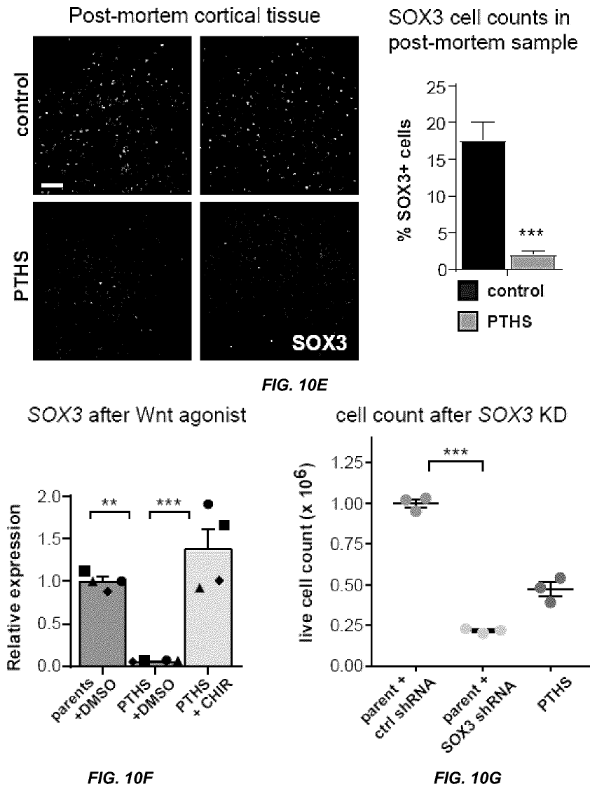

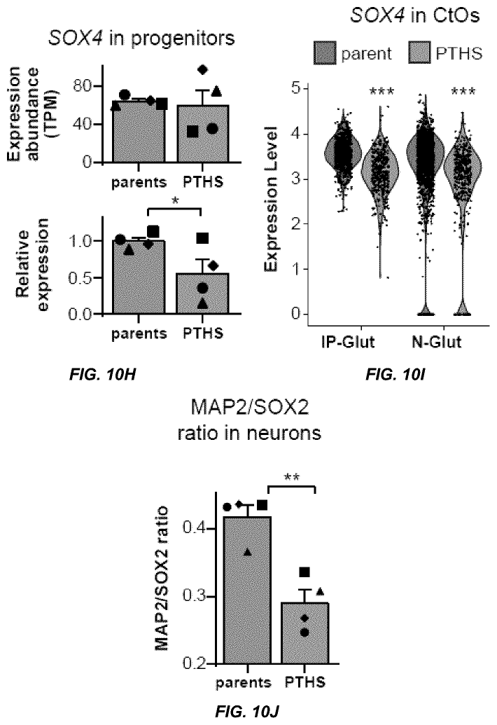

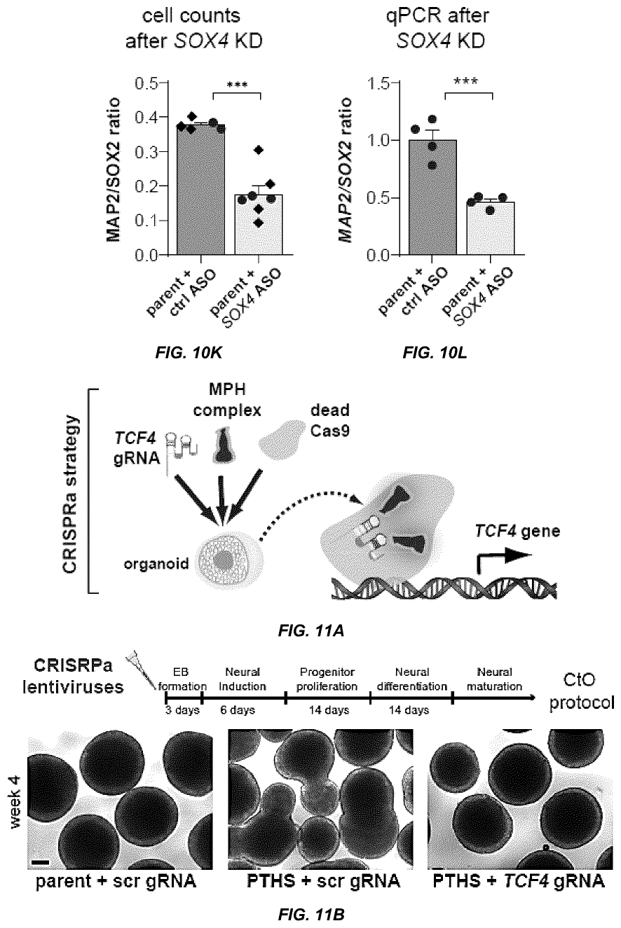

The disclosure also provides mechanistic evidence that Wnt signaling controls the expression of two SOX transcription factors, SOX3 and SOX4 (FIG. 10). A diminishment in the expression of SOX3 in PTHS NPCs, in organoids and in the post-mortem cortical sample, and SOX3 downregulation was found to cause reduced NPC proliferation (FIG. 10). Interestingly, mutations in SOX3 have been associated with another neurodevelopmental disorder, X-linked mental retardation, suggesting the existence of an overlapping molecular mechanism between such a condition and PTHS.

PTHS NPCs also exhibit impaired differentiation into neurons, in keeping with the pro-neural roles of helix-loop-helix transcription factors NEUROG1, NEUROG2 and ASCL1, which are known to interact with TCF4. Interestingly, SOX4 expression was found to be diminished in PTHS NPCs and in intermediate progenitors and neurons of PTHS organoids (FIG. 10). SOX4 transcription factor is known to participate in neuronal differentiation, which is consistent with the findings that PTHS organoids contain fewer neurons, and that patient-derived NPCs have slower differentiation rates than control cells, a phenotype also observed when the expression of SOX4 was knocked down in differentiating neuronal cultures (FIG. 10). It is hypothesized that TCF4 haploinsufficiency leads to SOX4 downregulation, resulting in decreased neuronal differentiation (FIG. 18O).

The deficits in cell proliferation and differentiation are factors contributing to the lower content of cortical neurons that are observed in both PTHS organoids and in the post-mortem brain tissue from a PTHS individual. It should be noted that the decreased neuronal content in the organoids and post-mortem sample are consistent with the detection via MRI of small or absent corpus callosum in some PTHS children. It is noteworthy that the PTHS neural tissue exhibits such level of disorganization and reduction in the content of cortical neurons, and it will be interesting to determine which clinical symptoms arise from these abnormalities or whether this effect manifests during neural development or in the fully formed nervous system.

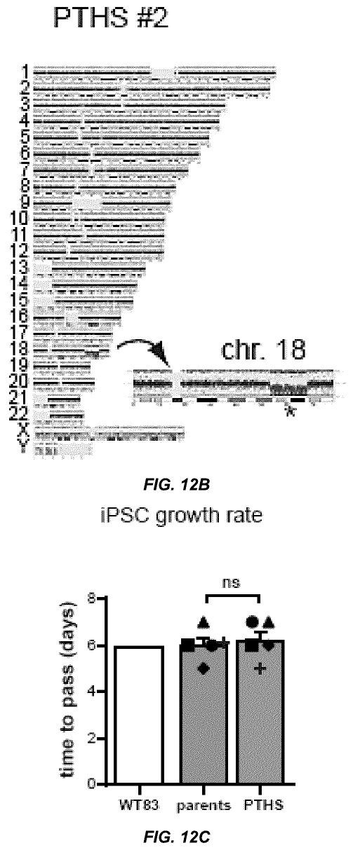

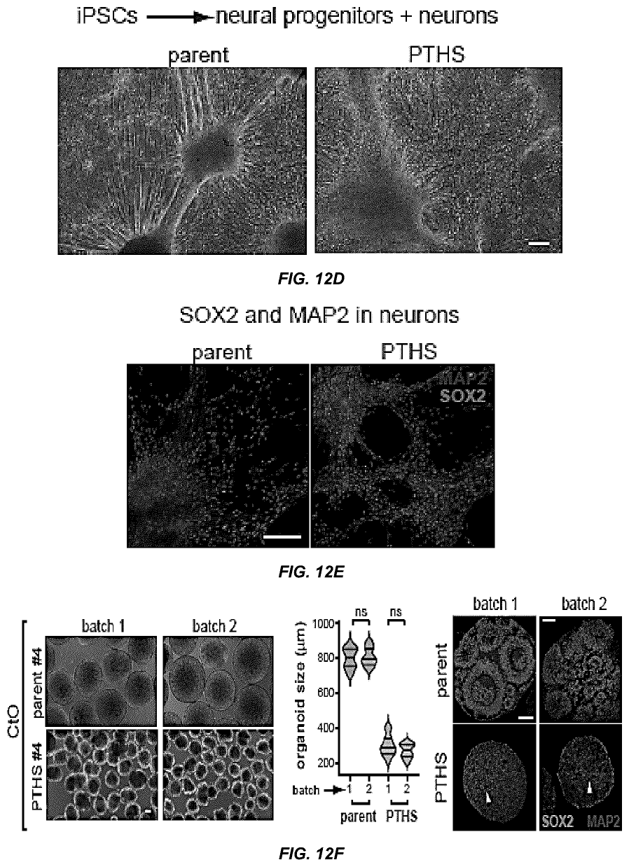

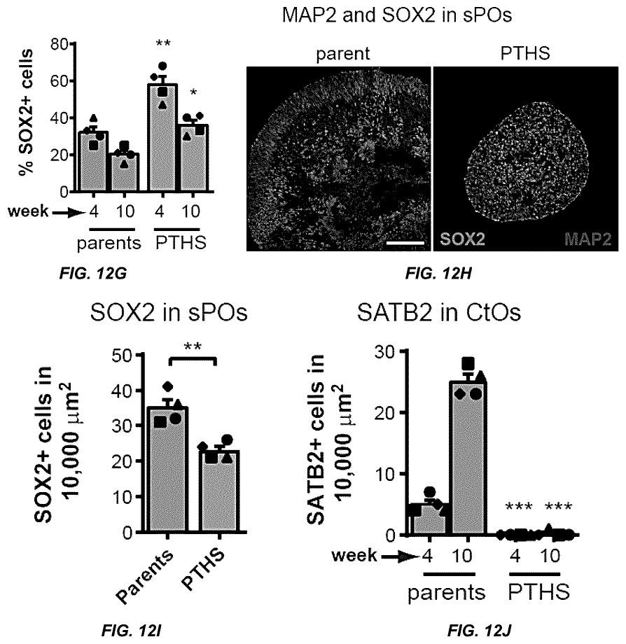

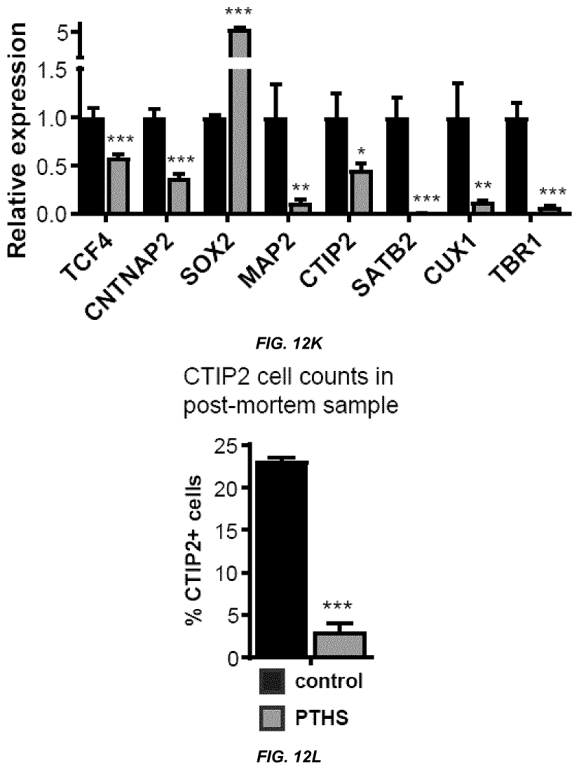

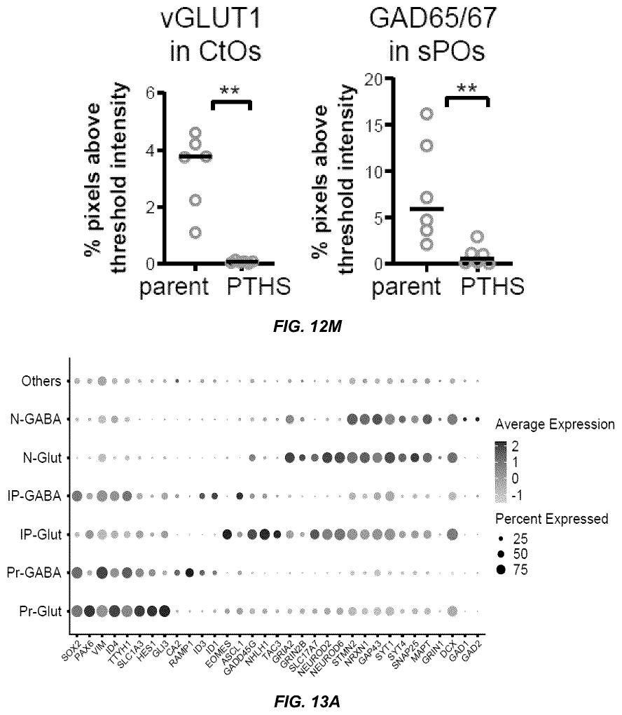

Tcf4 full knockout mice carrying homozygous loss-of-function mutations exhibit substantially altered populations of cortical neurons, including SATB2- and BRN2-expressing cells, but these alterations are significantly milder in Tcf4^{+/-} mice, which exhibit phenotypes certainly less prominent than the levels of tissue disorganization and altered gene expression in the post-mortem PTHS cortical sample (FIGS. 5 and 12). This suggests that mouse models are not ideal for studying TCF4 heterozygous mutations, like those seen in PTHS patients. In contrast, the data provided herein show severe impairment of cortical neuron differentiation in PTHS organoids, in keeping with the observations in post-mortem brain tissue, signifying that brain organoid models provide a novel window of opportunity to observe neurodevelopmental abnormalities relevant to PTHS. The difference in phenotypic severities between the brains of Tcf4^{+/-} mice and PTHS human organoids or post-mortem sample may reflect important evolutionary distinctions between human and rodent neurodevelopment and, thus, justify the use of patient-derived systems to better understand pathophysiology in the context of this and other neurodevelopmental conditions.

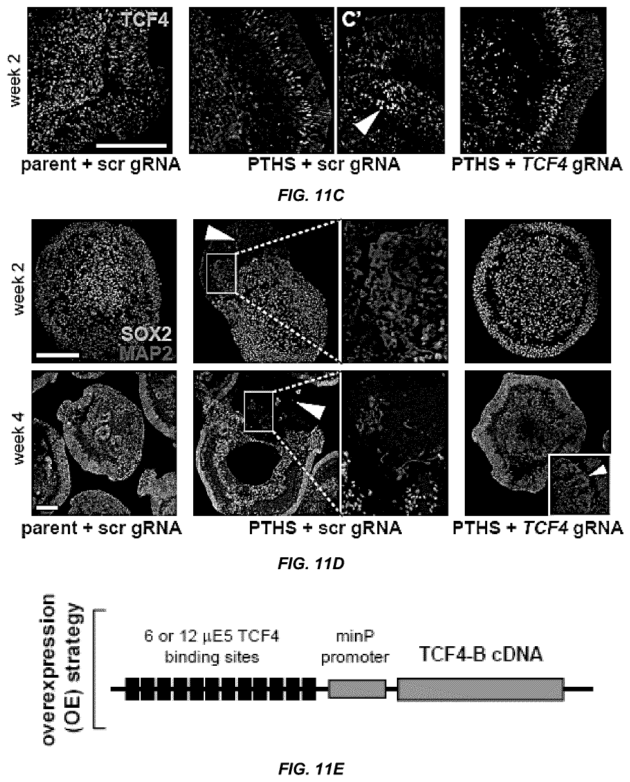

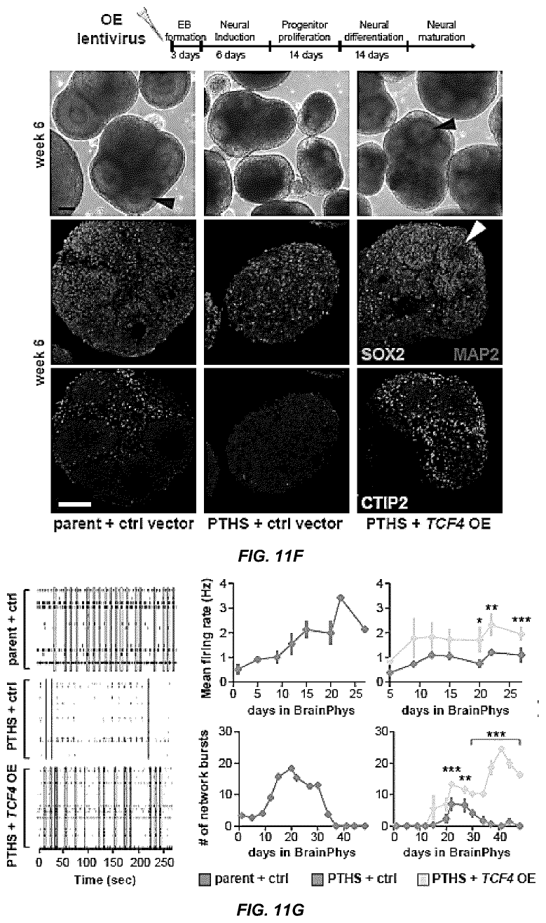

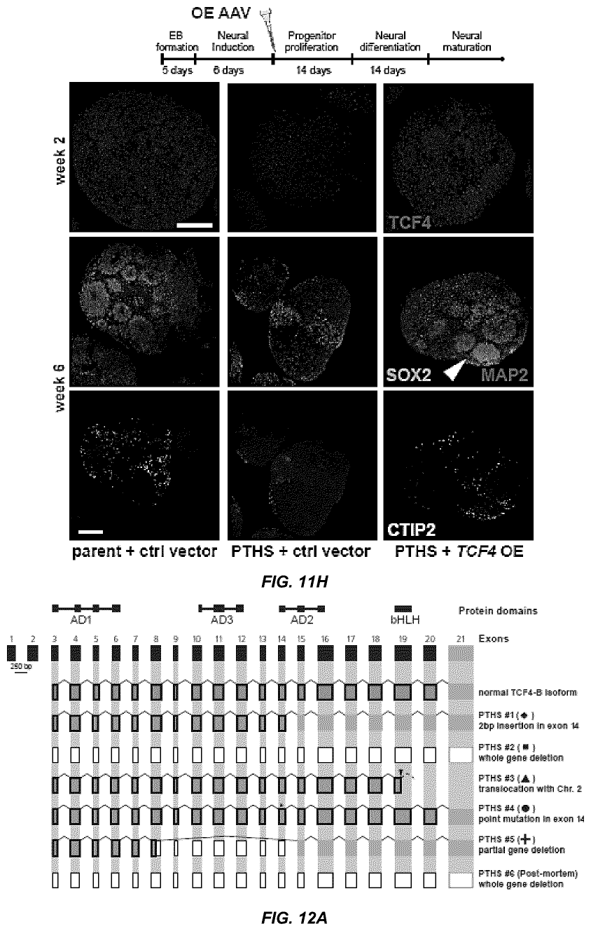

The disclosure also demonstrates a series of genetic manipulative experiments to enhance the expression of TCF4 and therefore correct its expression in PTHS neural tissue in vitro (FIG. 11). These approaches, which included over-expression of an extra TCF4 gene copy and CRISPR-mediated trans-epigenetic enhancement of expression from the endogenous TCF4 locus, resulted in the reversal of aberrant cellular phenotypes, an important finding that may direct therapeutic efforts to treat PTHS. Furthermore, because the CRISPR-mediated correction of TCF4 expression enhances transcription from both the mutated and normal endogenous alleles, this experiment definitively proves that the PTHS phenotypes observed here are caused by haploinsufficiency and not by a dominant negative effect. The methods and compositions of the disclosure also provide benefit in the comprehension of other genetic diseases, including autosomal recessive intellectual disability conditions classified as Pitt-Hopkins-like syndromes, which are caused by mutations in the TCF4 downstream target genes NRXN10 and CNTNAP2, as well as schizophrenia and other diseases that may have TCF4 as a genetic component.

The practice of the technology described herein will employ, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; Herzenberg et al. eds (1996) Weir's Handbook of Experimental Immunology, 5th edition; and/or more recent editions thereof.

The terminology used in the description herein is for the purpose of describing particular embodiments only and is not intended to be limiting of the disclosure.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

A “protein” or “polypeptide”, which terms are used interchangeably herein, comprises one or more chains of chemical building blocks called amino acids that are linked together by chemical bonds called peptide bonds.

The term “about,” as used herein can mean within an acceptable error range for the particular value as determined by one of ordinary skill in the art, which can depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are disclosed in the prior art. Non-limiting exemplary serotypes useful for the purposes disclosed herein include any of the 11 serotypes, e.g., AAV2 and AAV9. The term “lentivirus” as used herein refers to a member of the class of viruses associated with this name and belonging to the genus lentivirus, family Retroviridae. While some lentiviruses are known to cause diseases, other lentivirus are known to be suitable for gene delivery. See, e.g., Tomas et al. (2013) Biochemistry, Genetics and Molecular Biology: “Gene Therapy—Tools and Potential Applications,” ISBN 978-953-51-1014-9, DOI: 10.5772/52534.

The term “Cas9” can refer to a CRISPR associated endonuclease referred to by this name. Non-limiting exemplary Cas9s include Staphylococcus aureus Cas9, nuclease dead Cas9, and orthologs and biological equivalents each thereof. Orthologs include but are not limited to Streptococcus pyogenes Cas9 (“spCas9”), Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112. For example, UniProtKB G3ECR1 (CAS9_STRTR)) as well as dead Cas9 or dCas9, which lacks endonuclease activity (e.g., with mutations in both the RuvC and HNH domain) can be used. The term “Cas9” may further refer to equivalents of the referenced Cas9 having at least about 60%, 65%, 70%, 75%, 80%, 85%, 90%, 95%, or 99% identity thereto, including but not limited to other large Cas9 proteins. In some embodiments, the Cas9 is derived from Campylobacter jejuni or another Cas9 orthologs 1000 amino acids or less in length.

As used herein, the term “cassette” or “expression cassette” refers to a modular polynucleotide construct that can comprise one or more domains such that the cassette can be effectively transferred between different vector systems and when expressed provides a substantially similar encoded construct or expression profile.

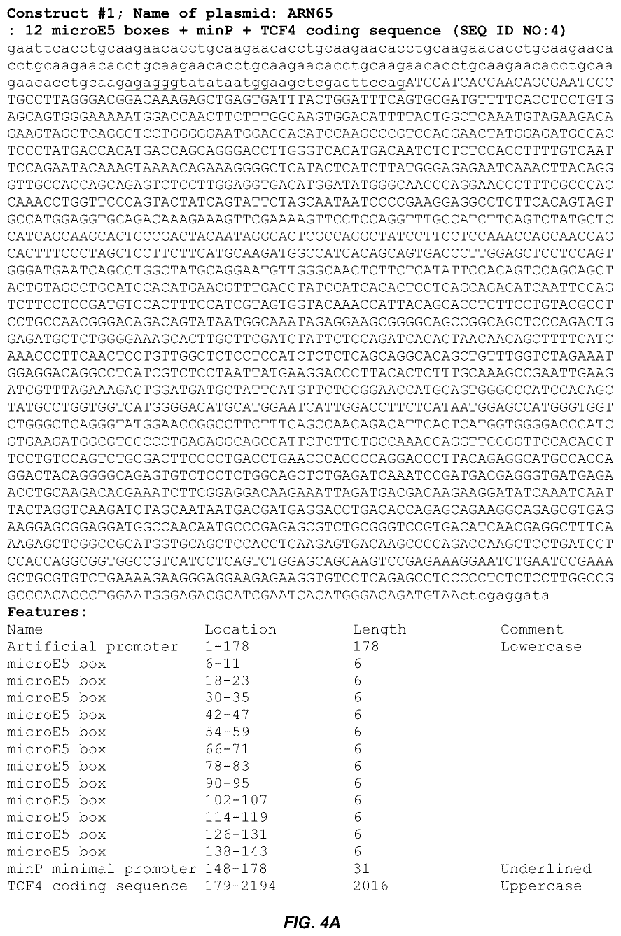

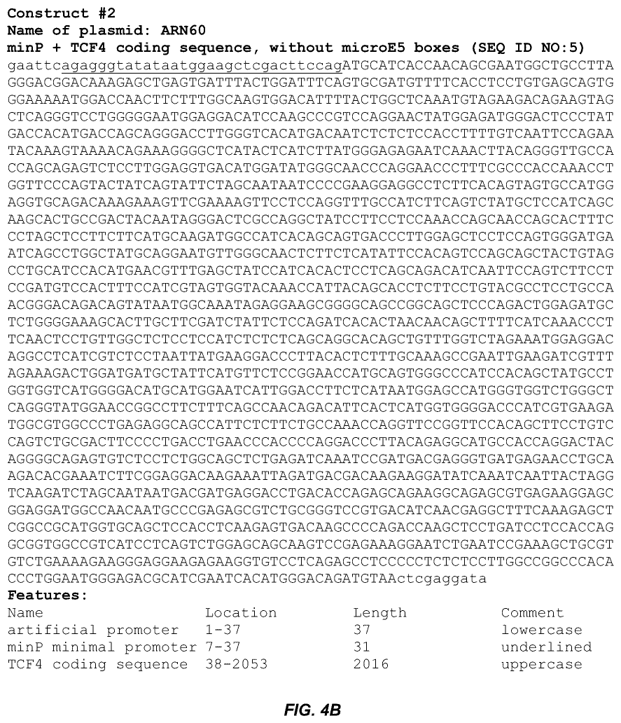

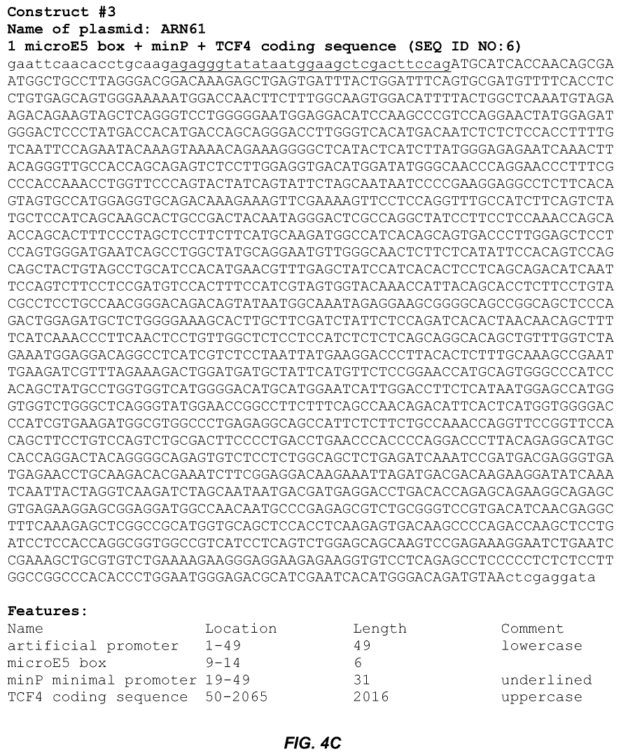

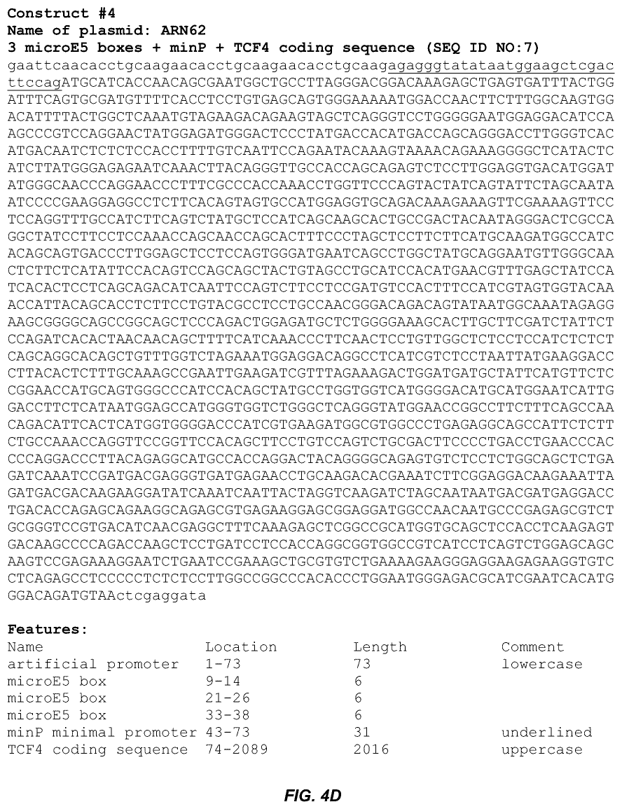

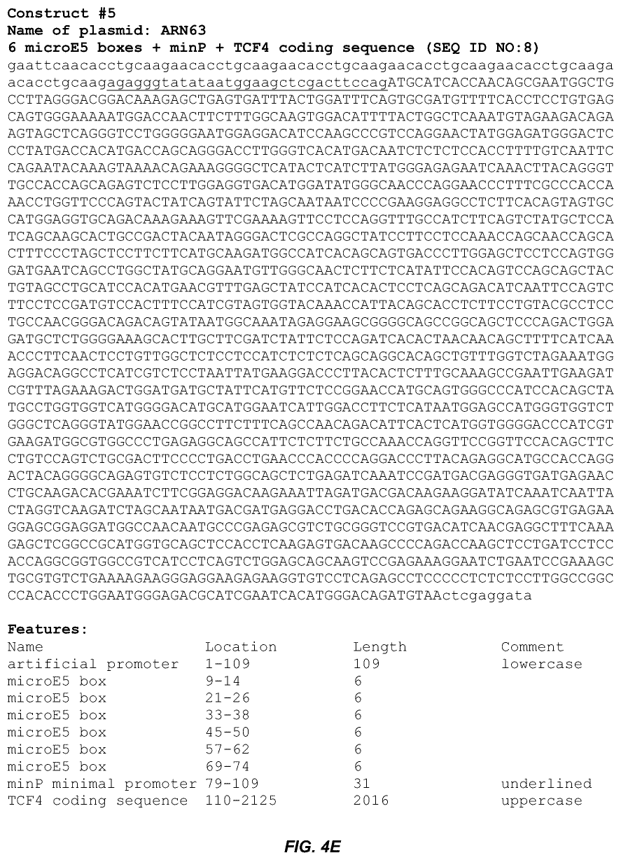

By “TCF4 cassette” is cassette containing a TCF4 coding sequence. In one embodiment, a TCF4 cassette comprises at least one mini-promoter cassette or a core-promoter cassette operably linked to a polynucleotide encoding a TCF4 polypeptide, such as a TCF4-B polypeptide. In some embodiments, the TCF4 cassette can comprise one or more pE5 boxes. Accordingly, a “TCF4 cassette” can comprise a single mini-promoter operably linked to a coding sequence for a TCF4 polypeptide (e.g., a TCF4-B polypeptide), and can comprise one or more pE5 boxes operably associated with a mini-promoter. Examples of cassettes are provided in FIG. 4A-E (SEQ ID NOs: 4, 5, 6, 7, and 8, respectively). It will be recognized that the sequences provided in FIG. 4 can be varied by 1% to 15% (e.g., 85%-99% identical to the sequences in FIG. 4A-D or E so long as the variants can still drive transcription of a functional TCF4 polypeptide.

As used herein, the term “CRISPR” can refer to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” can refer to a type of genetic engineering in which the nucleotide sequence of a target polynucleotide is changed through introduction of deletions, insertions, single stranded or double stranded breaks, or base substitutions to the polynucleotide sequence. In some aspect, CRISPR-mediated gene editing utilizes the pathways of nonhomologous end-joining (NHEJ) or homologous recombination to perform the edits. Gene regulation can refer to increasing or decreasing the production of specific gene products such as protein or RNA.

The term “deficiency” as used herein can refer to lower than normal (physiologically acceptable) levels of a particular agent. In context of a protein, a deficiency can refer to lower than normal levels of the full-length protein.

As used herein, the term “domain” can refer to a particular region of a polypeptide or polynucleotide and is associated with a particular function. For example, “a domain which binds an RNA binding protein” can refer to the domain of a polynucleotide that binds one or more polypeptides that control expression.

The term “encode” as it is applied to polynucleotides can refer to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality.

The term “gRNA” or “guide RNA” as used herein can refer to guide RNA sequences used to target specific polynucleotide sequences for gene editing employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspect, a gRNA is synthetic (Kelley, M. et al. (2016) J of Biotechnology 233 (2016) 74-83).

“Homology” or “identity” or “similarity” can refer to sequence similarity between two peptides or between two nucleic acid molecules. Homology can be determined by comparing a position in each sequence which can be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

As a practical matter, a particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Lengt, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. Two polynucleotides can be aligned using similar techniques.

“Hybridization” can refer to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein can refer to molecules or biologicals or cellular materials being substantially free from other materials. In one aspect, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation is placed on the maximum number of amino acids which can comprise a protein's or peptide's sequence. As used herein the term “amino acid” can refer to either natural and/or unnatural or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that is used to link these domains together—optionally to preserve the conformation of the fused protein domains and/or prevent unfavorable interactions between the fused protein domains which can compromise their respective functions.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double- and single-stranded molecules. Unless otherwise specified or required, any embodiment of this disclosure that is a polynucleotide encompasses both the double-stranded form and each of two complementary single-stranded forms known or predicted to make up the double-stranded form.

It is understood that the polynucleotides described herein include “genes” and that the nucleic acid molecules described herein include “vectors” or “plasmids.” For example, a polynucleotide encoding a TCF4 can be encoded by an TCF4 gene or homolog thereof. Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence. The term “nucleic acid” or “recombinant nucleic acid” refers to polynucleotides such as deoxyribonucleic acid (DNA), and, where appropriate, ribonucleic acid (RNA). Any sequence comprising thymine (T) as provided herein can be converted to an RNA sequence by replacing the “T” with “U” (uracil). Accordingly, both DNA and RNA sequences are contemplated herein.

Those of skill in the art will recognize that, due to the degenerate nature of the genetic code, a variety of DNA or RNA compounds differing in their nucleotide sequences can be used to encode a given amino acid sequence of the disclosure. The native DNA or RNA sequence encoding TCF4 is only an illustrative embodiment of the disclosure, and the disclosure includes DNA compounds of any sequence that encode the amino acid sequences of the polypeptides and proteins utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate embodiments of the disclosure.

A nucleic acid of the disclosure can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the TCF4 polypeptide described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitutions, in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “polynucleotide sequence” is the alphabetical representation of a polynucleotide molecule. This alphabetical representation can be input into databases in a computer having a central processing unit and used for bioinformatics applications such as functional genomics and homology searching.

As used herein, the term “recombinant expression system” refers to a genetic construct or constructs for the expression of certain genetic material formed by recombination; the term “construct” in this regard is interchangeable with the term “vector” as defined herein.

As used herein the term “restoring” in relation to expression of a protein can refer to the ability to establish expression of full length protein where previously protein expression was truncated due to mutation. In the context of “restoring activity” the term includes effecting the expression of a protein to its normal, non-mutated levels where a mutation resulted in aberrant expression (e.g., too low or too high).

“Transformation” refers to the process by which a vector is introduced into a host cell. Transformation (or transduction, or transfection), can be achieved by any one of a number of means including viral delivery, electroporation, microinjection, biolistics (or particle bombardment-mediated delivery), etc.

As used herein, the terms “treating,” “treatment” and the like are used herein to mean obtaining a desired pharmacologic and/or physiologic effect. The effect can be prophylactic in terms of completely or partially preventing a disease, disorder, or condition or sign or symptom thereof, and/or can be therapeutic in terms of a partial or complete cure for a disorder and/or adverse effect attributable to the disorder.

As used herein, the term “vector” can refer to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct can refer to the polynucleotide comprising the retroviral genome or part thereof, and a gene of interest. Further details as to modern methods of vectors for use in gene transfer can be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

PTHS can be caused by heterozygous mutations in the TCF4 gene, which encodes a basic helix-loop-helix (bHLH) transcription factor. PTHS patients exhibit severe intellectual disability and cognitive impairment, pronounced developmental delay, complete absence of spoken language, and a characteristic facial gestalt. Most patients display hypotonia, motor delay, and/or ataxic gait. A common manifestation is constipation, probably due to enteric nervous system anomalies. Breathing abnormalities and seizures are a variable clinical finding, sometimes of late onset. Autistic behaviors include lack of language communication, intellectual disability, and repetitive self-centered behaviors.

The TCF4 gene is located on chromosome 18 (18q21.2) and encompasses 18 coding exons. Its longest and most extensively studied alternatively spliced transcript encodes the TCF4-B protein isoform, a bHLH transcription factor highly expressed throughout the brain during development. The TCF4 protein binds to E-box regulatory sequences (consensus CANNTG) and has been implicated in numerous developmental processes in the immune system, in epithelial-mesenchymal transition, and in the nervous system. Most PTHS patients display a private mutation in the TCF4 gene, which may be large chromosomal deletions spanning the whole gene, partial gene deletions, translocations, or point mutations.

The disclosure provides DNA constructs and methods for changing the expression of the human gene TCF4 (OMIM 602272; synonyms E2-2, ITF2, PTHS, SEF2, and bHLHb19), and therefore can be used to develop methods for increasing the expression of the TCF4 gene in diseased cells and tissues or in individuals carrying decreased TCF4 expression, such as, but not limited to, human subjects bearing a genetic condition known as Pitt-Hopkins Syndrome (PTHS; MIM #610954). Of interest, TCF4 is also a top risk for Schizophrenia based on WGAS studies.

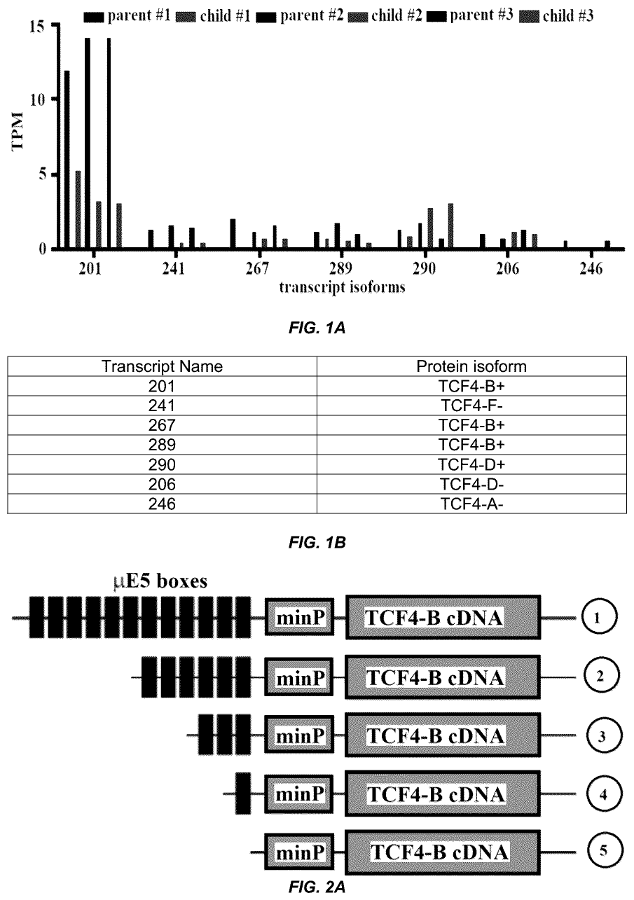

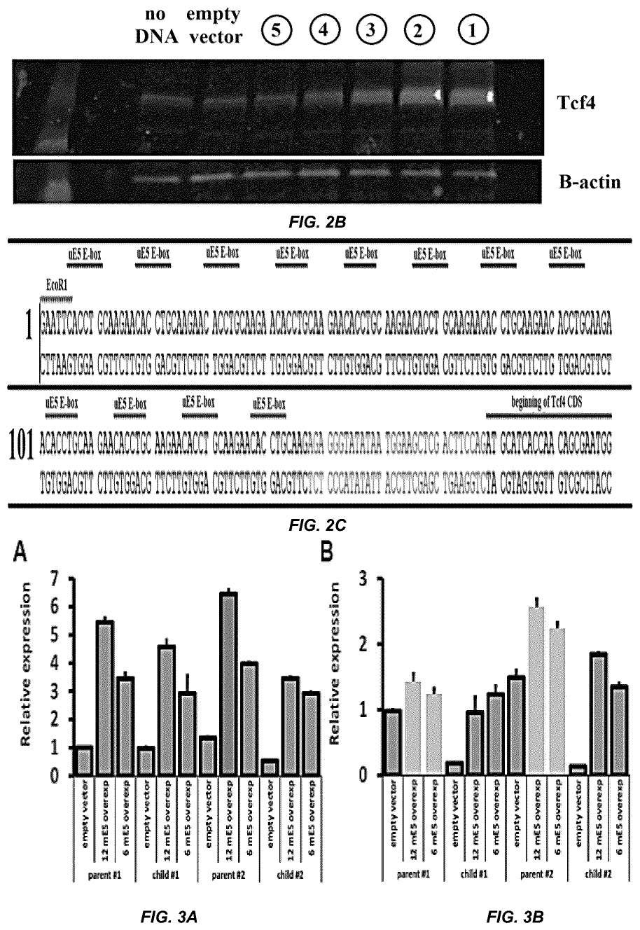

The disclosure provides a plurality of expression cassettes that can be used with suitable DNA constructs and vectors to deliver and/or increase TCF4 or the expression of TCF4. For example, in one embodiment, an extra-copy of the TCF4 coding sequence (e.g., a TCF4 gene) is inserted into target cells or tissues. The DNA constructs comprises the coding sequence of the TCF4-B transcript or variant is typically preceded by DNA regulatory elements that allow control of the expression rate once the construct is inserted in target cells. TCF4-B is used as an example based on its high expression levels in neural progenitor cells and neurons (FIG. 1). In one embodiment, the expression cassette TCF4-B cDNA sequence can be placed under the control of a synthetic minimal promoter (minP) preceded by varying numbers (0, 1, 2, 3, 4, 5, 6, 7, 8, 9, 10, 11, 12, 3, 14, 15) of regulatory sequences recognized by the TCF4 protein itself, known as pE5 boxes (FIG. 2). This approach provides for expression of the TCF4 gene only in cell types where it is normally found.

The results show that the levels of TCF4 expression can be manipulated by changing the number of pE5 boxes, providing controllable overexpression of the TCF4 gene in target cells (FIG. 2). Moreover, these DNA constructs have been used in lentiviral particles to infect (transduce) diseased target cells derived from PTHS patients. In particular, the constructs have been introduced into neural progenitor cells (NPCs). The experiments verified that they can enhance TCF4 expression in these cells (FIG. 3A), increasing its expression level 2 to 5-fold, depending on the number of pE5 boxes in the particular construct tested. Moreover, this genetic manipulation corrected the expression of TCF4 target genes, such as GADD45G (FIG. 3B), bringing its levels back to normal levels seen in control cell lines (FIG. 3B).

As provided herein a cassette of the disclosure can comprise a mini-promoter operably linked to a polynucleotide that is at least 85%, 90%, 92%, 95%, 98%, 99% or 100% identical to a TCF4 (TCF4-B) cDNA:

(SEQ ID NO: 1)
ATGCATCACCAACAGCGAATGGCTGCCTTAGGGACGGACA
AAGAGCTGAGTGATTTACTGGATTTCAGTGCGATGTTTTC
ACCTCCTGTGAGCAGTGGGAAAAATGGACCAACTTCTTTG
GCAAGTGGACATTTTACTGGCTCAAATGTAGAAGACAGAA
GTAGCTCAGGGTCCTGGGGGAATGGAGGACATCCAAGCCC
GTCCAGGAACTATGGAGATGGGACTCCCTATGACCACATG
ACCAGCAGGGACCTTGGGTCACATGACAATCTCTCTCCAC
CTTTTGTCAATTCCAGAATACAAAGTAAAACAGAAAGGGG
CTCATACTCATCTTATGGGAGAGAATCAAACTTACAGGGT
TGCCACCAGCAGAGTCTCCTTGGAGGTGACATGGATATGG
GCAACCCAGGAACCCTTTCGCCCACCAAACCTGGTTCCCA
GTACTATCAGTATTCTAGCAATAATCCCCGAAGGAGGCCT
CTTCACAGTAGTGCCATGGAGGTGCAGACAAAGAAAGTTC
GAAAAGTTCCTCCAGGTTTGCCATCTTCAGTCTATGCTCC
ATCAGCAAGCACTGCCGACTACAATAGGGACTCGCCAGGC
TATCCTTCCTCCAAACCAGCAACCAGCACTTTCCCTAGCT
CCTTCTTCATGCAAGATGGCCATCACAGCAGTGACCCTTG
GAGCTCCTCCAGTGGGATGAATCAGCCTGGCTATGCAGGA
ATGTTGGGCAACTCTTCTCATATTCCACAGTCCAGCAGCT
ACTGTAGCCTGCATCCACATGAACGTTTGAGCTATCCATC
ACACTCCTCAGCAGACATCAATTCCAGTCTTCCTCCGATG
TCCACTTTCCATCGTAGTGGTACAAACCATTACAGCACCT
CTTCCTGTACGCCTCCTGCCAACGGGACAGACAGTATAAT
GGCAAATAGAGGAAGCGGGGCAGCCGGCAGCTCCCAGACT
GGAGATGCTCTGGGGAAAGCACTTGCTTCGATCTATTCTC
CAGATCACACTAACAACAGCTTTTCATCAAACCCTTCAAC
TCCTGTTGGCTCTCCTCCATCTCTCTCAGCAGGCACAGCT
GTTTGGTCTAGAAATGGAGGACAGGCCTCATCGTCTCCTA
ATTATGAAGGACCCTTACACTCTTTGCAAAGCCGAATTGA
AGATCGTTTAGAAAGACTGGATGATGCTATTCATGTTCTC
CGGAACCATGCAGTGGGCCCATCCACAGCTATGCCTGGTG
GTCATGGGGACATGCATGGAATCATTGGACCTTCTCATAA
TGGAGCCATGGGTGGTCTGGGCTCAGGGTATGGAACCGGC
CTTCTTTCAGCCAACAGACATTCACTCATGGTGGGGACCC
ATCGTGAAGATGGCGTGGCCCTGAGAGGCAGCCATTCTCT
TCTGCCAAACCAGGTTCCGGTTCCACAGCTTCCTGTCCAG
TCTGCGACTTCCCCTGACCTGAACCCACCCCAGGACCCTT
ACAGAGGCATGCCACCAGGACTACAGGGGCAGAGTGTCTC
CTCTGGCAGCTCTGAGATCAAATCCGATGACGAGGGTGAT
GAGAACCTGCAAGACACGAAATCTTCGGAGGACAAGAAAT
TAGATGACGACAAGAAGGATATCAAATCAATTACTAGGTC
AAGATCTAGCAATAATGACGATGAGGACCTGACACCAGAG
CAGAAGGCAGAGCGTGAGAAGGAGCGGAGGATGGCCAACA
ATGCCCGAGAGCGTCTGCGGGTCCGTGACATCAACGAGGC
TTTCAAAGAGCTCGGCCGCATGGTGCAGCTCCACCTCAAG
AGTGACAAGCCCCAGACCAAGCTCCTGATCCTCCACCAGG
CGGTGGCCGTCATCCTCAGTCTGGAGCAGCAAGTCCGAGA
AAGGAATCTGAATCCGAAAGCTGCGTGTCTGAAAAGAAGG
GAGGAAGAGAAGGTGTCCTCAGAGCCTCCCCCTCTCTCCT
TGGCCGGCCCACACCCTGGAATGGGAGACGCATCGAATCA
CATGGGACAGATGTAA

and encodes a polypeptide of SEQ ID NO:2:

MHHQQRMAALGTDKELSDLLDFSAMFSPPVSSGKNGPTSL
ASGHFTGSNVEDRSSSGSWGNGGHPSPSRNYGDGTPYDHM
TSRDLGSHDNLSPPFVNSRIQSKTERGSYSSYGRESNLQG
CHQQSLLGGDMDMGNPGTLSPTKPGSQYYQYSSNNPRRRP
LHSSAMEVQTKKVRKVPPGLPSSVYAPSASTADYNRDSPG
YPSSKPATSTFPSSFFMQDGHHSSDPWSSSSGMNQPGYAG
MLGNSSHIPQSSSYCSLHPHERLSYPSHHTNNSFSSNPST
PVGSPPSLSAGTAVWSRNGGQASSSPNYEGPLHSLQSRIE
DRLERLDDAIHVLRNHAVGPSTAMPGGHGDMHGIIGPSHN
GAMGGLGSGYGTGLLSANRHSLMVGTHREDGVALRGSHSL
LPNQVPVPQLPVQSATSPDLNPPQDPYRGMPPGLQGQSVS
SGSSEIKSDDEGDENLQDTKSSEDKKLDDDKKDIKSITRS
RSSNNDDEDLTPEQKAEREKERRMANNARERLRVRDINEA
FKELGRMVQLHLKSDKPQTKLLILHQAVAVILSLEQQVRE
RNLNPKAACLKRREEEKVSSEPPPLSLAGPHPGMGDASNH
MGQM

The cassette can comprise a minimal promoter and one or more (e.g., 1, 2, 3, 4, 5, 6, 7, 8, 9, 10, 11, 12, 13, 14, 15 or more) protein binding domains such as one or more pE5 box domains operably linked to the minimal promoter. A pE5 box has the sequence: cacctg; and in some embodiments, the pE5 is spaced from the next adjacent pE5 by about 3 to about 10 nucleotides, such as from about 4 to about 8 nucleotide (e.g., about 6 nucleotides). such that it can comprise a sequence of SEQ ID NO:10). An exemplary pE5 is represented by SEQ ID NO:10. An exemplary minimal promoter includes agagggtatataatggaagctcgacttccag (SEQ ID NO:3). Other minimal promoters or core promoters are described herein. In some embodiments, the minimal promoter of SEQ ID NO:3 is separated from pE5 domain by spacers (e.g., caagaa).

The cassette can be positioned into a suitable vector by recombinant molecular biology techniques to promote delivery to a cell. As mentioned below, the suitable vector can be a DNA construct or a viral vector (e.g., adeno-viral vectors, retroviral vectors such lentiviral vectors and gamma viral vectors).

Most promoters are rather large; typically over 600 bp and a full sized promoter can be many kilobases. Smaller promoters can be generated that allow reliable expression of transgenes in mammalian cells from vectors such as retroviral vectors including replicating and non-replicating viral vectors. As mentioned above a suitable minipromoter can comprise SEQ ID NO:3. Other suitable minimporomoters can be derived from “core” promoters described by Kadanaga and collaborators (Juven-Gershon et al., Nature Methods, 11:917-922, 2006). These core promoters are based on the adenovirus major late (AdML) and cytomegalovirus (CMV) major immediate early genes, and the synthetic “super core promoter”-1 (SCP1). Other cellular core promoters include, but are not limited to, the human home oxygenase proximal promoter (121 bp; Tyrrell et al., Carcinogenesis, 14: 761-765, 1993), the CTP:phosphocholine cytidylyltransferase (CCT) promoter (240 bp; Zhou et al., Am. J. Respir. Cell Mol. Biol., 30: 61-68, 2004); the Human ASK (for Activator of S phase Kinase, also known as HsDbf4 gene, 63 bp; Yamada et al. J. Biol. Chem., 277: 27668-27681, 2002); and the HSVTK intragenic core (Al-Shawi et al., Mol. Cell. Biol., 11: 4207, 1991; Salamon et al., Mol. Cell. Biol., 15:5322, 1995). Furthermore, these “core” promoters can be used as a starting point for further modifications to improve the activity of the promoter. For example, such modifications including the addition of other domains and sequences to the “core” promoter to improve functionality (e.g., enhancers, Kozak sequences and the like). In one embodiment, such further modifications can include the addition of enhancers or transcription binding protein sequence.

The length of these core promoters are approximately 30-80 bp each, thus, when used in a viral vector provide ample additional capacity for transgene sequence. The use of such promoters can give useful expression of genes such as a TCF4 gene or coding sequence (e.g., SEQ ID NO:1).

Furthermore, using rational design techniques various promoter-components can be used to optimize expression and stability of vectors and cassettes. Such optimized core promoters provide a more effective expression and stability of the viral polynucleotide. For example, “designer” promoters can comprise a core promoter that has been further modified to include one or more additional elements suitable for stability and expression.

As used herein, a “core promoter” refers to a minimal promoter comprising about 30-100 bp and lacks enhancer elements. Such core promoters include, but are not limited to, SCP1, AdML and CMV core promoters and the promoter of SEQ ID NO:3. An exemplary promoter can comprise SEQ ID NO:3.

Core promoters include certain viral promoters. Viral promoters, as used herein, are promoters that have a core sequence but also usually some further accessory elements. For example, the early promoter for SV40 contains three types of elements: a TATA box, an initiation site and a GC repeat (Barrera-Saldana et al., EMBO J, 4:3839-3849, 1985; Yaniv, Virology, 384:369-374, 2009). The TATA box is located approximately 20 base-pairs upstream from the transcriptional start site. The GC repeat regions is a 21 base-pair repeat containing six GC boxes and is the site that determines the direction of transcription. This core promoter sequence is around 100 bp. Adding an additional 72 base-pair repeats, thus making it a “small-promoter,” is useful as a transcriptional enhancer that increase the functionality of the promoter by a factor of about 10. When the SP1 protein interacts with the 21 bp repeats it binds either the first or the last three GC boxes. Binding of the first three initiates early expression, and binding of the last three initiates late expression. The function of the 72 bp repeats is to enhance the amount of stable RNA and increase the rate of synthesis. This is done by binding (dimerization) with the AP1 (activator protein 1) to give a primary transcript that is 3′ polyadenylated and 5′ capped. Other viral promoters, such as the Rous Sarcom Virus (RSV), the HBV X gene promoter, and the Herpes Thymidine kinase core promoter can also be used as the basis for selection desired function.

A core promoter typically encompasses −40 to +40 relative to the +1 transcription start site (Juven-Gershon and Kadonaga, Dev. Biol. 339:225-229, 2010), which defines the location at which the RNA polymerase II machinery initiates transcription. Typically, RNA polymerase II interacts with a number of transcription factors that bind to DNA motifs in the promoter. These factors are commonly known as “general” or “basal” transcriptions factors and include, but are not limited to, TFIIA (transcription factor for RNA polymerase IIA), TFIIB, TFIID, TFIIE, TFIIF, and TFIIH. These factors act in a “general” manner with all core promoters; hence they are often referred to as the “basal” transcription factors.

Juven-Gershon et al., 2006 (supra), describe elements of core promoters. For example, the pRC/CMV core promoter consists of a TATA box and is 81 bp in length; the CMV core promoter consists of a TATA box and a initiator site; while the SCP synthetic core promoters (SCP1 and SCP2) consist of a TATA box, an Inr (initiator), an MTE site (Motif Ten Element), and a DPE site (Down stream promoter element) and is about 81 bp in length. The SCP synthetic promoter has improved expression compared to the simple pRC/CMV core promoter.

As used herein a “mini-promoter” or “small promoter” refers to a regulatory domain that promotes transcription of an operably linked gene or coding nucleic acid sequence. The mini-promoter, as the name implies, includes the minimal amount of elements necessary for effective transcription and/or translation of an operably linked coding sequence. A mini-promoter can comprise a “core promoter” in combination with additional regulatory elements or a “modified core promoter”. Typically, the mini-promoter or modified core promoter will be about 30-600 bp in length while a core promoter is typically less than about 100 bp (e.g., about 30-80 bp). In other embodiments, where a core promoter is present, the cassette can optionally comprise an enhancer element or another element either upstream or downstream of the core promoter sequence that facilitates expression of an operably linked coding sequence above the expression levels of the core promoter alone.

Accordingly, the disclosure provides mini-promoters (e.g., modified core promoters) derived from cellular elements as determined for “core promoter” elements that allow ubiquitous expression at significant levels in target cells and are useful for stable incorporation into vectors, in general, and viral vectors, in particular, to allow efficient expression of transgenes. Also provided are mini-promoters comprising core promoters plus minimal enhancer sequences and/or Kozak sequences to allow better gene expression compared to a core-promoter lacking such sequences that are still under 200, 400 or 600 bp. Such mini-promoters include modified core promoters and naturally occurring tissue specific promoters such as the elastin promoter (specific for pancreatic acinar cells, (204 bp; Hammer et al., Mol Cell Biol., 7:2956-2967, 1987) and the promoter from the cell cycle dependent ASK gene from mouse and man (63-380 bp; Yamada et al., J. Biol. Chem., 277: 27668-27681, 2002). Ubiquitously expressed small promoters also include viral promoters such as the SV40 early and late promoters (about 340 bp), the RSV LTR promoter (about 270 bp) and the HBV X gene promoter (about 180 bp) (e.g., R Anish et al., PLoS One, 4: 5103, 2009) that has no canonical “TATTAA box” and has a 13 bp core sequence of 5′-CCCCGTTGCCCGG-3′.

As described herein, the use of such mini-promoters either alone or including additional elements for expression can be used in various cassettes and vectors including replication competent and incompetent viral vectors to express a TCF4 coding sequence (e.g., SEQ ID NO:1). For example, the disclosure provides a cassette that can be incorporated into an expression vector or viral vectors. Various vectors are known and the cloning of the cassette into such expression vectors or viral vectors can be performed. For example, some viral vectors tolerate cloning of a cassette into the long terminal repeats (LTRs). Other vectors tolerate cloning of the cassette downstream of the envelope gene, but upstream of the 3′ LTR. Yet other non-replicating vectors have greater cassette capacity as they have had key genes removes (e.g., gag and pol).

Another suitable delivery vehicle for the CNS comprises nanoparticles, typically having a size of less than 200 nm, or less than about 150 nm, or less than about 100 nm. These may include lipid-based nanoparticles, polymer nanoparticles, dendrimers and inorganic nanoparticles, some of which may be tailored to pass through the blood brain barrier (BBB). In some embodiments, the delivery system actively targets delivery by using ligands of transporters or receptors to enhance nanoparticle uptake across the BBB. The preferred pathway for this approach is receptor (or transporter)-mediated transcytosis by which a cargo (e.g., nanoparticles) transports between the apical and basolateral surface in the brain ECs. For example, low-density lipoproteins undergo transcytosis through the ECs by a receptor-mediated process, bypassing the lysosomal compartment and releasing at the basolateral surface of the brain side. Further, since the BBB contains transporters to amino acids, using the naturally present arginine transporter for the delivery is one approach for delivery to the brain. Another vehicle for brain delivery is exosomes which are small extracellular vesicles secreted by cells. The major advantage of exosomes versus other synthetic nanoparticles is their non-immunogenic nature, leading to a long and stable circulation.

The disclosure provides methods and compositions for treating and/or reducing the symptoms of a neurological or neurodevelopmental disease and disorder that is associated with the aberrant expression of TCF4 in neuronal cells of the central nervous system (CNS), or peripheral nervous system (PNS), by administering an effective amount of a construct comprising a TCF4 cassette of the disclosure such that the cassette is expressed by the neuronal cell. The neurological or neurodevelopmental disease or disorder may also be associated with defective or abnormal TCF4 transcription factor gene expression and/or protein function in the neuronal cells, e.g., through mutation or haploinsufficiency. Such neurological or neurodevelopmental diseases and disorders encompass, for example, Pitt-Hopkins Syndrome (PTHS), schizophrenia, autism, autism spectrum disorder, etc. A TCF4 cassette can be designed such that a construct comprising the cassette is ectopically expressed in the neuronal cells. As used herein, ectopic expression refers to the expression and/or activity of protein in cells and/or tissues in which it is not normally expressed. In the instant case, aberrant, abnormal, or atypical expression or activity of a TCF4.

The disclosure provides a method of treating and/or reducing the symptoms of a neurological or neurodevelopmental disease or disorder comprising delivering a TCF4 cassette and expressing the cassette to treat and/or reduce the symptoms of the neurological or neurodevelopmental disease or disorder. In one embodiment, a vector comprising a TCF4 cassette is an AAV9 vector having a sequence of SEQ ID NO:9 or a sequence that is at least 80%, 85%, 90%, 92%, 95%, 97%, 98% or 99% identical to SEQ ID NO:9. In an embodiment, the neurological or neurodevelopmental disease or disorder is associated with defective or abnormal TCF4 transcription factor gene expression and/or protein function in the neuronal cells. In an embodiment, the subject in need has, is suspected of having, or is at risk of (for example, has been identified as having a mutation in TCF4) having such a neurological or neurodevelopmental disease or disorder. In embodiments, the neurological or neurodevelopmental disease or disorder is Pitt-Hopkins Syndrome, schizophrenia, autism, autism spectrum disorder, or 18q syndrome, etc.

The disclosure also provides a method of treating and/or reducing the symptoms of a neurological or neurodevelopmental disease or disorder that is associated with abnormal or defective neuronal TCF4 expression and/or function, in a subject in need, by administering to the subject a therapeutically effective amount of a TCF4 construct of the disclosure (e.g., a vector comprising a TCF4 cassette).

The disclosure also provides pharmaceutical compositions for the administration of a vector and/or cassette of the disclosure can be conveniently presented in dosage unit form and can be prepared by any of the methods well known in the art of pharmacy. The pharmaceutical compositions can be, for example, prepared by uniformly and intimately bringing the vector and/or a cassette-containing composition provided herein into association with a liquid carrier, a finely divided solid carrier or both. In the pharmaceutical composition the compound provided herein is included in an amount sufficient to produce the desired therapeutic effect.

Systemic formulations include those designed for administration by injection (e.g., subcutaneous, intravenous, infusion, intramuscular, intracerebral, intraspinal, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions can also contain formulating agents, such as suspending, stabilizing, and/or dispersing agents. The formulations for injection can be presented in unit dosage form, e.g., in ampules or in multidose containers, and can contain added preservatives.

Alternatively, the injectable formulation can be provided in powder form for reconstitution with a suitable vehicle, including but not limited to sterile pyrogen free water, buffer, and dextrose solution, before use. To this end, the composition provided herein can be dried by any art-known technique, such as lyophilization, and reconstituted prior to use.

“Administration” can be effected in one dose, continuously or intermittently throughout the course of treatment. Methods of determining the most effective means and dosage of administration are known to those of skill in the art and can vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and can vary with the composition used for treatment, the purpose of the treatment, the health condition or disease stage of the subject being treated, and target cell or tissue.

Administration can refer to methods that can be used to enable delivery of compounds or compositions to the desired site of biological action (such a DNA constructs, viral vectors, or others). These methods can include parenteral administration (including intravenous, subcutaneous, intrathecal, intraperitoneal, intramuscular, intravascular or infusion), intracerebral and intraspinal. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, a cosmetic professional can administer the composition.

Administration or application of a composition disclosed herein can be performed for a treatment duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of composition disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered/applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

A “composition” typically intends a combination of the active agent, e.g., a TCF4 cassette of this disclosure, typically in a vector such as a viral vector (e.g., and AAV9 vector), and a naturally-occurring or non-naturally-occurring carrier, inert or active, such as an adjuvant, diluent, binder, stabilizer, buffers, salts, lipophilic solvents, preservative, adjuvant or the like and include pharmaceutically acceptable carriers. In one embodiment, the composition comprises a sequence that is at least 80%-100% identical to SEQ ID NO:9. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, and pharmaceutical formulations can be packaged in dosage unit form for ease of administration and uniformity of dosage. The term “unit dose” or “dosage” can refer to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions can be administered in a manner compatible with the dosage formulation and in such amount as is therapeutically or prophylactically effective. The formulations are easily administered in a variety of dosage forms, such as the type of injectable solutions described herein.

### Examples

Human subjects. Subjects are members of volunteering families recruited through the Pitt Hopkins Research Foundation. PTHS subjects (Table 1) were selected based on availability of detailed clinical and molecular diagnostics information, including the types of TCF4 mutation they carry. For patients harboring a point mutation, small indel, or translocation, the details of each TCF4 mutation was confirmed via resequencing of the TCF4 locus. A detailed and personalized questionnaire to gather information related to the patients' PTHS clinical symptoms was answered by all participating families, encompassing questions about neurological findings, cognitive, behavioral and gastroenterological manifestations, age at diagnosis, general quality of life, temporal evolution of motor milestones, communication level, dysmorphic facial features, urological symptoms, vision problems, sensory responsivity, sleep disturbances, respiratory anomalies such as apnea and hyperventilation, feeding habits and bowel symptoms, history of seizures, as well as MRI findings. These data are reported in Table 1. To maximize comparability, only male subjects were selected for this study. Control subjects were the patients' corresponding fathers, who had no history of psychiatric or genetic disorders. The participation of all subjects was approved by the Human Subjects Ethics Committees of the institutes in which the study was conducted. Written informed consent was obtained from all participating families after receiving a thorough description of the study. It is important to note that TCF4 (Transcript ion Factor 4) should not be confused with TCF-4 (T-Cell factor 4), an old and outdated name for TCPL7, one of the TCF-LEF proteins, the endpoints of the Wnt signaling pathway and totally unrelated to the TCF4 mutated in PTHS patients.

TABLE 1
Summary of participating subjects and clinical
characteristics. Related to all figures.

| | symbol | | | type | PTHS | |
|---|---|---|---|---|---|---|
| | in | | | of TCF4 | clinical | |
| patient | figures | gender | age | mutation | features | abMRI |
| PTHS #1 | ♦ | M | 14 | small insertion | FG MM CT | yes |
| | | | | (c.1067_1068insTC) | SZ BA VA | |
| PTHS #2 | ▪ | M | 8 | whole gene deletion | FG MM AS | yes |
| | | | | (del 6.6 q21.2-q21.33) | CT VA RB | |
| PTHS #3 | ▴ | M | 10 | translocation [t(2; 18) | FG SM AS | no |
| | | | | (chr2: 197, 989, 212- | CT VA RB | |
| | | | | 197, 989, 746_chr18: 52, | | |
| | | | | 895, 297-52, 895, 597)] | | |
| PTHS #4 | ● | M | 4 | point mutation (c.959 | FG SM AS | no |
| | | | | C > T; Thr320Ile) | CT VA | |
| PTHS #5 | + | M | 7 | partial gene deletion | FG SM AS | yes |
| | | | | (c.454_1072del) | SZ RB | |
| PTHS #6 | | F | 7 | — | | |
| parent #1 | ♦ | M | 40 | — | — | — |
| parent #2 | ▪ | M | 30 | — | — | — |
| parent #3 | ▴ | M | 50 | — | — | — |
| parent #4 | ● | M | 44 | — | — | — |
| parent #5 | + | M | 45 | — | — | — |

For all patients, controls are parents of matching sex.
Abbreviations:
FG: dysmorphic facial gestalt;
SM/MM: severe (SM) or mild (MM) motor delay at age 3;
AS: absent speech;
CT: constipation;
SZ: seizures;
BA: breathing problems (hyperventilation or apnea);
UA: urinary abnormalities (retention or incontinence);
VA: visual abnormalities (ocular anomalies);
RB: repetitive behaviors;
abMRI: brain anomalies (thinned corpus callosum) detected by MRI.

Reprogramming of skin fibroblasts into induced pluripotent stem cells (iPSCs). Skin fibroblasts were obtained from biopsies taken from PTHS and control subjects, followed by culturing in DMEM/F12 medium containing 103 fetal bovine serum and penicillin/streptomycin. iPSCs were derived from fibroblasts via cellular reprogramming, as described in (Marchetto et al., 2017). Briefly, fibroblast cultures were transduced with Sendai viruses containing over-expression cassettes for OCT4, SOX2, RLF4, and MYC (Cytotune iPS 2.0 Sendai reprogramming kit; Thermo Fisher Scientific). Seven days after transduction, cells were re-plated onto a feeding layer composed of murine embryonic fibroblasts (mEFs) in DMEM/F12 containing 20% Knock-out Serum Replacement (Thermo Fisher Scientific), 1% non-essential amino acids (NEAA), and 100 μM β-mercaptoethanol. iPSC colonies were identified after 2 weeks and transferred to 6 cm plates coated with Matrigelk (BD Biosciences), after which time they were maintained in mTeSR1 medium (StemCell Technologies) and passaged by manual picking with the aid of a pipette tip. A total of 20 iPSC lines were produced for each subject in the study, all of which were analyzed through a combination of immunostaining and SNP mapping to rule out the presence of unwanted chromosomal abnormalities and mutations (example in FIG. 12B). All iPSC clones were passaged until P10 and 2 clones were chosen for further NPC and organoid derivation after this passage. Most experiments in this study were conducted with one P15 iPSC clone per subject. Cultures were tested every two weeks for mycoplasma, and contamination was never identified at any stage.

Validation of iPSC was performed by immunostaining for SOX2, OCT4, NANOG, and LIN28. Briefly, a total of 20 colonies were grown inside wells of LabTek II 8-well chambered slides (Thermo Fisher Scientific) until they reached a size of 2 mm. Colonies were then fixed with 4 paraformaldehyde solution for 10 min, washed once with 1× Phosphate Buffered Saline (PBS), permeabilized with 1% Triton X-100 for 5 min, washed again in 1×PBS, and blocked with Bovine Serum Albumin (BSA)/1t Triton X-100/1×PBS. Incubation with primary antibodies was performed in the same blocking solution for 16 h at 4° C. Primary antibodies used were anti-SOX2 (Abcam; ab97959), anti-OCT4 (Abcam; ab19857), anti-NANOG (GeneTex; GTX100863), and anti-LIN28 (Cell Signaling; 3978). After 3 washes in 1×PBS, colonies were incubated with fluorescently labeled secondary antibodies for 3 h, and nuclei were counterstained with 1 μg/mL DAPI (Thermo Fisher Scientific) for 30 min. Slides were mounted with ProLong Gold anti-fading mountant (Thermo Fisher Scientific).

For identifying unwanted chromosomal structural alterations, genome-wide profiling for amplifications, deletions, copy number variation, and rearrangements was performed on genomic DNA extracted from the iPSC lines using the iScan system (Illumina) and the Infinium HumanCytoSNP-12 BeadChip (Illumina; 299,140 genetic markers). Clones containing visibly large deletions and duplications were not found. An example of karyotyping conducted using this technique is presented in FIG. 12B for PTHS patient #2, showing the expected large deletion in the long arm of chromosome 18 for this patient line.

Pallial and subpallial organoid generation. For the generation of pallial (cortical) brain organoids (CtOs), iPSC colonies were dissociated using Accutase (Thermo Fisher Scientific; diluted with an equal volume of 1×PBS) for 12 minutes at 37° C. After centrifugation for 3 min at 150×g, the individualized cells were resuspended in mTeSR1 medium (StemCell Technologies) supplemented with 10 mM SB431542 (Stemgent) and 1 mM dorsomorphin (R&D Systems). Approximately 3-4 million cells were seeded onto each well of a low-binding 6-well plate and placed on a shaker inside a CO₂ incubator at 95 rpm. During the first 24 h, medium was supplemented with 5 mM Rho kinase inhibitor (Y-27632; Calbiochem, Sigma-Aldrich). Over three days, cells clustered to form spherical embryoid bodies, after which time mTeSR1 was replaced with neural induction medium consisting of Neurobasal medium (Thermo Fisher Scientific) containing GlutaMAX, 1% Gem21 NeuroPlex supplement (Gemini Bio-Products), 1% N2 NeuroPlex (Gemini Bio-Products), 1% NEAA (Thermo Fisher Scientific), 1% penicillin/streptomycin (Thermo Fisher Scientific), 10 mM SB431542, and 1 mM dorsomorphin, for 7 days. Next, the medium was replaced with NPC proliferation medium, consisting of Neurobasal medium containing GlutaMAX, 1% Gem21, 1% NEAA, 20 ng/mL FGF-2 (Thermo Fisher Scientific) for 7 days, followed by 7 additional days in the same medium further supplemented with 20 ng/mL EGF (PeproTech). Neuronal differentiation and organoid maturation were achieved by switching to Neurobasal medium containing 1% GlutaMAX, le Gem21, 1% NEAA, 10 ng/mL of BDNF, 10 ng/mL of GDNF, 10 ng/mL of NT-3 (all from PeproTech), 200 mM L-ascorbic acid, and 1 mM dibutyryl-cAMP (Sigma-Aldrich), for 7 days. After this period, CtOs were maintained in Neurobasal medium containing GlutaMAX, 1% Gem21, 1% NEAA for as long as needed, with media changes every 3-4 days. For every subject, most experiments were conducted with at least 3 independent batches (usually more than 10 batches), which were considered independent biological replicates in figures throughout the study, with at least 3 technical replicates (wells of organoids) per batch. For phenotypic evaluations conducted on 4 or more separate batches, two or more independent clones of iPSCs were used to produce the organoids (and NPCs) and to confirm the effect of genotype, as depicted in FIG. 12F.

For the generation of subpallial organoids (sPOs), the protocol previously published in (Birey et al., 2017) was used, with some modifications. After culturing embryoid bodies in mTeSR1 for three days, they were transferred to neural induction medium (Neurobasal medium supplemented with 1% GlutaMAX, 1% Gem21, 1% N2, 1% NEAA, 1% penicillin/streptomycin, 10 mM SB431542, and 1 mM dorsomorphin) containing 5 μM Wnt pathway inhibitor IWP-2 (SelleckChem), from day 4 until day 10. Next, the medium was replaced with NPC proliferation medium, consisting of Neurobasal medium containing 1% GlutaMAX, 1% Gem21, 1% NEAA, 20 ng/mL FGF-2, and 100 nM SHH pathway agonist SAG (SelleckChem) for 7 days, followed by 2 additional days in the same medium supplemented with 20 ng/mL EGF (PeproTech). Culturing for an additional 5 days in the same medium, but without SAG, completed the NPC proliferation phase. This was followed by neuronal differentiation and organoid maturation phases, which were conducted using the same types of medium and durations used in the CtO derivation protocol.

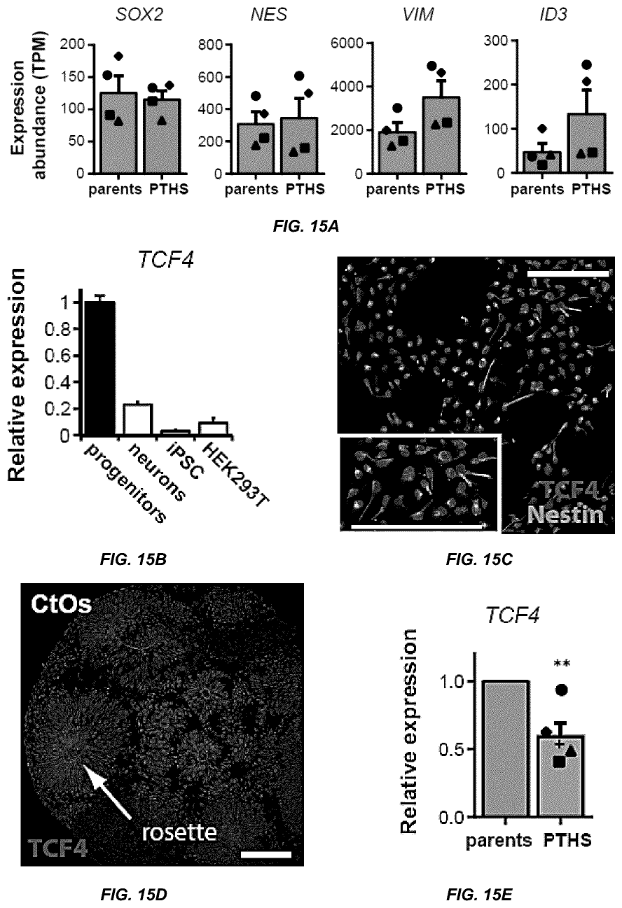

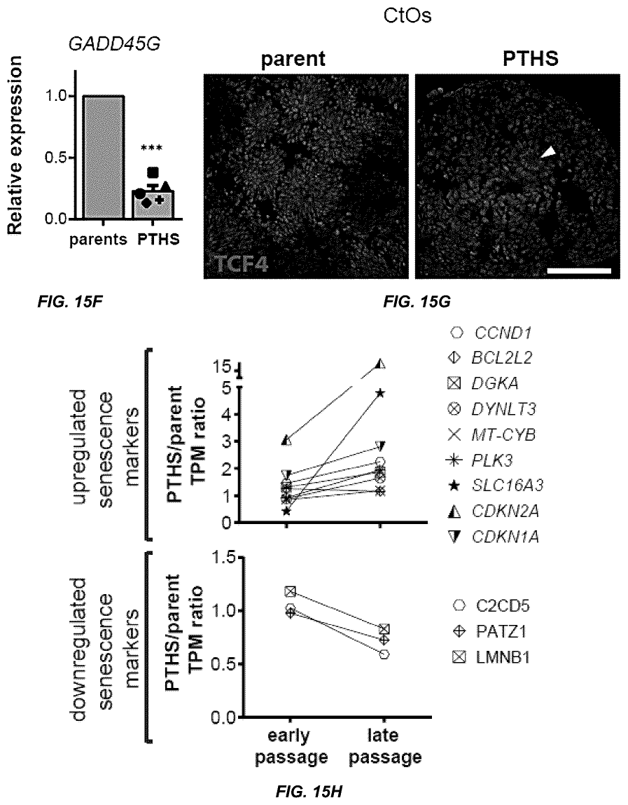

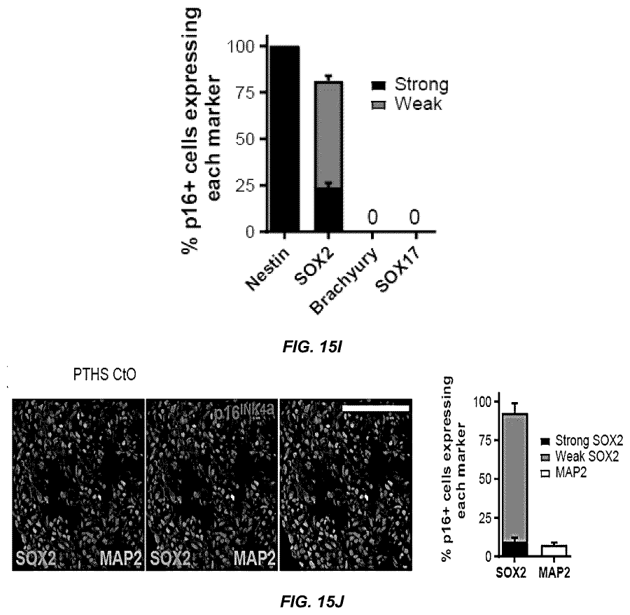

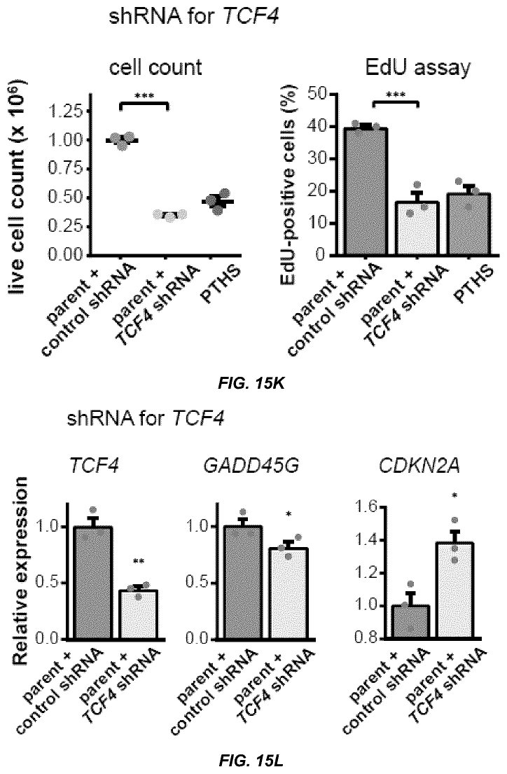

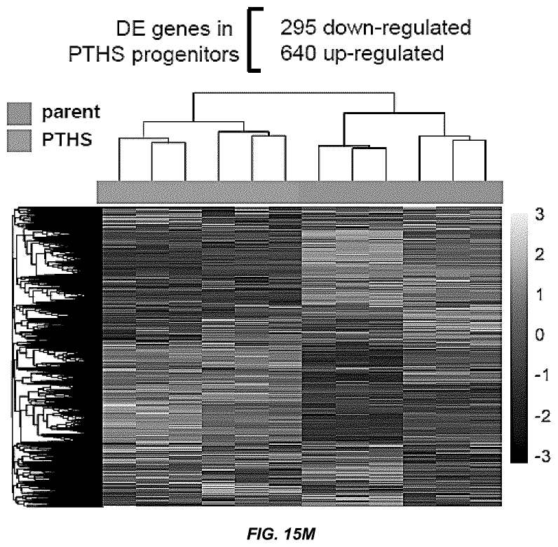

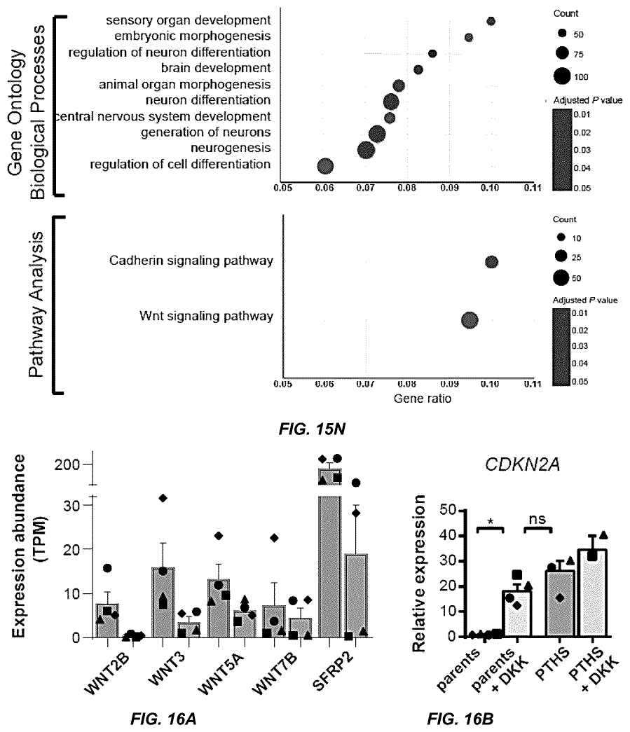

Immunofluorescence staining. After being cultured in vitro for the required amount of time, CtOs and sPOs were fixed with 4% paraformaldehyde for 4-8 h at 4° C. and cryoprotected in 30% sucrose for 12 h. Organoids were then embedded in TissueTek (Leica Microsystems) and sectioned on a Leica VT1000S cryostat to produce 20 μm sections. For staining, slides were air dried for 10 min, permeabilized in 1 Triton X-100/1×PBS for 2 min, and blocked with 0.13 Triton X-100/3% BSA/1×PBS for 1 h at 25° C., followed by incubation with primary antibodies in the same solutions, for 16 h at 4° C. Primary antibodies used were: rat anti-CTIP2 (Abcam; ab18465; 1:500); rabbit anti-SATB2 (Abcam; ab34735; 1:200); chicken anti-MAP2 (Abcam; ab5392; 1:1000); rabbit anti-SOX2 (Cell Signaling Technology; 2748; 1:500); rabbit anti-GAD65/67 (Abcam; ab11070; 1:200); rabbit anti-CUX1 (CUTL1 or CASP) (Abcam; ab54583; 1:200); rabbit anti-TCF4 (Abcam; ab217668; 1:1000); rabbit anti-vGLUT1 (Synaptic Systems; 135311; 1:500); rabbit anti-CC3 (Cleaved Caspase 3) (Cell Signaling; 9664S; 1:500); rabbit anti-doublecortin (DCX) (Abcam; ab18723; 1:200); mouse anti-Cas9 (Abcam; ab210571; 1:200); mouse anti-p16^{INK4a} (CDKN2A) (Abcam; ab54210; 1:1000); rabbit anti-SOX3 (Abcam; ab183606; 1:200); mouse anti-Nestin (Abcam; ab22035; 1:1000); goat anti-SOX17 (R&D Systems; AF1924; 1:200); rabbit anti-Brachyury (Sigma; B8436; 1:200); or rabbit anti-D-catenin (Cell Signaling; 9582S; 1:100). After incubation in a solution containing primary antibodies, slides were washed three times in 1×PBS, for 5 min each, and incubated with fluorescently labeled secondary antibodies (Alexa Fluor 488- or 555-conjugated antibodies; 1:500 dilution; Thermo Fisher Scientific) in the same type of solution as primary antibodies, for 3 h at 25° C. After further washes in 1×PBS, slides were counterstained with DAPI solution (1 μg/mL) for 45 min and mounted with ProLong Gold. All images were taken using a Zeiss fluorescence microscope equipped with Apotome (Axio Observer Apotome, Zeiss). For projections of z series stacked images of DCX-stained organoids, the maximum intensity feature of ZEN software (Zeiss) was used, after collecting 10 optical slices per section. For p16^{INK4a} staining, antigen retrieval was performed by incubating the slides at 60° C. for 10 min in 1× Universal HIER antigen retrieval reagent (Abcam; ab208572), followed by regular immunostaining. For counting SOX2+ cells after p16^{INK4a} co-staining (FIG. 15J), raw unprocessed images were used, and defined strongly stained cells as those which had average pixel intensity between the upper third quartile and the maximum pixel intensity in each image. Remaining SOX2+ cells were considered weakly stained.

For quantification of cell types in organoid sections, 4 random 100×100 μm regions of interest (ROI) were sampled across each imaged section. The mean number of labeled cells per sample was calculated by first averaging the number of labeled cells in each ROI to produce a mean value of labeled cells per section, and then averaging these mean values across all sections for each subject. The number of subjects and sections quantified are indicated in the figure legends. Because vGLUT1 is mostly found outside cell bodies, vGLUT1 and GAD65/67 were quantified (FIG. 12M) by counting pixels in raw unprocessed fluorescence microscopy images using the Color Pixel Counter plugin on the ImageJ software, counting particles of size 1 pixel and color intensity above 50 (in a range from 0 to 255). The average percentage of pixels according to these rules was computed over four 100×100 μm ROIs per section and 6 sections per subject.

For immunofluorescence labeling of NPCs, these cells were seeded at a density of 50,000 cells per well of a LabTek II 8-well chambered slide. When cells reached 50% confluency, they were fixed and processed for immunostaining in the same manner as described for iPSC colonies, with the following primary antibodies: rabbit anti-TCF4 (Abcam; ab217668; 1:1000); and chicken anti-vimentin (VIM) (Abcam; ab22651; 1:2000). NPCs were also stained to detect senescence associated P-galactosidase (SA-P-gal), using the CellEvent™ Senescence Green Detection Kit (Thermo Fisher Scientific; C10850) after antigen retrieval as described above. The same method described above for counting weakly and strongly stained SOX2+ cells after p16^{INK4a} co-staining was applied to NPCs.

Post-mortem brain sample collection and analysis. Patient #6 (Table 1) died at age 7 during a surgical procedure to correct scoliosis, due to complications unrelated to the PTHS neurological symptoms. The hospital pathologists immediately dissected the brain and harvested cortical tissue encompassing the entire width of the cortex at the boundary between the pre-motor and prefrontal areas. Hippocampus tissue was also harvested but is not described in this study. Brain tissue was fixed for 24h under formalin, then fixed in 4% paraformaldehyde for 6h, prior to being cryoprotected in 20% sucrose and sectioned under a vibratome followed by immunostaining as described above. PTHS images were compared with those obtained from sections stained in parallel (FIG. 10E) using normal brain tissue from a commercial source (NOVUS; NBP2-77523), harvested from a 12 years-old male without signs of disease or neuropathology. Comparisons were performed with matching images collected from regions-of-interest (ROI) at equivalent depths from the cortex surface (measured in mm), because use of layering structure as a proxy for localization inside the tissue was not possible for PTHS brain tissue, due to its characteristic disorganized anatomy. No significant difference was observed by the pathologists in terms of general appearance of the brain gyri and width of the cortex tissue prior to dissection.

Organoid single cell RNA sequencing analysis. CtOs and sPOs were dissociated to produce a single cell suspension via a combination of mechanical dissociation with forceps and enzymatic digestion with Accutase for 10 min. For each library, a total of 15 organoids were dissociated and the resulting cells were pooled and subsequently filtered to isolate single cells for RNA sequencing analysis on the same day. Dissociated cells were pelleted (3 min, 100× g, 4° C.) and resuspended in 10 mL of Neurobasal medium. The concentration of single cells in each library was determined using the Chemometec automatic cell counter, and the minimum population viability across all libraries was found to be 85%. Single cell RNA-seq libraries were prepared using the Chromium Single Cell 3′ v3 Library kit (10× Genomics) according to the manufacturer's protocol. Approximately 20,000 cells were loaded per sample on the Chromium chip. All steps, including GEM (Gel beads in emulsion) preparation, reverse transcription, PCR amplification, and Illumina library construction were carried out on a T100 thermal cycler (Bio-Rad). cDNA extracted from GEMs was cleaned up using MyOne Silane Beads (Thermo Fisher Scientific), PCR-amplified for a total of 10 cycles, and then purified using SPRIselect Reagent Kit (B23317, Beckman Coulter). Next, the cDNA pool was enzymatically fragmented for each library and a double size selection was performed using the SPRIselect Reagent Kit. Finally, Illumina adapters were ligated to prepare libraries for sequencing, followed by another round of double size selection with the SPRIselect Reagent Kit. Final library sizes ranged from 300-700 bp, with an average size around 450 bp. Illumina libraries were quantified using Qubit dsDNA HS Assay Kit (Thermo Fisher Scientific) and size quality control was performed on a High Sensitivity D1000 tapestation (Agilent). Libraries were sequenced on a NovaSeq 6000 S4 sequencer (Illumina) to produce 20,000 reads per single cell, or 400 million reads per library, with 26 cycles for read_1, 8 cycles for the index, and 98 cycles for read_2, which contains the gene sequence.

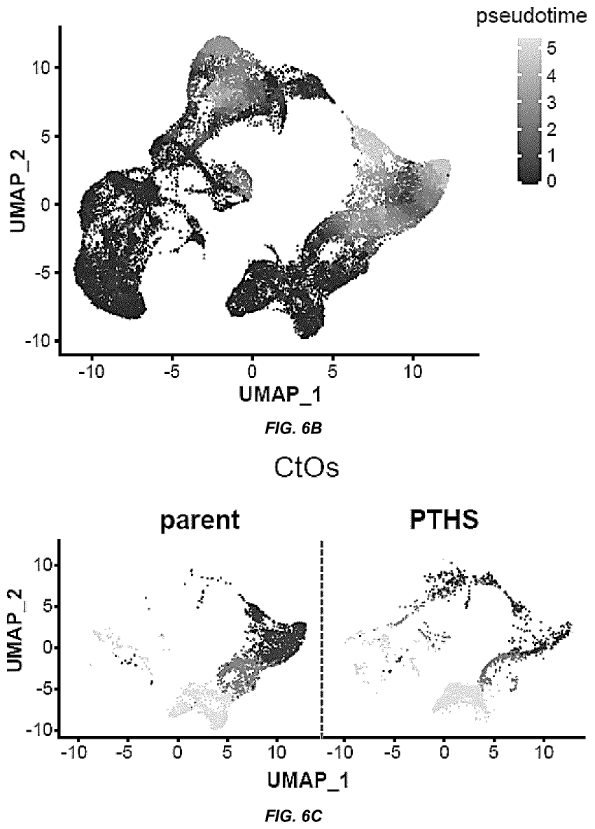

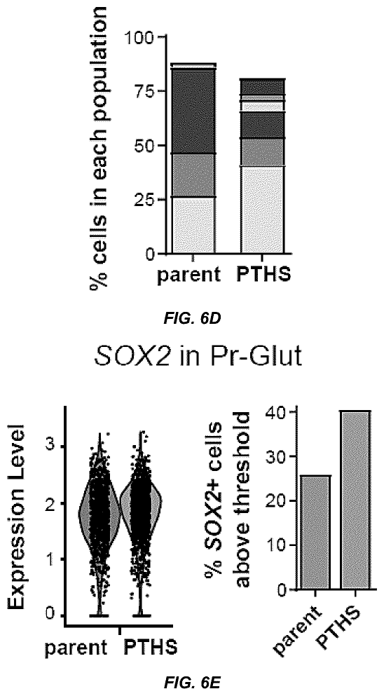

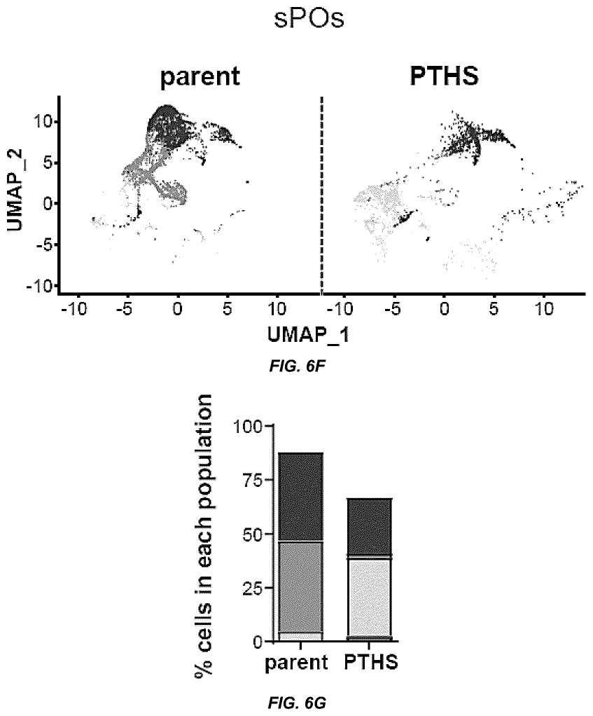

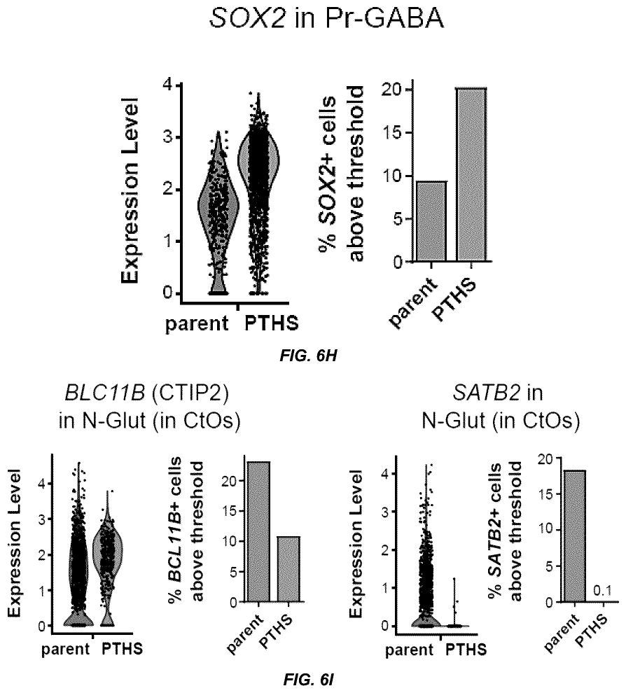

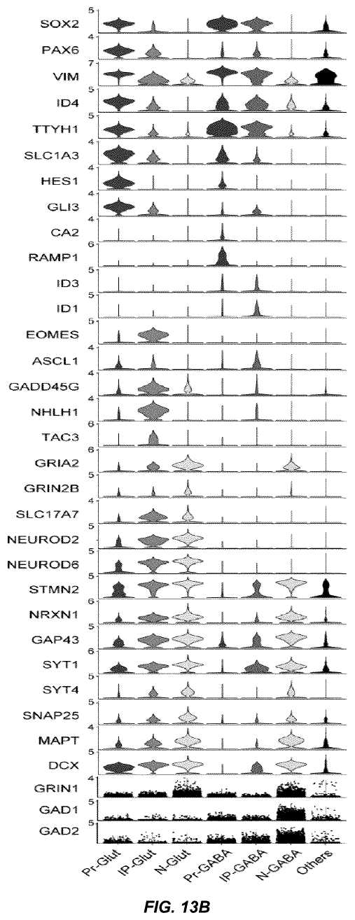

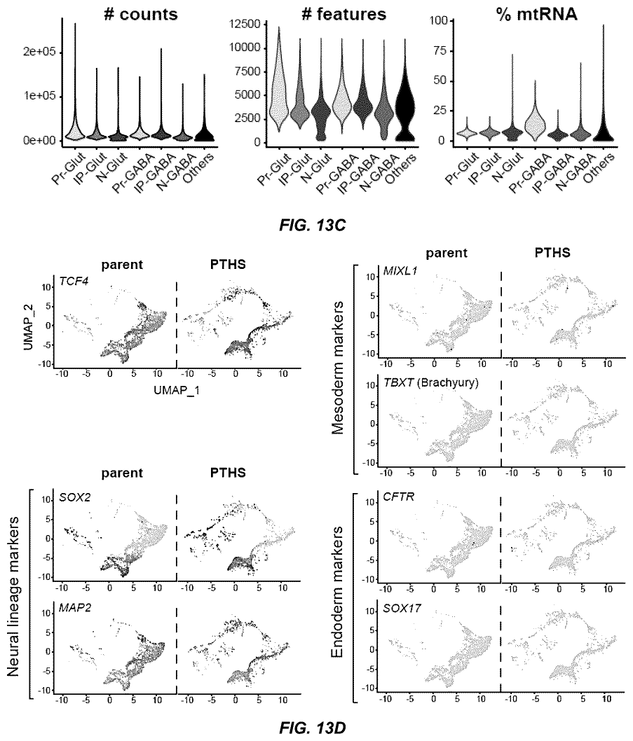

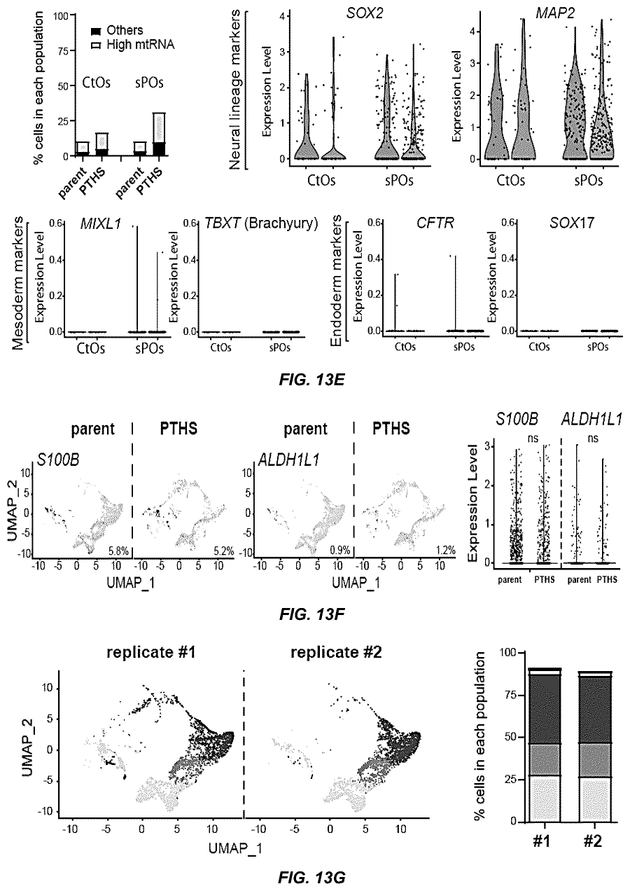

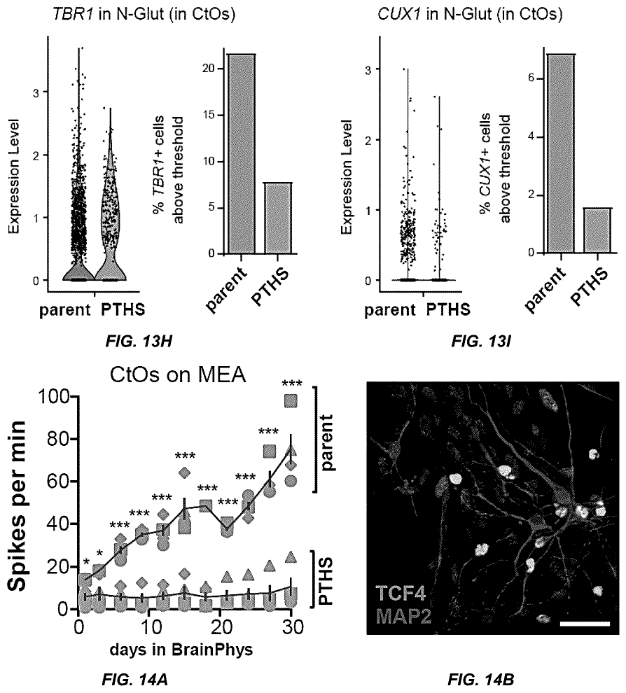

Feature count matrices for each single-cell RNA-Seq library were generated separately using the ‘cellranger count’ command in the Cell Ranger (version 4.0.0) software and the GRCh38 2020-A reference dataset of human transcripts. The independent libraries were then normalized to the same sequencing depth and aggregated into a single feature-barcode matrix using the ‘cellranger aggr’ command. Cell type subpopulations were delineated by a combination of automated annotation and curated manual inspection. First, processed data were transferred to Cell Loupe software (10× Genomics) and analyzed to partition groups of single cells using k-means clustering with 8 groups. The expression of marker genes was then visually inspected in each subpopulation assigned by Cell Loupe. Next, the expression of each marker gene was analyzed in FIG. 13B and manually adjusted to the k-mean-assigned subpopulations based on the expression pattern of these genes. The combined approach of first performing unbiased determination of subpopulations followed by manual curation maximizes the identification of biologically relevant groups of cells. It is evident that these subpopulations could be further subdivided into other groups of cells, but it was decided to focus on groups containing progenitors, intermediate progenitors, and neurons in the excitatory and inhibitory lineages shown by the single cell data (FIG. 6A-J). Other minor subpopulations exist but were not displayed in most panels because the focus was on the 6 subpopulations mentioned above. The other populations were collectively named ‘Others’ in FIGS. 13A-B, and further analysis led to the conclusion that the number of cells in this category does not differ between parent and PTHS, and they are not cells of non-neural origin, which are not present in conspicuous amounts in CtOs or sPOs (FIG. 13E). Mitochondrial genes were used as a proxy for identifying apoptotic cells, which were generally infrequent in all libraries (less than 5%).

Next, the Seurat library (version 3.2.2) (Butler et al., 2018) was used for downstream processing and analysis of the feature-barcode matrix. First, the aggregated matrix generated by Cell Ranger was imported into Seurat and normalized by dividing the feature counts of each cell by the total counts for that cell, followed by scaling the data to 10,000 counts per cell prior to performing a log transformation (‘NormalizeData’ function). Next, variable features were identified with the ‘FindVariableFunctions’ function, which fits a polynomial curve to the mean-variance relationship, standardizes feature counts based on their expected variance given their expression, and selects the 3,000 features with the highest variance. The highly variable features were then scaled to a distribution (‘ScaleData’ function) with mean expression 0 and variance 1 across cells, which was subsequently used to perform linear dimensional reduction (PCA, ‘RunPCA’ function). The first 15 PCA dimensions were used to embed the cells into a non-linear reduced dimension space using the UMAP algorithm (‘RunUMAP’ function).

Unsupervised trajectory (pseudotime) inference (FIG. 6B) was performed independently for the excitatory and inhibitory lineages using Monocle 3 (version 0.2.2) (Cao et al., 2019). Specifically, the Leiden method was used to cluster cells within the UMAP embedding (‘cluster_cells’ function), and unpartitioned principal graphs representing the differentiation trajectories were then fit to the data (‘learn_graph’ function). Finally, cells were ordered by rooting the trajectories at the manually annotated progenitor subpopulations (‘order_cells’ function). Pseudotime is the transcriptional distance (abstract units) between a cell and the start of the trajectory, measured along the shortest path.

Cell Loupe software was used to quantify the percentages of cells in each subpopulation and library (FIGS. 6D, 6G, 13F, 13G, 16H and 17K). To calculate the percentage of cells expressing a certain gene, Seurat and R package were used to count the number of cells expressing said gene above a threshold level corresponding to 40% of the gene expression mean in each group being compared (FIGS. 6E, 6H-J, 7F, 13H, 13I and 17L). For the statistical comparison of gene expression levels between specified subpopulations, Mann-Whitney U test (for 2 groups) or Kruskal-Wallis test were used followed by Dunn's post-hoc test (for more than 2 groups and pairwise comparisons).

NPC derivation and neuronal differentiation. iPSC colonies maintained in mTeSR1 medium were switched to DMEM/F12 medium containing N2 and GEM21 supplements (StemCell Technologies). After 2 days, colonies were lifted from the plate with Accutase and cultured in the same medium with the addition of 10 mM SB431542 and 1 mM dorsomorphin, in suspension on a platform shaker, until embryoid bodies formed. After 2 weeks of culturing in this manner, the embryoid bodies were plated directly onto Matrigel-coated dishes and maintained in DMEM/F12 medium containing N2 and SM1 supplements (StemCell Technologies), 20 ng/mL FGF-2, and 1% penicillin/streptomycin. After 3 to 5 days, rosettes emerged, and 7 days later the rosettes were manually picked and replated onto Matrigel-coated dishes. NPCs sprouted around the rosettes and were dissociated with Accutase for 5 min prior to being reseeded onto plates coated with 10 μg/mL poly-ornithine (Sigma-Aldrich) and 5 μg/mL laminin (Thermo Fisher Scientific) to produce passage 1 (P1). NPCs were maintained in DMEM/F12 medium containing N2 and SM1 supplements, 20 ng/mL FGF-2, and 1% penicillin/streptomycin for up to 20 passages. The cultures were not derive in medium containing Wnt or Shh agonists/antagonists, such as cyclopamine, because treatment of progenitors with artificially high concentrations of these substances might affect the cells' proliferation rate, thereby potentially adding a confound factor in the evaluations of NPC proliferation.

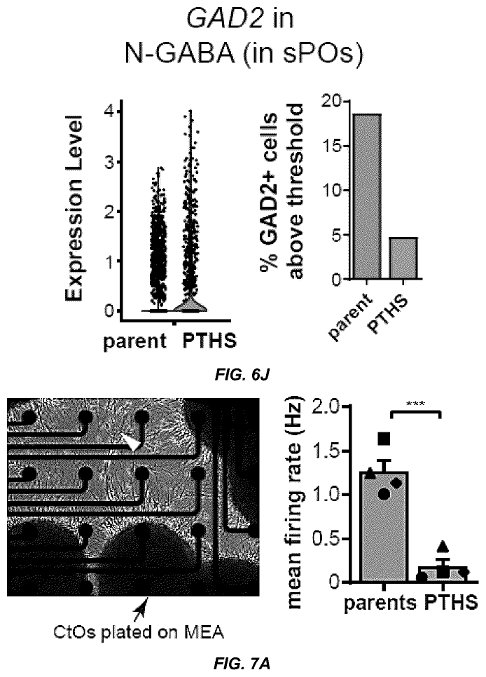

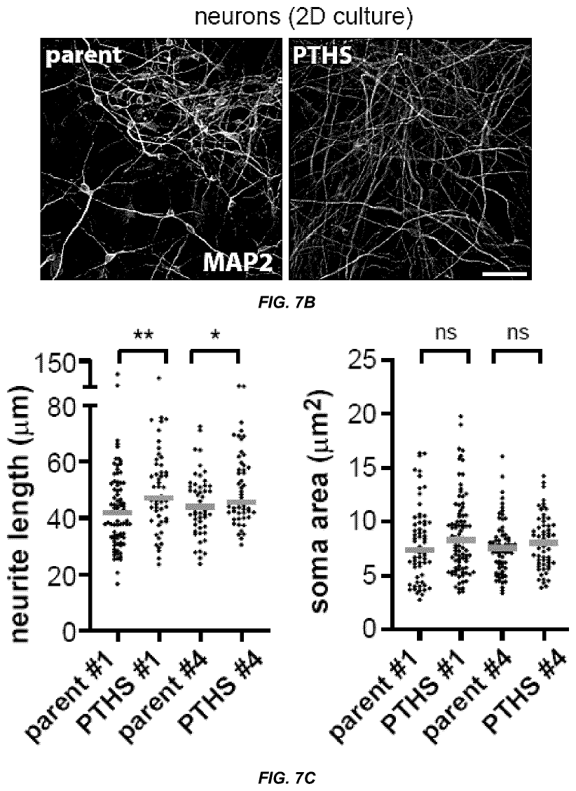

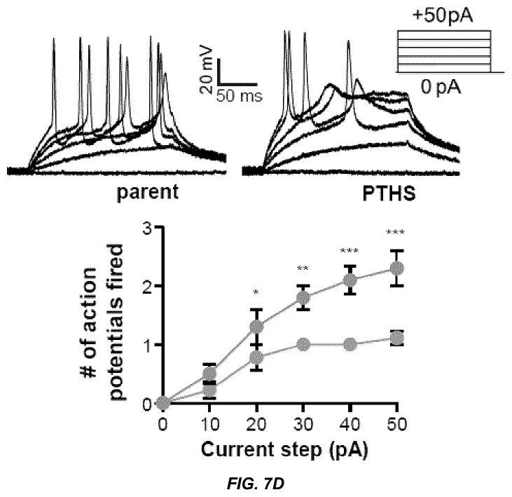

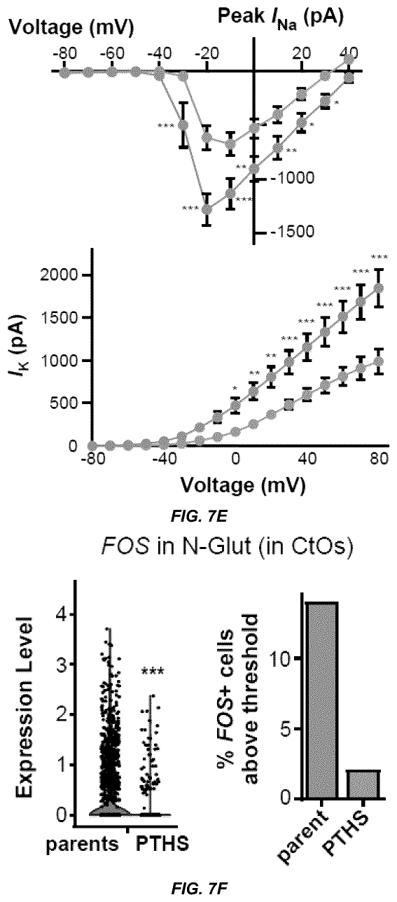

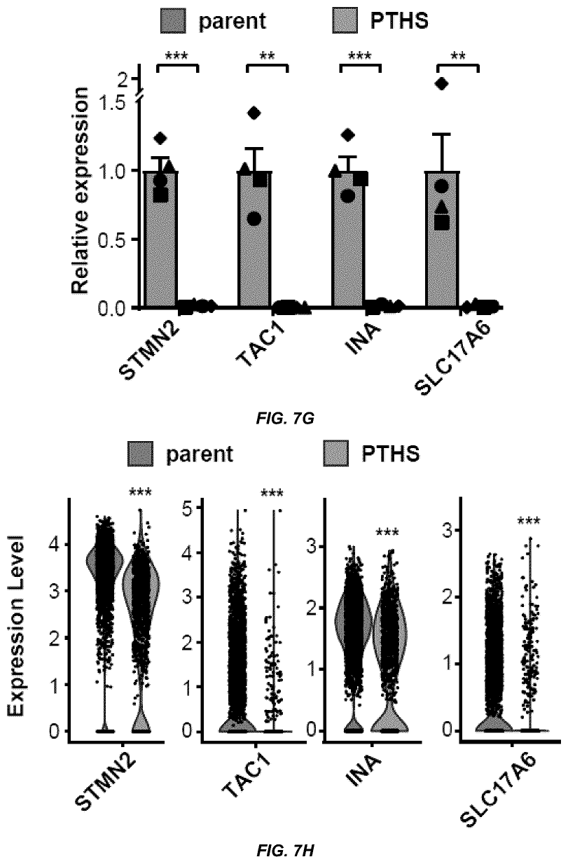

For neuronal differentiation, NPCs were seeded onto plates coated with poly-ornithine and laminin and cultured in NPC medium until they reached 90% confluency, at which time the medium was changed to DMEM/F12 containing N2 and SM1 supplements and 1-penicillin/streptomycin, with media changes occurring every 3 to 4 days. When neuronal processes started to grow one week later, the medium was changed to BrainPhys neuronal medium (StemCell Technologies) and the cells remained under these conditions for up to 4 months, with media changes occurring every 3 to 4 days. Electrophysiological measurements in FIGS. 7D-E were performed on neuronal cultures after 3 or 4 months in BrainPhys medium, a time point at which the vast majority of cells in the culture are MAP2+(95.4±2.4% in parent versus 93.2±1.4?. in PTHS; P=2.4; unpaired Welch's t-test).

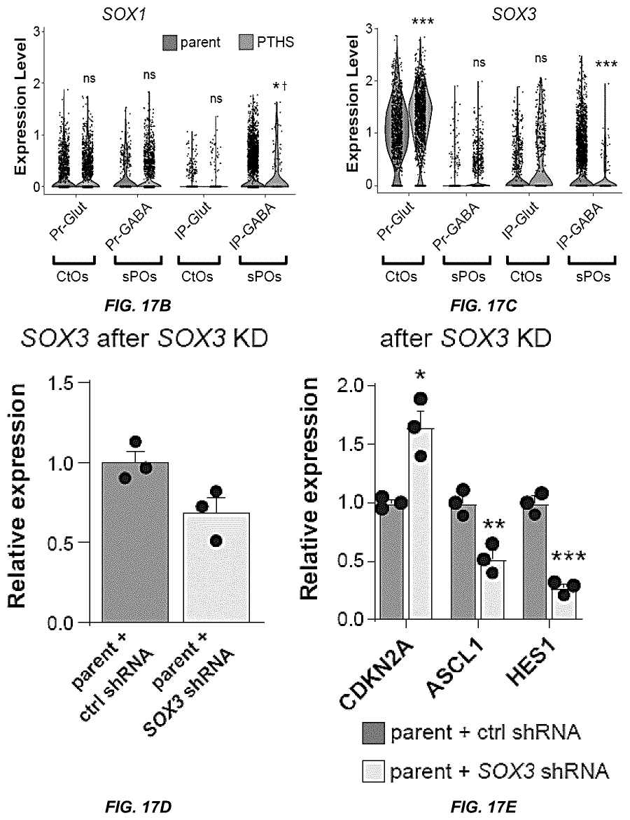

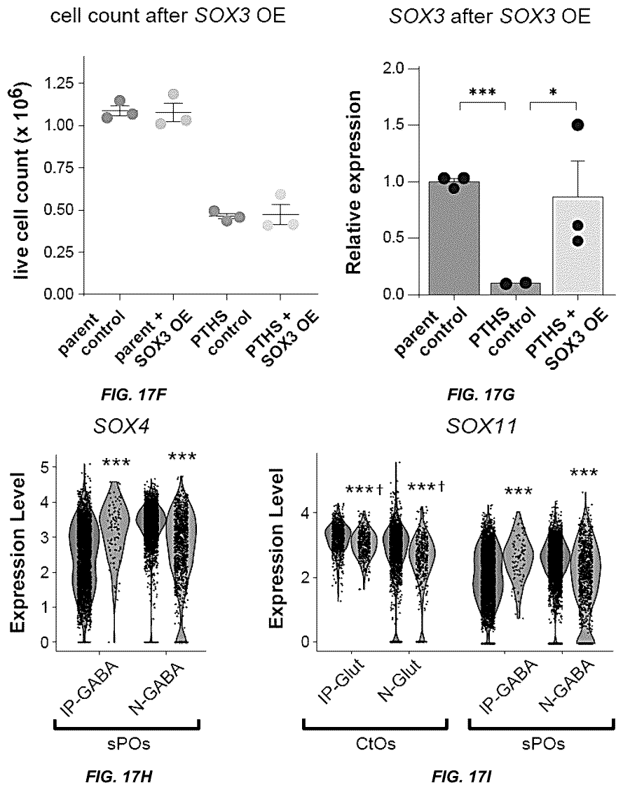

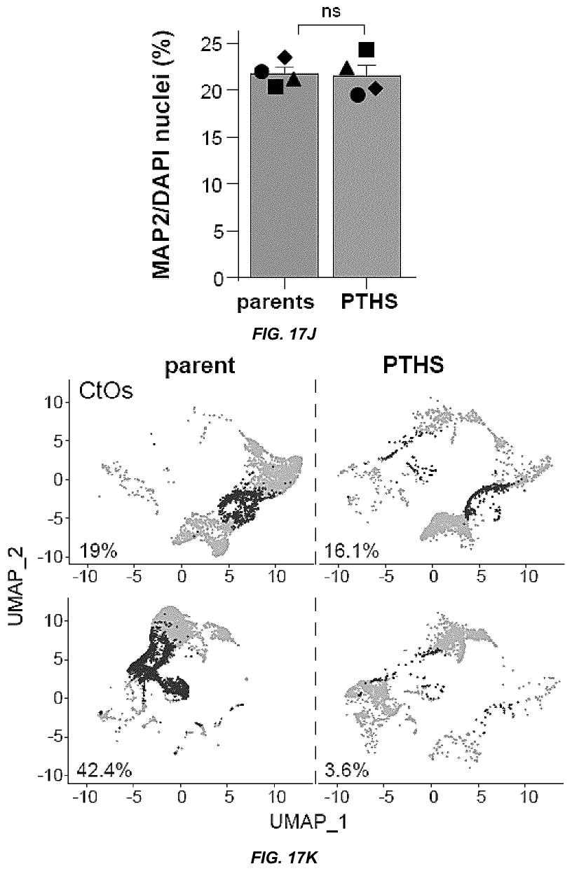

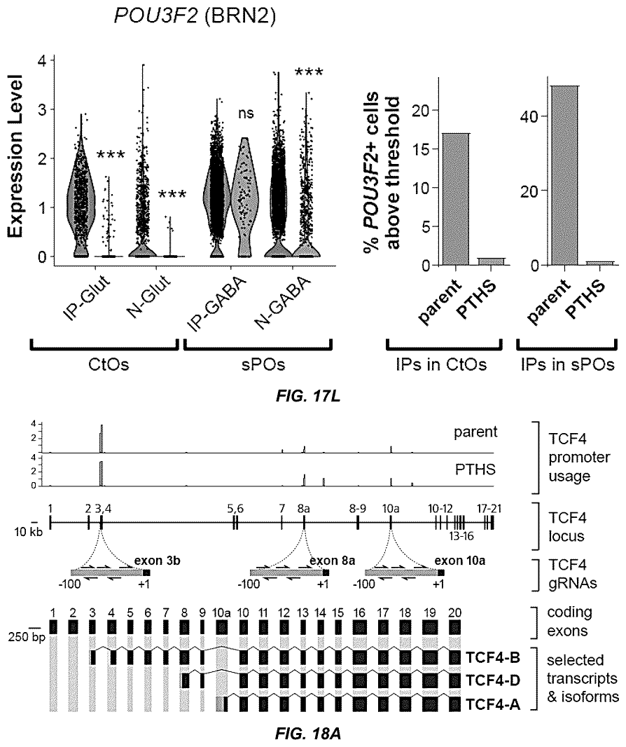

Quantification of neuronal differentiation rates (FIGS. 10J, 10K and 17J) was accomplished by counting MAP2+ and SOX2+ cells in differentiating neuronal cultures seeded onto LabTek II chambered slides after 2 months of differentiation in BrainPhys medium, followed by immiunofluorescence staining as described before.

RNA sequencing of NPCs and neuronal cultures. Using the RNeasy Mini Plus kit (Qiagen), RNA was isolated from NPCs of 4 subjects and 4 respective parental controls at passage 15 for most analyses, from NPCs of 2 subjects and 2 respective controls at passage 5 for analysis in FIG. 15H and from differentiating neuronal cultures after 2 months in BrainPhys medium (one patient and respective parental control) subjected to FACS sorting to purify the CD184+/CD44−/CD24+ population (FIGS. 7G, 14C, 14F, 14G and 18L). For each subject, RNA was extracted from 3 independently prepared biological replicates. A total of 1 μg of RNA was used from each sample for Illumina library preparation using the stranded TruSeq kit (Illumina). RNAs were sequenced on Illumina NovaSeq 6000 S4 instrument with 150 bp paired-end reads, generating approximately 40 million sequencing fragments per library.

To estimate transcript-level expression from bulk RNA-Seq data, Salmon (version 0.14.1) software (Patro et al., 2017) was used, with selective mapping (‘-validateMappings’) and correction for sequence-specific biases (‘-seqBias’), GC-content biases (‘-seqBias’), and fragment position bias (‘-posBias’). Reference transcripts for read mapping were obtained from the GENCODE 32 basic annotation (Frankish et al., 2019). For every sample, outliers were defined by high between-replicate Euclidean distances (after transformation to achieve homoskedasticity, as described elsewhere herein), which led to the exclusion of just one library replicate from PTHS patient #3 from the follow-up expression analysis. All remaining 41 libraries passed the quality control phase and were retained.

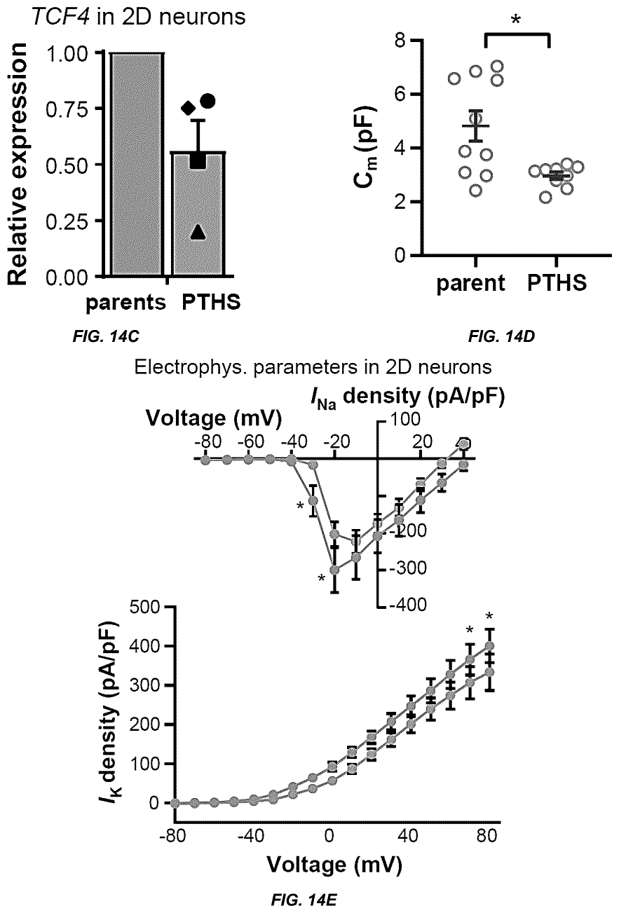

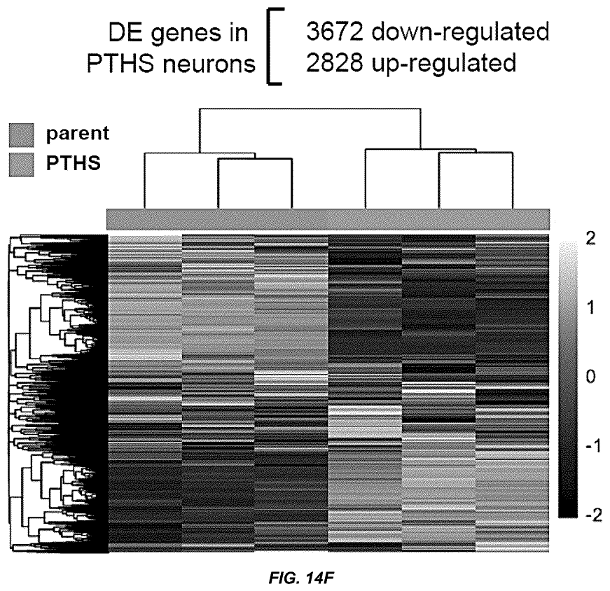

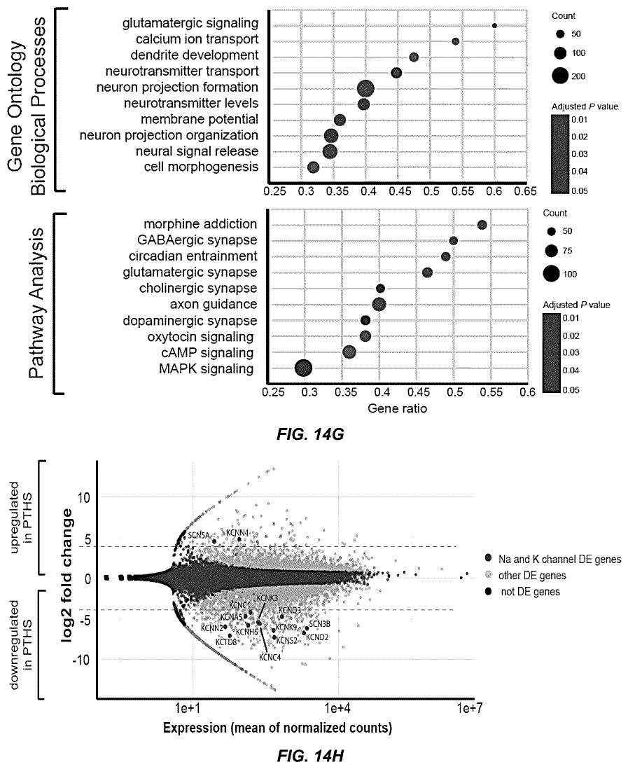

Pairwise differential expression (DE) tests between cells derived from PTHS patients and their respective parental controls were performed with DESeq2 (version 1.22.1) (Love et al., 2014). tximport (version 1.10.1) (Soneson et al., 2015) was employed to aggregate transcript abundances into gene-level counts. Next, between-sample normalization was performed using the size factors approach (Anders and Huber, 2010) and a local dispersion model was fit to the normalized counts. Lastly, a negative binomial generalized linear model was fit to the data, the effect sizes (log 2FoldChange) were shrunken with the apeglm algorithm (Zhu et al., 2019), and strict statistical testing was accomplished using threshold-based Wald tests (‘lfcThreshold=0.5’). DE transcripts were determined based on their s-values (<0.005). Transformation of count data into an approximately homoskedastic matrix for clustering and visualization purposes (FIGS. 14F and 15M) was attained with the ‘varianceStabilizingTransformation’ function using the ‘blind’ parameter set to ‘TRUE’.

To obtain lists of DE genes across all subjects, a list of DE genes between each PTHS subject and his respective parent (Table 3) was first derived, followed by the cross examination between the 4 lists and selection of DE genes common to all 4 child-parent pairs. The final list was then used for gene-set enrichment assessment followed by Gene Ontology (GO) and pathway analyses, using the web-based WebGestalt tool (Wang et al., 2017), with default parameters. WebGestalt conducts permutations to obtain an over-representation Z score and enrichment p-value for each GO term. For pathway analysis, the KEGG option, with default parameters was used. For all analyses, a minimum of 5 genes per category was employed, with BH multiple test correction, and a significance level chosen for a false-discovery rate of 0.05.

Real time quantitative PCR. Total RNA was extracted using RNeasy Mini Plus kit (Qiagen), followed by DNase I treatment on the column, as per the manufacturer's recommendations. Two and a half micrograms of total RNA were reverse transcribed into cDNA using the Superscript III First-Strand Reverse Transcription System (Thermo Fisher Scientific). Real-time quantitative PCR (RT-qPCR) was performed using pre-validated FAM-MGB TaqMan probes (Thermo Fisher Scientific) and the TaqMan universal master mix II without UNG (Thermo Fisher Scientific) on a CFX Connect Real Time PCR detection system (Bio-Rad), with the following cycling parameters: 94° C. for 3 min, followed by 40 cycles of 94° C. for 30 s and 68° C. for 1 min. Amplification and denaturation curves for all probes were analyzed to verify amplification of just one amplicon. All RT-qPCR analyses were conducted using RNA extracted from at least 3 independent biological samples per subject/condition, and normalized to the following endogenous control genes (TBP, ACTB, and GAPDH). Relative expression was calculated using the traditional ΔΔCt method.

The following TaqMan probes were used: STMN2 (Hs00199796_m1), TAC1 (Hs00243225_m1), INA (Hs00190771_m1), SLC17A6 (Hs00220439 m1), CDKN2A (Hs00923894 m1), LMNB1 (Hs01059210 m1), WNT2B (Hs00921615_m1), WNT3 (Hs00902257_m1), WNT5A (Hs00180103_m1), SFRP2 (Hs00293258_m1), ASCL1 (Hs00269932_m1), NEUROD1 (Hs00159598_m1), HES1 (Hs00172878_m1), SOX2 (Hs04234836_s1), SOX3 (Hs00271627_s1), SOX4 (Hs00268388_s1), TCF4 (Hs00972432_m1), CNTNAP2 (Hs01034283 m1), GADD45G (Hs00198672 m1), MAP2 (Hs01103234_g1), VIM (Hs00185584_m1), NES (Hs04187831_g1), ID3 (Hs00171409_m1), KCNQ1 (Hs00165003_m1), BCL11B (CTIP2) (Hs00256257_m1), SATB2 (Hs00392652_m1), CUX1 (Hs00738851_m1), TBR1 (Hs00232429_m1), CDH23 (Hs00254446_m1), and PCDH15 (Hs00263709_m1).

Neuronal morphometric measurements. Neurons were morphologically analyzed (FIG. 7C) using Neurolucida Neuron Tracing Software (MBF Bioscience). Individual MAP2+ neurons were identified from confocal images that clearly exhibited either the number of processes branching from the cell body, processes of complete root-to-tip length, or complete cell bodies. Only neurons whose shortest dendrite was at least 3 times longer than the diameter of the cell soma were calculated. Random images from at least 2 clones of each cell line were assessed. The ‘contour’ function was used to trace and sum incremental lengths of each curve along the longest path of a complete process to yield its total length. The outlines of cell bodies were also traced using the ‘contour’ function and the resulting surface areas were automatically calculated by the software.

Multi-electrode array analysis. 12-well multi-electrode array plates from Axion Biosystems were used to acquire electrical activity reads from organoids. Six organoids were plated onto each well at 20 days into the organoid derivation protocol described herein, using Neurobasal medium containing GlutaMAX, 1% Gem21, 1% NEAA, 10 ng/mL of BDNF, 10 ng/mL of GDNF, 10 ng/mL of NT-3, 200 mM L-ascorbic acid and 1 mM dibutyryl-cAMP. They were maintained in this medium for 7 days, then switched to Neurobasal medium containing 1% GlutaMAX, 1% Gem21, 1% NEAA, and 0.5% penicillin/streptomycin for an additional 7 days. After this timeframe, the seeded organoids were kept in BrainPhys medium until the time of measurement. At least 2 independent experiments were conducted for each subject, with 3 independent replicates per subject in each experiment. Organoids were assessed for electrophysiological parameters starting 7 days after switching to BrainPhys medium. Data reported in FIG. 7A were from organoids cultured for 30 days in BrainPhys medium. Data reported in FIG. 11G were from organoids cultured for up to 90 days in BrainPhys medium.

Recordings were performed using a Maestro system and AxIS software (Axion Biosystems), with a bandwidth filter from 10 Hz to 2.5 kHz. Spike detection was computed with an adaptive threshold of 5.5 times the standard deviation of the estimated noise for each electrode. Plates were left untouched in the Maestro instrument for 3 min prior to recording, which proceeded for 3 additional minutes. Data was analyzed using the Axion Biosystems Neural Metrics Tool, under the condition that an electrode was deemed active if it had at least 5 spikes occur over 1 minute (5 spikes/min minimum). The mean firing rate for a subject was calculated across active electrodes in all wells for that subject. Network bursts were defined as bursts of more than 10 spikes that occurred in more than 25G of the active electrodes in the well, with a maximum inter-spike interval of 100 ms.

Patch clamp electrophysiological analysis. Whole-cell patch clamp recordings were performed on neurons in bidimensional (monolayer) culture, differentiated from NPCs on 35 mm dishes coated with poly-ornithine and laminin for 4 months after withdrawal of FGF-2. Similar densities of neurons were achieved in all plates. The extracellular solution was 130 mM NaCl, 3 mM KCl, 1 mM CaCl₂, 1 mM MgCl₂, 10 mM HEPES, and 10 mM glucose, at pH 7.4 adjusted with 1 M NaOH (˜4 mM Na⁺ added). The internal solution for glass electrodes was 138 mM K-gluconate, 4 mM KCl, 10 mM Na₂-phosphocreatine, 0.2 mM CaCl₂, 10 mM HEPES (Na⁺ salt), 1 mM EGTA, 4 mM Mg-ATP, 0.3 mM Na-GTP, at pH 7.4 adjusted with 1 M KOH (˜3 mM K⁺ added). The osmolarity of all solutions was adjusted to 290 mOsm. Filamented borosilicate glass capillaries (1.2 mm OD, 0.69 mm ID, World Precision Instruments) were pulled on a Flaming/Brown micropipette puller (Model P-87, Sutter Instrument). The electrode resistances were 4-6 M0 for the whole-cell recording. Axon CV-4 headstage and Axopatch 200A amplifier (Molecular Devices) were used for the electrophysiological recordings at room temperature. For evoked AP recordings, current-clamp configuration was employed with the injection of small currents to maintain the membrane potential at −70 mV. Then, voltage-clamp configuration was used to record voltage-dependent neuronal Na⁺ and K⁺ currents. Recordings were low-pass filtered at 1 kHz, and digitized at 10 kHz using a DigiData 1322A (Molecular Devices). Liquid junction potentials were nulled. Electrophysiology data were analyzed offline using pCLAMP 10 software (Molecular Devices). Statistical comparisons were performed with 10 (parent) or 9 (PTHS) neurons per group, using two-tailed Welch's t test with a significance threshold of p=0.05.

Proliferation and apoptosis assays. For quantifying cell proliferation via cell counting, individual wells of a 12-well poly-ornithine/laminin-coated plate were seeded with 100,000 cells per well. At least 2 experiments were conducted per subject, with three technical replicates per subject per experiment. After the indicated number of days, cells were lifted by Accutase treatment for 5 min, resuspended in equal volumes of DMEM/F12 and counted using a Chemometec Via-1 cassette, which also calculated total live cell counts.

For EdU cell cycle assays, Click-iT EdU Flow Cytometry Assay Kit (Thermo Fisher Scientific) was used, following the manufacturer's protocol. Briefly, 70% confluent NPCs from 10 cm dishes were dissociated with Accutase, resuspended in StemDiff Neural Progenitor Medium (StemCell Technologies) and plated onto Matrigel-coated 6-well plates at a density of 0.2×10⁶ cells/well. Cells were incubated at 37° C. and 5% CO₂ for 12 h before EdU was added to the culture medium at a final concentration of 10 μM. Cells were incubated for another 2.5 h for EdU incorporation, and subsequently harvested by Accutase-mediated dissociation, resuspension in 3 mL of 1% BSA in 1×PBS, and pelleting at 500×g for 5 min. Pellets were resuspended and incubated in the kit's fixative solution for 15 min in the dark at 25° C., followed by the addition of 3 mL of 1% BSA in 1×PBS to stop fixation. Next, NPCs were pelleted at 500×g for 5 min, the supernatant was removed, and the pelleted cells were incubated for 15 min in 1× Click-iT saponin-based permeabilization and wash reagent. During incubation, the Click-iT reaction cocktail was prepared based on the manufacturer's protocol, and then added to the samples, followed by homogenization and incubation for 30 min, protected from light. Cells were re-homogenized every 5 min and then washed in 3 mL of 1× Click-iT permeabilization and wash reagent, pelleted, and resuspended in the same solution, before nuclear staining in 1×PBS/0.1% Triton X-100/100 μg/mL RNase A solution containing 20 μg/mL propidium iodide. Immediately after nuclear staining, cells were transferred to ice and kept at 4° C., protected from light, until analysis in an LSR Fortessa X-20 cell cytometer (BD Biosciences).

Apoptosis assays on NPCs were conducted using the Dead Cell Apoptosis Kit with Annexin V FITC and PI (V13242, Thermo Fisher Scientific), following the manufacturer's protocol, and analysis by flow cytometry in the same instrument described above.

TOP-Flash luciferase reporter Wnt functional assay. For assessing levels of Wnt signaling, 70% confluent cultures of NPCs in 24-well plates (with Neural Progenitor Medium from StemCell Technologies) were transfected with the M50 Super 8×TOPFlash plasmid (Addgene #12456; [http:/]/n2t.net/addgene:12456; RRID:Addgene_12456; referred to as TOP-Flash luciferase reporter plasmid), which is used to assess β-catenin-mediated transcriptional activation. This plasmid contains a minimal TA viral promoter driving the expression of a firefly luciferase gene preceded by seven binding sites (AGATCAAAGG; SEQ ID NO:1) for TCF/LEF (Veeman et al., 2003), not to be confused with TCF4. Control NPCs were transfected with M51 Super8×TOPflash plasmids, which have mutant TCF/LEF binding sites (Addgene plasmid #12457; [http:/]/n2t.net/addgene:12457; RRID:Addgene_12457).

Transfection was performed using the Amaxa Nucleofaction Mouse Neural Stem Cell Nucleofector kit for NPCs (Lonza), using the manufacturer's recommendations. After 24 h, medium was replenished and the luciferase assay was performed using the Pierce Firefly Luciferase Flash Assay Kit (Thermo Fisher Scientific) on a sample of 50,000 cells, using a Synergy microplate reader (BioTek Instruments). All assays were conducted on 3 independent replicates per NPC line (per subject) and 3 technical replicates. Activity levels were expressed as arbitrary units normalized against the mean activity in the respective controls.

Wnt signaling manipulation. For manipulating the Wnt/β-catenin signaling pathway in NPCs, 200,000 cells were seeded on a 6-well plate, followed by treatment with specific agonist CHIR99021 (1 μM) for 4 days. Controls were treated with DMSO (CHIR diluent) at the same concentration and for the same duration. In separate experiments, cells were treated with Wnt signaling antagonists DKK-1 (25 μM) or ICG-001 (1 μM) for 3 to 5 days. In all cases, treated cells were assayed to measure activity of the Wnt pathway via transfection with the TOP-Flash plasmids described herein. For all experiments, 3 biological replicates were used per subject line, and similar results were obtained in at least 3 independent experiments.

CtOs or sPOs were treated with 1 μM CHIR99021 (or DMSO, as a control) on the first day of the progenitor proliferation phase (when FGF-2 is first added to the growing organoids), in the same type of medium as untreated organoids. Similarly, treatment with Wnt antagonist ICG-001 (1 μM) was performed on the first day of the progenitor proliferation phase. In all cases, treatment was performed on at least 6 independent replicates of each organoid line.

TCF4, SOX3 and SOX4 knockdown. For TCF4 and SOX3 knockdown in NPCs, 100,000 cells were tranfected using the Amaxa Nucleofaction Mouse Neural Stem Cell Nucleofector kit (Lonza) with shRNA Mission plasmids (Sigma Millipore), using the manufacturer's recommendations. The SHCLND-NM-005834 (SOX3) and SHCLND-NM_003199 (TCF4) pre-validated Mission shRNA vectors (Sigma Millipore), which are made in the pLKO.1 plasmid backbone (TRC2 series) were used. SHC201 empty TRC2 vector was used as a control. Four days after transfection, cells were counted and RNA was extracted using the RNeasy Mini Plus kit (Qiagen), followed by analysis of gene expression for selected genes via RT-qPCR. Each experiment was conducted with 3 replicates per subject/condition for cell counting or 3 independent replicates for RNA extraction and gene expression assessment. Because no selection was applied after transfection, the observed effects of SOX3 or TCF4 knockdown on the expression of other genes should be interpreted as the mean variation across all cells in the transfected population. This may explain why, for example, TCF4 knockdown in NPCs leads to reduction in SOX3 expression (FIG. 10C) with an effect size smaller than the one observed in the comparison between control and PTHS NPC samples (FIG. 10B).

For SOX4 knockdown in neurons (FIG. 8I-J), methods that would require transfection of the differentiating neuronal culture were avoided, as this would result in phenotypic alterations, cell death, and changes in cell density. Therefore, an antisense oligonucleotides (ASOs) approach was used, two of which were used in combination in all experiments. ASOs were designed using the manufacturer's design tool as antisense 16 nucleotides long locked nucleic acid (LNA) oligos (Qiagen), which are enriched with LNAs in the flanking regions but contain regular DNA nucleotides in a LNA-free central gap (GapmeRs). Each ASO was resuspended in 10 mM Tris pH 7.5/0.1 mM EDTA and used at 1 μM final concentration. Two weeks after withdrawal of FGF-2, differentiating neuronal cultures were treated with ASOs on days 15, 20 and 25 after withdrawal of FGF-2 via direct application to the culture medium for unassisted uptake (gymnosis). Cultures were fixed or harvested for RNA extraction three days after the last treatment with ASOs.

SOX3 overexpression. For SOX3 overexpression in NPCs (FIG. 17F-G), 100,000 cells were transfected using the Amaxa Nucleofaction Mouse Neural Stem Cell Nucleofector kit (Lonza) with the 1.5 μg of pENTER-CMV-SOX3 plasmid (Vigene Biosciences; CH850241), in which the SOX3 coding sequence is controlled by the cytomegalovirus (CMV) promoter. Four days after transfection, cells were counted and RNA was extracted using the RNeasy Mini Plus kit (Qiagen), followed by analysis of gene expression for selected genes via RT-qPCR, as described above. Each experiment was conducted with 3 replicates per subject/condition for cell counting or 3 independent replicates for RNA extraction.

TCF4 overexpression. Prior to testing CRIPSR-mediated enhancement of TCF4 expression via trans-epigenetic activation of the endogenous locus, the effects of overexpressing TCF4 were tested by transfecting control and PTHS NPCs with cassettes in which the TCF4-B transcript variant coding sequence was placed under the control of artificial promoters (FIGS. 11E and 18J). For control conditions, the coding sequence was placed under control of the artificial minP promoter (AGAGGGTATATAATGGAAGCTCGACTTCCAG; SEQ ID NO:2). Other constructs contained the TCF4-B coding sequence preceded by the minP promoter and by varying numbers (6 or 12) of pE5 TCF4 regulatory DNA binding sites (CACCTG) separated by spacer sequences composed of CAAGAA. These constructs were prepared via PCR-based reactions to ligate Ultramer oligonucleotides (minP_TCF4, E-box-x6-minP TCF4, or E-box-x12-minP TCF4; Integrated DNA technologies) containing the artificial promoter to the TCF4-B coding sequence, which was separately amplified via RT-PCR from human brain cDNA (Promega) using primers TCF4B_cDNA Forward and TCF4B_cDNA Reverse. The resulting PCR fragments were cloned into EcoRI and XhoI restriction sites of pLenti-III-promoterless vector (Applied Biological Materials). NPCs were transfected with these plasmids using the protocol as described herein, followed by extraction of total RNA with the RNeasy Mini Plus kit (Qiagen) and RT-qPCR, as before.

TABLE 2
Reagents

| Oligonucleotides | SEQ ID NO: |
|---|---|
| gRNA sequence: 3bS1: | 11 |
| CTTTATAAGCCCGCAGTTCC | |
| gRNA sequence: 3bS2: | 12 |
| CCGCAGTTCCCGGATGTGAA | |
| gRNA sequence: 3bS3: | 13 |
| GTCGACCAGCACCGCCATCT | |
| gRNA sequence: 3bA1: | 14 |
| GGTAAACAGAGCGCCTAGAG | |
| gRNA sequence: 3bA2: | 15 |
| CATTCACATCCGGGAACTGC | |
| gRNA sequence: 8aS1: | 16 |
| ACATAGGAAGGTACGACTTC | |
| gRNA sequence: 8aS2: | 17 |
| TTTACGTACCAGACATAGGA | |
| gRNA sequence: 8aS3: | 18 |
| TTCCTTTACGTACCAGACAT | |
| gRNA sequence: 8aA1: | 19 |
| TTCCTATGTCTGGTACGTAA | |
| gRNA sequence: 8aA2: | 20 |
| AAGTCGTACCTTCCTATGTC | |
| gRNA sequence: 10aS1: | 21 |
| CACGCTTGGCCCGGCCATAT | |
| gRNA sequence: 10aS2: | 22 |
| CTTTGCATATTCACCACGCT | |
| gRNA sequence: 10aS3: | 23 |
| AGCGTCTGACAGCAGCGCCG | |
| gRNA sequence: 10aA1: | 24 |
| TCAACTTTGCGCAGCGGAGC | |
| gRNA sequence: 10aA2: | 25 |
| AGACGCTCAACTTTGCGCAG | |
| gRNA sequence: | 26 |
| Non-targeting Scramble: | |
| AAATGTGAGATCAGAGTAAT | |
| Primer: TCF4 3b Forward: | 27 |
| GTGCCGAAACTACACTTTTGTG | |
| Primer: TCF4 3b Reverse: | 28 |
| ACAAGATTATGCACCTGGCT | |
| Primer: TCF4 8a Forward: | 29 |
| TGGAAAGGGGATGCTTACTCTC | |
| Primer: TCF4 8a Reverse: | 30 |
| AACTCTAATGACCTCCGCCT | |
| Primer: TCF4 10a Forward: | 31 |
| TTACCCTAAGGCAGTTGAGTGGA | |
| Primer: TCF4 10a Reverse: | 32 |
| TTGTCAAAAATCCCCCTCGCA | |
| Primer: TCF4 4/5 Forward | 33 |
| (isoform TCF4-B): | |
| CCTCCTGTGAGCAGTGGGA | |
| Primer: TCF4 4/5 Reverse | 34 |
| (isoform TCF4-B): | |
| CTGGACGGGCTTGGATGTC | |
| Primer: TCF4 9/10 Forward | 35 |
| (isoform TCF4-D): | |
| GCCATCTTCAGTCTATGCTCCATC | |
| Primer: TCF4 9/10 Reverse | 36 |
| (isoform TCF4-D): | |
| TAGGGAAAGTGCTGGTTGCTGG | |
| Primer: TCF4-A Forward | 37 |
| (isoform TCF4-A): | |
| GGAAAGCGGTCTATGCTCCAT | |
| Primer: TCF4-A (9/10) Reverse | 38 |
| (isoform TCF4-A): | |
| TAGGGAAAGTGCTGGTTGCTGG | |
| Primer: TCF4 12/13 Forward | 39 |
| (all isoforms): | |
| CTTCCTCCGATGTCCACTTTCCA | |
| Primer: TCF4 12/13 Reverse | 40 |
| (all isoforms): | |
| CCCGCTTCCTCTATTTGCCAT | |
| Primer: MPH Forward: | 41 |
| ATCAGGGCGTGTCCATGTCTCA | |
| Primer: MPH Reverse: | 42 |
| CGGGTAATGGCTTCGGGGTA | |
| Primer: dCas9 Forward: | 43 |
| GCCGACGCTAATCTGGACAAAGT | |
| Primer: dCas9 Reverse: | 44 |
| TGGTCAGGGTAAACAGGTGGATG | |
| Primer: TBP Forward: | 45 |
| TGTATCCACAGTGAATCTTGGTTG | |
| Primer: TBP Reverse: | 46 |
| GGTTCGTGGCTCTCTTATCCTC | |
| Primer: CNTNAP2 Forward: | 47 |
| TCACACAGACCAAGATGAGCCAA | |
| Primer: CNTNAP2 Reverse: | 48 |
| TAGGAAGCGAACCTCGTGCCA | |
| Primer: KCNQ1 Forward: | 49 |
| GGACAAAGACAATGGGGTGACT | |
| Primer: KCNQ1 Reverse: | 50 |
| GTGTTGGGCTCTTCCTTACAGAA | |
| Primer: TCF4B_cDNA Forward: | 51 |
| ATGCATCACCAACAGCGAATGG | |
| Primer: TCF4B_cDNA Reverse: | 52 |
| ATCCTCGAGTTACATCTGTCCCA | |
| TGTGATTCG | |
| Primer: minP_TCF4: | 53 |
| GAATTCAGAGGGTATATAATGGA | |
| AGCTCGACT | |
| TCCAGATGCATCACCAACAGCGAATGG | |
| Primer: E-box-x6-minP_TCF4: | 54 |
| GAATTCAACACCTGCAAGAACACCTGC | |
| AAGAACACCTGCAAGAACACCTGCAA | |
| GAACACCTGCAAGAACACCTGCAAGA | |
| GAGGGTATATAATGGAAGCTCGACTT | |
| CCAGATGCATCACCAACAGCGAATGG | |
| Primer: E-box-x12-minP_TCF4: | 55 |
| GAATTCACCTGCAAGAACACCTGCAAG | |
| AACACCTGCAAGAACACCTGCAAGAA | |
| CACCTGCAAGAACACCTGCAAGAACA | |
| CCTGCAAGAACACCTGCAAGAACACC | |
| TGCAAGAACACCTGCAAGAACACCTG | |
| CAAGAACACCTGCAAGAGAGGGTATA | |
| TAATGGAAGCTCGACTTCCAGATGCA | |
| TCACCAACAGCGAATGG | |

For organoid transduction experiments (FIGS. 11F-H and 19M-S), lentiviral particles were prepared using the second-generation lentiviral production plasmids psPAX2 (Addgene #12260) and pMD2.G (Addgene #12259). Thirty 10 cm plates of 80% confluent HEK293T cells were transfected with 10 μg of plasmid per plate, the 2^{nd} Generation Packaging mix (ABM; LV003), and Lentifectin transfection reagent (ABM; G074), using the manufacturer's recommendations. Two days after transfection, the supernatant from all plates was harvested and the viruses were purified by PEG precipitation using PEG-it Virus Precipitation Solution (Systems Biosciences; LV810A-1). Titer determination was achieved using the qPCR Lentiviral Titration kit (ABM; LV900). All titers were above 10⁹ IU (particles) per μL. Equivalent AAV vectors were ordered from Applied Biological Materials (ABM) of AAV9 serotype with the human growth hormone (hGH) terminator in each construct. All titers were commercially determined as >10⁹ IU (particles) per μL. Transduction of organoids (CtOs) was achieved by mixing 3.5 million dissociated iPSCs on the first day of organoid derivation (lentiviruses; FIG. 11F) or by adding AAV virus directly to the medium after the last day of the neural induction phase (FIG. 11H) with the appropriate virus quantities to obtain a multiplicity of infection (MOI) of 5 for each type of vector.

CRISPR-mediated trans-epigenetic correction of TCF4 expression. First, RNA sequencing libraries were analyzed from PTHS and control NPCs to determine the transcriptional activity from the numerous alternative promoters of the human TCF4 gene (Sepp et al., 2011). Promoter activity estimation was performed using the junction read counts approach described in (Demircioglu et al., 2019). Briefly, exon junction counts were obtained by mapping the RNA-seq reads onto the GRCh38.p13 genome assembly with the STAR aligner (version STAR 2.7.6a), using the GENCODE 34 primary annotation as a reference to determine exon coordinates. Next, the proActiv R package (version 0.99.0) was used to estimate promoter activity by counting the junction reads mapping to the first set of introns of each TCF4 transcript, followed by normalization of the counts using the size factors approach and log-transformation of the data. This approach identified promoters upstream of exons 3b, 8a, and 10a as the most active in both parent and PTHS samples (FIG. 18A) and were therefore chosen for CRISPR-mediated trans-epigenetic manipulation of TCF4 transcriptional activity.

At these three promoters, gRNAs were designed based on sequences located between −100 and +50 from the corresponding transcriptional start sites (TSS) (Liao et al., 2017) (FIG. 18A). For each promoter, 3 sense and 2 antisense gRNAs were selected based on the score generated by the computational tool designed by (Hsu et al., 2013). As a control gRNA sequence, a non-targeting scrambled sequence was selected (see Table 2 for gRNA sequences). gRNAs were validated by first inserting the corresponding sequences into the traditional CRISPR pSpCas9(BB)-2A-Puro plasmid (Addgene #48139; [http:/]/n2t.net/addgene:48139; RRID:Addgene_48139), followed by testing the efficiency of each gRNA to generate indels in pilot experiments. To this end, pSpCas9(BB)-2A-Puro was digested with BpiI (Thermo Fisher Scientific). Each synthesized gRNA oligonucleotide pair was phosphorylated with T4 polynucleotide kinase (Promega) and annealed by incubation in a thermocycler under the following conditions: 30 min at 37° C., 5 min at 95° C., and ramp down to 25° C. at 5° C. min⁻¹. Phosphorylated oligonucleotide duplexes for each gRNA were then ligated to the digested plasmid by incubation at 25° C. for 1 h with T4 DNA ligase (Promega). Competent cells (Stbl3 E. coli bacterial strain; Thermo Fisher Scientific) were transformed with each ligation product and plasmid DNA was extracted from each clone with the PureYield Plasmid Miniprep System (Promega), followed by validation via Sanger sequencing with the hU6-F universal primer.

Next, HEK293T cells (ATCC) were cultured in DMEM containing 10% FBS and 1% penicillin/streptomycin to 70% confluency and transfected with each gRNA plasmid using polyethylenimine (PEI; Sigma-Aldrich) at a ratio of 3:1 PEI/DNA (w/w), with 1 μg of DNA per mL of culture medium. Both PEI and DNA were diluted in Opti-MEM (Gibco) at a volume 1/20 of the total volume of culture medium, followed by incubation for 30 min before being applied directly on top of the cells. Transfection medium was replaced with culture medium 16 h after transfection. To confirm that the selected TCF4 gRNA sequences indeed targeted TSS 3b, 8a and 10a of the human TCF4 gene, T7 endonuclease I assays were performed. Transformed cells were selected by replacing the transfection medium with culture medium supplemented with 1 μg mL⁻¹ puromycin until all cells in the negative control died (˜72 h). Genomic DNA was then extracted using the Illustra Blood Genomic Prep Minispin kit (GE), according to the manufacturer's instructions. One designed primer pair flanking each gRNA's target site in the genome was used and end-point PCR with Q5 high-fidelity DNA polymerase (NEB) was used. Amplicons were purified with the Wizard SV Gel and PCR Clean-Up System (Promega) and quantified using the Qubit DNA BR Assay Kit (Thermo Fisher Scientific). For the T7 endonuclease I assay, 300 ng of amplicons of each sample were incubated with 2 μL NEBuffer 2 and H₂O (to a final volume of 19.5 μL) in a thermocycler, with the following cycling parameters: 5 min at 95° C., ramp down to 85° C. at −2° C. min⁻¹, ramp down to 25° C. at −0.1° C. min⁻¹. After denaturation and gradual re-annealing to allow formation of DNA heteroduplexes, 5U of T7 endonuclease I (NEB) were added to the samples and incubated for 30 min at 37° C. The products were run on a 1.5% agarose gel and the CRISPR-mediated efficiency for creation of indels was estimated for each gRNA based on the ratio between the masses of undigested bands and digested fragments. Additionally, the amplicons were deep sequenced and the percentages of clones with indels were computed.

All gRNAs were also cloned into the pLentiSAMv2 plasmid (Addgene plasmid #75112; [http:/]/n2t.net/addgene:75112; RRID:Addgene_75112), harboring the gRNA sequence with MS2 loops at both the tetraloop and the stem loop 2 under the control of the U6 promoter, along with the dead Cas9 (dCas9) gene fused to the VP64 gene under the control of the EF1α promoter. Cloning was performed in pLentiSAMv2 via digestion with Esp3I (Thermo Fisher Scientific). Next, lentiviral particles were prepared by transfecting HEK293T cells (ATCC) with suitable pLentiSAMv2 vectors carrying the tested gRNAs or the scrambled gRNA (control).

To evaluate the efficiency of the designed TCF4 gRNA sequences at increasing endogenous expression of the TCF4 gene via trans-epigenetic activation, SH-SY5Y cells were transfected with the pLentiSAMv2 and pLentiMPHv2 (Addgene #89308; [http:/]/n2t.net/addgene:89308; RRID:Addgene_89308) plasmids, followed by RT-qPCR to verify the levels of TCF4 transcripts. The pLentiMPHv2 vector contains the MS2-P65-HSF1 activator helper (MPH) complex gene under the control of the EF1α promoter (Liao et al., 2017), in combination with the gRNA and dead Cas9, for the trans-epigenetic activation of the TCF4 locus.

SH-SY5Y cells were cultured in DMEM/F12 containing 10% FBS and 1% penicillin/streptomycin. SH-SY5Y cells were transfected with FuGENE HD Transfection Reagent (Promega) at a ratio of 4:1 FuGENE/DNA (v/w), with 2 μg of DNA per mL of culture medium. Both FuGENE and DNA were diluted in Opti-MEM (Gibco) at a volume 1/10 of the total volume of culture medium, without an incubation period before being applied to the cells. Transfection medium was replaced with culture medium supplemented with 10 μg mL⁻¹ blasticidine S (Sigma-Aldrich) 16 h later, for selection of transfected cells. After control cells died (˜72 h), selection medium was replaced with culture medium to allow cells to expand. For each gRNA, transfection was performed in triplicates. RNA from selected cells was purified with TRIzol reagent (Thermo Fisher Scientific), according to the manufacturer's instructions. cDNA was synthesized with ImProm-II Reverse Transcription System (Promega). For RT-qPCR reactions, primer pairs were designed to be able to detect (I) transcripts encoding TCF4-B, TCF4-D, or TCF4-A (depending on the corresponding promoter targeted by each gRNA), (II) transcripts of the endogenous TBP gene, and the exogenous dCas9 (encoded by lentiSAMv2) and MPH (encoded by lentiMPHv2) genes, and (III) transcripts from genes transcriptionally regulated by TCF4. All reactions were performed with technical duplicates, using the PowerUp SYBR Green Master Mix (Applied Biosystems) on a QuantStudio 6 Flex Real-Time PCR System (Applied Biosystems). A melt-curve step was always included at the end of each run. Samples transfected with an empty lentiSAMv2 plasmid or with a plasmid containing the scrambled gRNA were used as references for the quantification of relative levels of TCF4 and TCF4-target gene transcripts via the traditional ΔΔCt method, with transcript levels of TBP, dCas9 and MPH used for normalization between samples.

For organoid transduction experiments (FIGS. 11B-D and 18E-F), lentiviral particles were prepared from pLentiMPHv2 vector and the several pLentiSAMv2 versions containing different gRNAs (Liao et al., 2017). For virus preparation, the second-generation lentiviral production plasmids psPAX2 (Addgene #12260) and pMD2.G (Addgene #12259) were used. Twenty 10 cm plates of 80% confluent HEK293T cells were transfected with 10 μg of plasmid per plate, the 2^{nd} Generation Packaging mix (ABM; LV003), and Lentifectin transfection reagent (ABM; G074), using the manufacturer's recommendations. Two days after transfection, the supernatant from all plates was harvested and the viruses were purified by PEG precipitation using PEG-it Virus Precipitation Solution (Systems Biosciences; LV810A-1). Titer determination was achieved using the qPCR Lentiviral Titration kit (ABM; LV900). All titers were above 10⁹ IU (particles) per mL.

Transduction of organoids (sPOs) was achieved by mixing 2.5 million dissociated iPSCs on the first day of organoid derivation with the appropriate virus quantities to obtain a multiplicity of infection (MOI) of 5 for each type of lentivirus. Organoids in the ‘scrambled gRNA’ condition (control) were co-transduced with lentiviruses produced from pLentiMPHv2 and pLentiSAMv2 plasmids containing a scrambled gRNA. Organoids in the ‘TCF4 gRNA’ group were co-transduced with lentiviruses produced from pLentiMPHv2 and pLentiSAMv2 plasmids containing gRNA version 3bS3. On the second and third days of organoid derivation, medium was changed and lentiviruses were added again. During these 3 days, embryoid bodies were formed in the presence of mTeSR1 medium containing SB431542 and dorsomorphin. From the fourth day onward, medium was changed according to the regular protocol, without the addition of viruses. Transduction was confirmed by the evaluation of Cas9 expression via immunostaining, using the protocol described herein and above.

Statistical analyses. Data are presented as mean+SEM, unless otherwise indicated. Statistical methods such as power analysis to determine sample size were not performed, because of the limited PTHS samples available, which were chosen based on availability of detailed information about the types of TCF4 mutation carried by each patient. However, based on the strong and consistent effect sizes observed throughout the study and on the level of variability across cell lines from all subjects (in NPCs and organoids), increasing sample size is not expected to change statistical significance of the results.

Different types of statistical test were used throughout the study, as indicated in the corresponding figure descriptions. Usually, comparisons of means between two groups (PTHS against parent) in experiments that measured organoid size, relative expression levels, or expression abundances used two-sample Welch's t test, assuming unequal variances and heteroskedasticity. When comparing these types of means among more than two groups, a one-way Analysis of Variance (ANOVA) was used, followed by Tukey's Honestly Significantly Different (HSD) post-hoc test. For comparing mean gene expression in single cell RNA-Seq data between two samples, the non-parametric Mann-Whitney U-test was used. For the same type of comparison among more than two samples, the Kruskal-Wallis test was used, followed by Dunn's post-hoc test. For comparisons of mean gene expression values in single cell transcriptomic data, the calculated p-values are presented as asterisks in the figures but added a cross symbol (t) to those comparisons in which the fold change between PTHS and parents was smaller than an arbitrary value of 10% of the parent group mean, in either direction. For comparing neurite length and soma area between PTHS and control neurons, ANOVA with Geisser-Greenhouse correction for repeated measures was used, followed by Fisher's Least Significantly Different (LSD) post-hoc test.

Sample sizes are indicated in the figure legends. P-values are reported as asterisks in the figures for significance levels defined as p<0.05 (*), p<0.01 (**), or p<0.001 (***). When experimentation involved more than one independent replicate per subject cell line, or more than one technical replicate per independent replicate, the numbers of replicates are also indicated in the figure legends, even though each statistical test was computed based solely on the comparison between the means of different subjects. Blinding was used for most analyses comparing patients and control samples. Statistical analyses were performed using Prism software (GraphPad), RStudio, G*Power, and WebPower.

Brain cortical organoids derived from Pitt-Hopkins Syndrome patients exhibit aberrant size and morphology. To gain insight into the largely unknown cellular pathophysiology caused by mutations in TCF4, iPSC lines were generated via cellular reprogramming of skin fibroblasts from five PTHS patients and corresponding parents of matching sex (Table 1). These individuals harbor either mutations that eliminate the TCF4 gene partially or entirely, eliminate its essential bHLH DNA-binding domain, or impact one of its transcriptional activation domains (FIG. 12A). All iPSC clones were checked for the expression of stem cell markers, and SNP mapping-based karyotypic analysis revealed no unwanted chromosomal abnormalities (FIG. 12B). No differences between PTHS and control iPSC lines were observed in terms of growth rate (FIG. 12C) or general ability to derive NPCs and neurons in vitro (FIG. 12D-E).

Next, the iPSC lines were used to generate brain cortical organoids (CtO) (FIG. 5A), followed by evaluation of aberrant phenotypes at the cellular and molecular levels. CtOs transcriptionally resemble the human cortex during early development, include functional glutamatergic and GABAergic neurons, and exhibit cellular populations that functionally resemble features observed during human neurodevelopment. Control (parent) CtOs exhibited the expected three-dimensional organization into spheroids, which grew continuously in size and developed clearly visible rosette-like cellular aggregates (arrowhead in top row in FIG. 5A). In marked contrast, PTHS CtOs are smaller (FIGS. 5A-B) and harbor a noticeably fewer discernible rosettes and smaller size (FIG. 5A). Some PTHS organoids exhibit a polarized structure (arrowheads in bottom row in FIG. 5A). These phenotypes are consistent across batches performed with different clones derived from the same patient (FIG. 12F).

While the CtOs recapitulate several aspects of cortical development, they are mostly devoid of GABAergic cells of subpallial origin (FIG. 6D). Therefore, subpallial organoids (sPO, FIG. 5C), were also derived, which contain neural progenitor cells and GABAergic inhibitory neurons. Similar to what was observed in CtOs, PTHS sPOs also display smaller size (FIG. 5C) and aberrant internal organization, with few or absent rosettes (FIG. 12H).

Together, these results show that PTHS brain organoids exhibit aberrant morphology, suggesting that processes underlying neural development may be altered in PTHS patients. Moreover, the extent of the phenotypic differences between control and patient-derived brain organoids confirms that such human cellular models can be used to study PTHS pathophysiology and molecular mechanisms.

Abnormal content of progenitor cells and neurons in PTHS organoids. Smaller organoids may result from a range of altered cellular processes, such as decreased cell division or increased apoptosis, abnormal migration, or senescence. To identify which of these processes are defective in PTHS organoids, the organization and contents of several key cellular subtypes was analyzed. First, immunostaining for neural progenitor marker SOX2 was performed on histological sections from patient and control CtOs and sPOs. At 4 weeks in vitro, control CtOs contain a large number of rosettes composed of neural progenitors surrounding a ventricle-like lumen, similar to the distribution of ventricular and sub-ventricular zone progenitors in the developing human brain. As these progenitor-rich structures differentiate into several neuronal subtypes, the rosettes diminish in size. In contrast to control CtOs, PTHS organoids display very few rosette-like structures, and neural progenitors are dispersed throughout the organoid without any apparent organized clustering (see supporting controls in FIG. 12F). Moreover, most PTHS CtOs are polarized, with SOX2-positive cells concentrated on one side. It was found that PTHS CtOs have a significantly lower density, but higher percentage of neural progenitors compared to control organoids (FIGS. 5D and 12F), in keeping with fewer rosette structures where progenitors tend to localize. Likewise, PTHS sPOs display a reduction in the content of SOX2+ progenitors (FIGS. 12H-I).

Immunostaining for neuronal marker MAP2 revealed that control CtOs have neurons dispersed throughout the spheroid, particularly around and between rosettes, and neuronal content increases as parent CtO development proceeds. Dissimilarly, PTHS CtOs and sPOs possess less evident MAP2 labeling, even in later stages of organoid development (FIG. 12H). Parental CtOs exhibit a typical pattern of cortical development, recapitulating the temporal progression of neuronal differentiation in the human cortex, in which deep-layer neurons (i.e., CTIP2+ cells) form first, followed by differentiation of superficial layer neurons (SATB2+ and CUX1+ cells) (FIG. 12J). In contrast, PTHS CtOs exhibit a severely reduced content of cortical neuron subtypes (FIGS. 5E and 12J). Additionally, mature PTHS CtOs display a reduction in staining for vesicular glutamate transporter family member 1 (vGLUT1) (FIG. 12M), a marker of excitatory neurons previously shown to be abundant in CtOs. Similarly, PTHS sPOs have reduced staining for GABAergic neuron markers GAD65/67 (FIG. 12H). Importantly, similarly decreased expression of MAP2 and of cortical neuron markers (FIG. 12K) as well as lower numbers of cortical-type neurons (FIG. 12L) was observed in a post-mortem PTHS brain sample.

Together, these data indicate that the patient-derived organoids closely match the neural phenotypes observed in the patient at the cellular level and confirm that PTHS is characterized by severe deficits in cortical neuron content and organization.

PTHS organoids exhibit lower percentages of neurons and higher percentages of progenitors. To better quantify the cellular diversity of brain organoids, single-cell RNA sequencing (scRNA-Seq) was performed on dissociated cells from CtOs and sPOs. Six annotated cellular subpopulations were analyzed—neural progenitors, intermediate progenitors, and mature neurons in both CtOs and sPOs (FIGS. 6A, 13A-C). These groups of cells were further analyzed because differentiation trajectory analysis indicated that they compose two distinct differentiation lineages, in which neural progenitors progress through an intermediate progenitor stage towards the generation of glutamatergic neurons (excitatory lineage) or GABAergic neurons (inhibitory lineage) (FIG. 6B). Therefore, studying these populations is appropriate for assessing deficits in neural development across the spectrum of cortical cell types. The organoids do not contain cells expressing mesoderm and endoderm markers (FIG. 12C), and other smaller populations were not studied (‘Others’ in FIG. 13A) because, even though they are of neural origin (FIG. 13E), they could not be unequivocally assigned to the six populations chosen for analysis.

PTHS organoids have reduced density of progenitors per area (FIG. 5D), but scRNA-Seq data and immunostaining revealed that the percentage of progenitors is higher in PTHS CtOs relative to control organoids (FIGS. 6C, 6D, 6E and 12G). Similarly, PTHS sPOs possess higher percentages of subpallial progenitors than control sPOs (FIGS. 6F, 6G and 6H). Moreover, astrocytic content is small and similar in PTHS and control organoids (FIG. 13F), ruling astroglia content out as a potential cause for phenotypic abnormalities in PTHS organoids.

In agreement with the finding that PTHS organoids have lower neuronal content (FIGS. 5D, 5E), scRNA-Seq analysis showed a decrease in the percentages of excitatory and inhibitory neurons in PTHS CtOs and sPOs, respectively, as compared to control organoids (FIGS. 6C, 6D, 6F and 6G). Additionally, the percentages of neurons expressing BCL11B (coding for CTIP2), SATB2, TBR1, and CUX1 are lower in PTHS CtOs (FIGS. 6I, 13H and 13I), as is the number of neurons expressing GAD2 (coding for GAD67) in PTHS sPOs (FIG. 6J).

Together, these data indicate that PTHS organoids have proportionally more progenitors and fewer neurons, suggesting that the disease's pathophysiology includes defects in progenitor proliferation and/or differentiation into neurons.

PTHS neurons exhibit abnormal firing properties. The diminished neuronal content in PTHS organoids suggests that the formation of neuronal circuits may be impaired in the patients' neural tissue. Moreover, reduced staining for vGLUT1 in PTHS CtOs may indicate that the mutant neurons establish fewer synapses and exhibit impaired electrical activity. To investigate these issues, PTHS neurons in 2D culture and organoids were used. First, multi-electrode array (MEA) assays were used to analyze neuronal activity and found that mean neuronal firing rates were much lower in PTHS as compared to control CtOs (FIGS. 7A and 14A). PTHS CtOs contain a substantial amount of neurons (FIGS. 5D and 5E), therefore such diminishment in electrical activity likely reflects electrophysiological defects at the cellular level. However, neuronal content is impaired in PTHS CtOs (FIGS. 5E, 61, 12J, 13H and 13I), thus it is formally possible that the PTHS electrical activity defects are the result of poor connectivity or neuronal density in the organoids.

To assess the impact of TCF4 haploinsufficiency on individual neurons and determine whether the diminished activity in organoids might stem from aberrant electrical neuronal properties, 2D neuronal cultures were studied. First, experiments were performed to confirm that TCF4 is expressed in control neurons (FIG. 14B) and that PTHS neurons have a reduction in TCF4 expression compared to the respective parental controls (FIG. 14C). Through analysis of neuronal arborization architecture (FIG. 7B), it was concluded that the soma area of PTHS neurons was indistinguishable from that of parental controls, while neuronal processes were longer in PTHS neurons (FIG. 7C). Next, a patch-clamp analysis was performed to assess neurons in 2D culture from the most significantly impaired patient line (FIG. 7A). It was found that PTHS neurons exhibited severely decreased intrinsic excitability (FIG. 7D), membrane capacitance, and sodium and potassium currents (FIGS. 7E, 14D and 14E). Furthermore, lower expression of the surrogate marker of neuronal activity FOS was observed in neurons in PTHS CtOs as compared to control CtOs (FIG. 7F). In combination, these data show that PTHS neurons exhibit severe deficits in electrical properties at the network and cellular levels.

Such neuronal dysfunctions may arise from abnormal gene expression in PTHS neurons, and therefore RNA sequencing was used to probe transcriptomic alterations in these cells, comparing neurons differentiated from iPSC-derived patient and control neural progenitors under 2D culturing conditions. Differential expression (DE) analysis comparing PTHS and control neurons from 2-months-old FACS-sorted cultures revealed a range of mis-regulated genes (FIG. 14F), several of which are involved in neurogenesis, neuronal identity, differentiation, and regulation of neuronal excitability (FIG. 14G). Among those with higher fold-changes (above 4), there are several genes important in neuronal function (FIG. 7G), which are also downregulated in neurons from PTHS organoids (FIG. 7H).

Together, these data indicate that neurons derived from PTHS patients are aberrant in terms of morphology, physiology, and transcriptomic landscape. Importantly, the list of DE genes includes potassium voltage-gated channel subfamily Q member 1 (KCNQ1), previously shown to dysregulate intrinsic excitability of mouse neurons after Tcf4 knockdown (Rannals et al., 2016), as well as a number of other ion channels (FIG. 14H), which not only offer mechanistic insight into the PTHS neuronal intrinsic excitability defects but also provide new opportunities for pharmacological therapeutic intervention.

PTHS neural progenitors display lower proliferation rates and replicative senescence. The finding that PTHS CtOs and sPOs have fewer neural rosettes and lower density (but higher percentage) of NPCs leads to the question of whether these phenotypes are the result of abnormal neural induction, reduced progenitor proliferation, or impaired differentiation. To assess these possibilities, the number of rosettes was counted after the neural induction phase (week 2) and showed that it is similar in parent controls and PTHS organoids, as is the density of SOX2+ cells (FIG. 8A). These results, together with the absence of cells expressing non-neural markers in organoids (FIGS. 13D and 13E), strongly suggest that neural induction is normal in PTHS organoids and that rosettes dwindle at later stages during organoid maturation either due to poor progenitor proliferation or impaired differentiation. To parse out between these two possibilities, iPSC-derived NPCs were produced from PTHS individuals and parental controls (see expression of NPC markers in FIG. 15A). These cells indeed express TCF4 (FIGS. 15B, 15C and 15D), and its expression is reduced in PTHS NPCs (FIGS. 15E and 15G). Importantly, the expression of GADD45G, a direct transcriptional target of TCF4, is strongly reduced in PTHS NPCs (FIG. 15F), confirming that TCF4 function is significantly impaired in all patient lines. It was observed that PTHS NPCs grow significantly slower in 2D culture than control lines (FIGS. 8B and 8D). As this difference may arise from either decreased proliferation or increased apoptosis, Annexin V-mediated flow cytometry was used to assess the apoptotic rate, and concluded that PTHS and parental NPCs do not notably differ in the percentage of apoptotic cells, which is generally less than 5% (FIG. 8C).

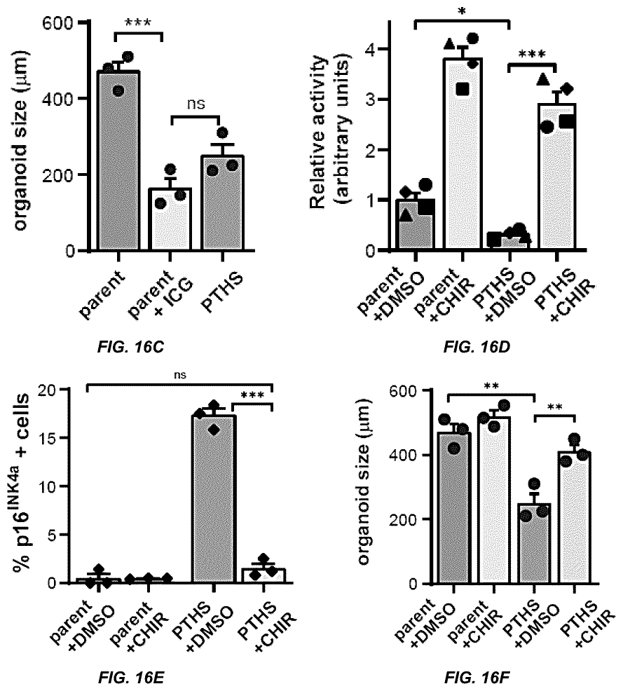

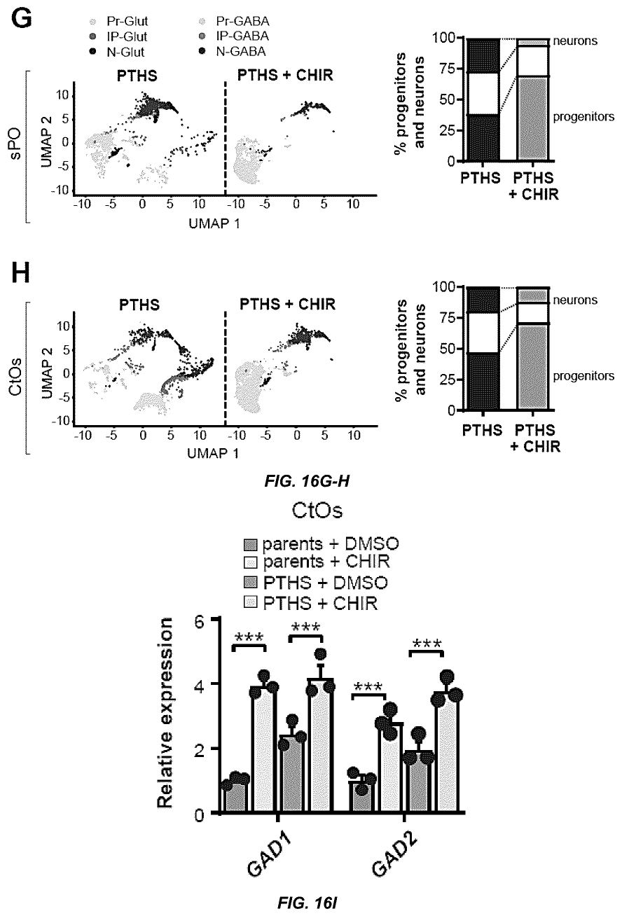

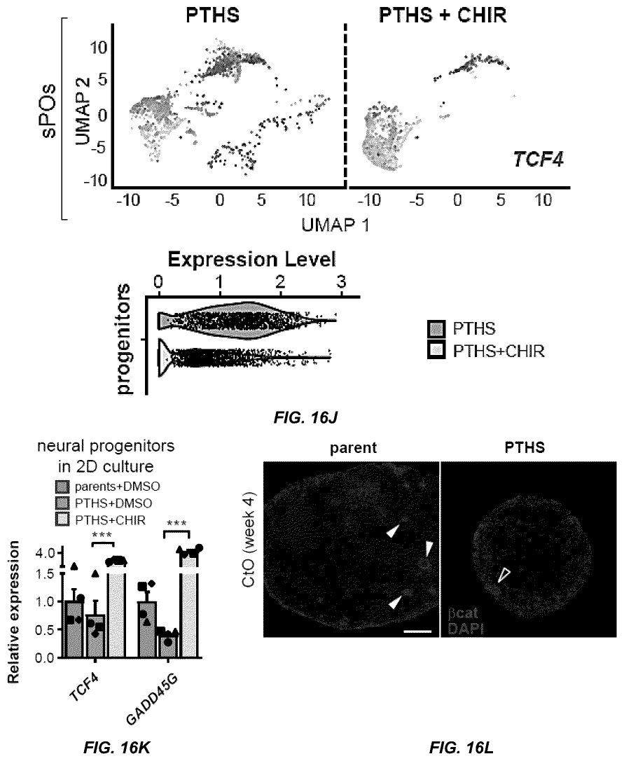

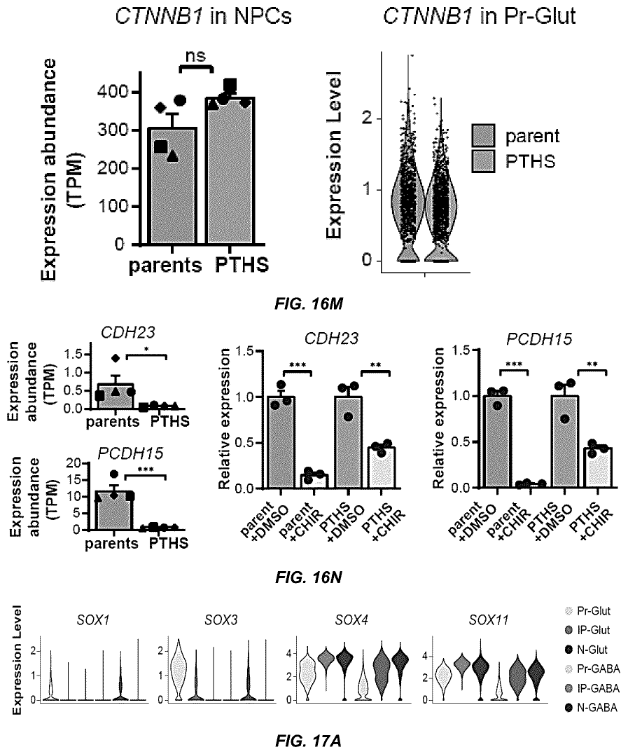

Next, experiments were performed to assess the proliferative capacity of NPCs by incubating the cell cultures with 5-ethynyl-2′-deoxyuridine (EdU), a thymine nucleoside analog which is incorporated into DNA during synthesis, followed by flow cytometry-based determination of the percentage of cells undergoing division. PTHS lines exhibit approximately half the percentage of proliferative cells than control NPCs (FIG. 8D). It was also observed that PTHS NPCs frequently assume an atypical enlarged, flat morphology (arrowheads in FIG. 8G), which was not observed in the control lines. The combination of aberrant morphology and diminished proliferative activity led to the hypothesis that PTHS neural progenitors are undergoing precocious replicative senescence. This is a well-defined cellular process characterized by cell cycle arrest and subsequent halting of proliferation and shown to participate in a variety of physiological and pathological defects. In fact, three hallmarks indicative of replicative senescence were observed in PTHS NPCs in addition to larger cell size: heightened β-galactosidase activity (SA-β-gal; FIG. 8G), reduced expression of the nuclear lamina protein lamin B (LMNB1; FIG. 8H), and markedly increased expression of cyclin-dependent kinase inhibitor genes CDKN2A (FIG. 8H) and CDKNIA—whose expression causes cell division to stall and acts as markers of replicative senescence. Interestingly, these characteristics intensify with increasing number of passages (FIG. 15H). Moreover, the senescent NPCs are Nestin+, most are SOX2+; SOX2 expression fades in some NPCs, and are negative for Brachyury and SOX17 (FIG. 16E), indicating that they are senescent NPCs and not mis-differentiated cells.

Interestingly, the expression of senescence markers is also strongly upregulated in the PTHS post-mortem sample (FIG. 8I). PTHS CtOs contain many neural lineage cells expressing the CDKN2A gene product, p16^{INK4A} (FIGS. 8J and 15J) and these are not apoptotic cells, which are similarly infrequent in both PTHS and control organoids (FIG. 8J). Importantly, shRNA-mediated TCF4 knockdown in control NPCs led to decreased proliferation (FIG. 15K) and higher expression of CDKN2A (FIG. 15L), further strengthening the link between reduction in TCF4 expression and increased senescence and decreased proliferation in patient-derived NPCs.

Together, these data indicate that the pathological mechanism of PTHS at the cellular level involves decreased proliferation and augmented senescence of NPCs.

Correction of Wnt signaling in PTHS neural progenitor cells and organoids rescues aberrant phenotypes. To gain mechanistic insights into the aberrant proliferative activity of PTHS NPCs, an unbiased investigation of differentially expressed (DE) genes was examined between PTHS and control cells from 4 parent-child pairs (FIG. 15M), followed by gene set enrichment analysis (FIG. 15N). This approach revealed alterations mostly in the expression of genes in the Wnt signaling pathways (FIGS. 9A and 16A). Because the Wnt pathway has been linked to NPC proliferation in many tissues, the hypothesis was raised that abnormal Wnt activity may be causally implicated with the lower NPC proliferation rates observed in PTHS cells. To test this hypothesis, the expression of Wnt components in PTHS NPCs were analyzed to confirm that WNT2B, WNT3, WNT5A, and SFRP2 have lower expression in patient lines (FIGS. 9A-B), and functional assessment of signaling using a luciferase reporter indicated prominent reduction in canonical Wnt/β-catenin signaling activity (FIG. 9C). Importantly, expression of several Wnt signaling components is markedly downregulated in the post-mortem PTHS brain sample (FIG. 9D).

Treatment of control NPCs with Wnt signaling antagonists DKK-1 and ICG-001 phenocopied the reduction in PTHS progenitor proliferation rate (FIG. 9E) and the increase in CDKN2A expression (FIG. 16B). Treatment of control CtOs with ICG-001, which is a diffusible small molecule that can easily penetrate the three-dimensional organoid structure, led to marked reduction in organoid size (FIG. 16C) and content of SOX2+ cells (FIG. 9F). As a reverse approach, PTHS NPCs were treated with the Wnt signaling agonist CHIR99021. First, it was confirmed that Wnt signaling was increased in the treated cells (FIG. 16D).

Treatment with CHIR99021 rescued the proliferation rates of PTHS NPCs (FIGS. 9G-H), decreased the percentage of p16^{INK4a} senescent cells (FIG. 16E), and increased the expression of pro-proliferative gene HES1 as well as proneural genes ASCL1 and NEUROD1 (FIG. 9J). Treatment of PTHS CtOs caused a significant increase in organoid size (FIG. 16F) and NPC content (FIG. 9K), with the reappearance of conspicuous neural rosettes. Analysis of cellular diversity in CHIR99021-treated PTHS CtOs and sPOs confirmed an increase in the progenitor population (FIGS. 16G-H).

Treated CtOs possess an increased percentage of subpallial-type neural progenitors (FIG. 16H) and the expression of subpallial markers is higher in both parent and PTHS CHIR99021-treated organoids (FIG. 16I), raising the possibility that such partial cellular fate change, from a cortico-pallial to a subpallial trajectory, might have caused the reversal in cellular phenotypes observed in PTHS cells after Wnt activation. However, CHIR treatment does not lead to an increase in either size (FIG. 16F) or progenitor content (FIG. 9K) in parent organoids, and CHIR treatment does not lead to increased proliferation (FIGS. 9G-H) or to decreased percentage of senescent cells (FIG. 91) in parent NPCs in 2D culture. These results rule out fate restriction as the cause for phenotypic correction after Wnt signaling agonistic activation in PTHS cells.

CHIR treatment increased the expression of TCF4 and TCF4 downstream targets in PTHS NPCs in 2D culture (FIG. 16K), an effect previously reported upon exposure to high CHIR concentrations in other cell types, raising the possibility that the phenotypic correction after Wnt activation is due to increased TCF4. However, neural progenitors in the organoid's 3D structure do not exhibit an increase in TCF4 levels after CHIR treatment (FIG. 16J), allowing us to conclude that the rescue of proliferation defect in PTHS organoids after Wnt signaling activation was not due to increased TCF4 expression itself.

The smaller number of neural rosettes in PTHS organoids (FIG. 9D) suggests that neuroepithelial architecture of progenitors is defective. Since β-catenin is a key component of the Wnt signaling pathway and an important regulator of epithelial cell adhesion and integrity, a plausible hypothesis is that the diminished Wnt signaling in PTHS NPCs results in dysregulated β-catenin expression, leading to dismantling of rosettes and failure to organize the neural progenitors. In fact, even though the levels of expression of β-catenin in PTHS NPCs and in PTHS CtO progenitors remain unchanged (FIG. 16M), β-catenin expression is disorganized in PTHS organoids (FIG. 16L), strengthening the possibility that Wnt signaling downregulation leads to neuroepithelial integrity defects during neurodevelopment in PTHS.

The other GO category of DE genes in PTHS NPCs is ‘cadherin’ (FIG. 15N), therefore experiments were performed to determine whether the expression of cadherins or protocadherins is altered in PTHS cells. Most DE genes in this category are actually Wnt pathway components, except CDH23 and PCDH15. CDH23's expression is negligible (FIG. 16N), so this gene was discarded as a potential mechanistic candidate. PCDH15 is significantly downregulated in PTHS NPCs, however CHIR99021 treatment in NPCs further reduces its expression (FIG. 16N) in the same condition in which the cellular phenotypes are corrected (FIGS. 9G-I), ruling out PCDH15 as a plausible cause for the abnormal phenotypes in PTHS NPCs.

Together, the experiments confirm the mechanistic involvement of Wnt signaling in PTHS NPC proliferation defects. More importantly, these results demonstrate that the aberrant phenotypes described in PTHS NPCs and organoids can be pharmacologically corrected, an observation that may direct future efforts to treat diseases caused by TCF4 haploinsufficiency.

Mechanistic involvement of SOX genes in altered PTHS NPC proliferation and differentiation. Because PTHS organoids contain a higher percentage of NPCs, fewer neurons, and altered Wnt signaling compared to parental organoids (FIGS. 5 and 9), experiments were performed to define mechanistic players downstream of TCF4 and the Wnt pathway that could control NPC proliferation and differentiation. Because an interplay has been described between SRY-related HMG-box (SOX) proteins and Wnt signaling and due to their known roles in cell proliferation/differentiation, the expression of all SOX genes in PTHS NPCs was investigated. The results showed that SOX1, SOX2, SOX3 and SOX4 were significantly downregulated in patient-derived cells (FIG. 10A). Members of the SOXB subfamily (SOX1, SOX2, and SOX3) are traditionally regarded as regulators of cell proliferation. In fact, these genes were found to be predominantly expressed in progenitors and intermediate progenitors of CtOs and sPOs (FIGS. 13B, 17A-C). SOX1 is not substantially expressed in organoids (FIG. 17A), therefore the experimental efforts focused on SOX3; moreover, all PTHS lines exhibit decreased expression for this gene, quite substantially in some patients (FIGS. 10A-B).

First, experiments were performed to determine whether SOX3 is functionally downstream to TCF4, as shRNA-mediated TCF4 knockdown in control NPCs led to a decrease in SOX3 expression (FIG. 10C). An investigation of the post-mortem PTHS cortical sample indicated that both the expression of SOX3 (FIG. 10D) and the number of SOX3+ cells (FIG. 10E) are severely impaired. It was also verified that SOX3 is downstream of the Wnt signaling pathway because treatment of PTHS progenitors with the Wnt agonist CHIR99021 increased SOX3 expression (FIG. 10F). Importantly, shRNA-mediated SOX3 knockdown in control NPCs led to a reduction in cell counts (FIGS. 10G and 17D), increase in the expression of cell-cycle arrest gene CDKN2A (senescence marker) (FIG. 17E), and decrease in the expression of pro-proliferative gene HES1 and proneural gene ASCL1 (FIG. 17E), matching the phenotypes found in PTHS NPCs. Interestingly, transfection-mediated transient SOX3 over-expression did not lead to rescue of the proliferative defect in PTHS NPCs (FIGS. 17F-G). This could be due to lack of sustained SOX3 over-expression during the many days of the NPC proliferation assay or to the existence of other parallel dysregulated pathways.

The NPC differentiation rate is lower in PTHS, as judged by the neuron-to-progenitor ratio in differentiating 2D cultures (FIGS. 10J and 17J). Moreover, intermediate progenitors are scarcer in PTHS as compared to control CtOs and sPOs (FIG. 17K), and cells expressing intermediate progenitor marker POU3F2 (coding for BRN2) are less numerous in PTHS organoids (FIG. 17L). In combination, these results indicate aberrant differentiation of progenitors into neurons in PTHS neural tissue. Given the known role of members of the SOXC subfamily of SOX transcription factors (SOX4 and SOX11) as pro-differentiation factors during neurogenesis, a plausible hypothesis is that the PTHS differentiation abnormality is due to lower SOXC expression. In fact, these transcription factor genes are expressed in intermediate progenitors and neurons of CtOs and sPOs (FIGS. 10I, 17H-I). SOX4 is involved in the generation of intermediate progenitors and in their differentiation into early-born (CTIP2- and TBR1-positive) and late-born (BRN2-, SATB2, and CUX1-positive) cortical neurons. It was confirmed that SOX4 expression is lower in three of the PTHS progenitor lines (FIG. 10H), in intermediate progenitors and neurons of the excitatory lineage in PTHS CtOs (FIG. 10I), and in GABAergic neurons of PTHS sPOs (FIG. 17H). To test the involvement of SOX4 in the cell differentiation pathology, a locked nucleic acid antisense oligonucleotide (LNA ASO)-mediated SOX4 knockdown was performed in differentiating neuronal 2D cultures from two parent lines. Both cell count and transcriptomic analyses revealed that the differentiation rate (MAP2-to-SOX2 ratio) is reduced after SOX4 knockdown (FIG. 10L), mimicking the aberrant phenotypes of PTHS neuronal cultures (FIG. 10J).

One model is that TCF4 loss-of-function results in Wnt downregulation and, consequentially, in reduced SOX3 expression, leading to diminished proliferation and increased cellular senescence. In parallel, reduced SOX4 expression would lead to impaired differentiation in PTHS neural tissue, thereby contributing to the pathological phenotypes observed in the patient-derived cells.

Reversal of aberrant PTHS phenotypes via genetic correction of TCF4 expression. Experiments were perform to genetically manipulate TCF4 itself. First, TCF4 expression was corrected in PTHS organoids using a CRISPR-based trans-epigenetic strategy (Liao et al., 2017). In this method, two viral vectors are used to deliver three expression cassettes to target cells: one encodes a short guide-RNA (gRNA) coupled with an engineered RNA hairpin aptamer; the second encodes a transcriptional activation complex (MPH) that binds the aptamer; and the third encodes dead Cas9. Expression of these cassettes in target cells is expected to epigenetically transactivate the endogenous TCF4 locus, because Cas9-mediated gRNA binding to the TCF4 promoter congregates MPH, enhancing transcription from the downstream gene (FIG. 11A).

A collection of expression cassettes containing 15 different gRNAs targeting three alternative promoters of the TCF4 gene (upstream of exons 3b, 8a and 10a) were created. These promoters, which give rise to transcripts encoding TCF4 protein isoforms B, D, and A, respectively, were most active in both PTHS and parental control samples (FIG. 18A). Some gRNAs were found to efficiently transactivate TCF4 and its target genes in the neuronal cell line SH-SY5Y and some gRNAs ideally provided a 2-fold TCF4 expression increment (FIGS. 18B-C). Next, expression cassettes containing the most efficient gRNA were transduced into PTHS organoids derived from patient #4 and its respective parent control (FIG. 11B). This patient line was chosen because it shows the largest differences in terms of organoid size and cellular content compared to the respective control (FIGS. 5B and 5D), allowing for the identification of the benefits of TCF4 correction more easily. TCF4 correction was verified by an increase of TCF4 immunolabeling intensity (FIG. 11C) and of TCF4 mRNA levels (FIG. 18D). TCF4 expression in PTHS line #4 is the same as in the parental control line (FIG. 15E and upper panel in 18D), because patient #4 has a point mutation not expected to decrease transcript levels (FIG. 12A). The CRISPR-mediated correction strategy enhances both the endogenous and the mutated alleles (FIG. 18D, lower panel), and the globally increased TCF4 levels are accompanied by correction of GADD45G (FIG. 18E), a TCF4 downstream target, revealing functional correction of the TCF4 locus.

Organoids transduced with the TCF4 gRNA vector display a decrease in the expression of senescence gene CDKN2A, and a correction in the expression of neuronal marker MAP2 (FIG. 18E). Also, SOX3 expression increased in these organoids (FIG. 18E), even though the correction was partial, probably because not all cells in the organoids express SOX3. The PTHS histological phenotypic abnormalities were rescued in organoids subjected to TCF4 correction (FIG. 11D), yielding normal spheroids devoid of aberrant outgrowths (arrowheads in middle panel). SOX2 and MAP2 staining of transduced organoids indicated that the correction in morphology was accompanied by reestablishment of the organoid's internal architecture, with progenitors forming neural rosettes surrounding a lumen (arrowhead in right panel; FIG. 11D). The outgrowths in PTHS organoids usually contain aggregates of MAP2+ cells (insets in FIG. 11J), an abnormal feature that disappeared in the organoids after TCF4 correction upon transduction with the TCF4 gRNA.

Additionally, the presence of immature neurons in PTHS organoids that might indicate alterations in the formation of cortical neurons were investigated. DCX (doublecortin) was used as a marker of immature neurons and verified that, even though DCX+ cells are found in both parent and PTHS organoids, these cells sometimes form bundles of aberrantly shaped DCX fibers of high caliber in the outgrowths of PTHS organoids, a feature that was fixed after correction of TCF4 expression. Interestingly, DCX expression is lower in the PTHS post-mortem cortical tissue sample (FIG. 18G), in intermediate progenitors and neurons in PTHS CtOs and sPOs (FIG. 18H), and in PTHS neurons in 2D culture (FIG. 18I). Notably, DCX expression was corrected in the organoids transduced with TCF4 gRNA (FIG. 18F), further suggesting that the number of immature neurons returns to normal after CRISPR-mediated correction of TCF4 levels.

The CRISPR strategy described above requires the use of two viral vectors, which must be expressed at optimal levels to promote correction of TCF4 expression. As an alternative, a simpler procedure for correcting TCF4 levels was adopted in which the cells and organoids are subjected to over-expression (OE) of an extra-copy of the TCF4 gene via lentivirus or AAV transduction. In these vectors, the TCF4-B coding sequence was placed under the control of TCF4 binding motifs (pE5 boxes) (FIGS. 11E and 18J), an approach expected to prevent ectopic TCF4 expression. First, TCF4 and GADD45G expression is corrected in transduced PTHS NPCs (FIG. 18J), indicating that the strategy can be used for TCF4 genetic correction. Next, transduction with lentiviral TCF4 OE constructs were observed at the beginning of the organoid derivation protocol and led to increased intensity of TCF4 labeling and corrected TCF4 and CDKN2A levels (FIG. 18K) in organoids. After TCF4 OE, mature PTHS organoids exhibit abundant neural rosettes (FIG. 11E), along with corrected general morphology and rescued numbers of SOX2+ progenitor and CTIP2+ cortical neurons (FIGS. 11E and 18K). PTHS organoids subjected to TCF4 OE these effects are accompanied by a significant improvement in two key electrophysiological parameters—mean firing rate and number of network electrical bursts (FIG. 11G), a clear indication of functional rescue in the corrected organoids.

When TCF4 OE was performed after the neural induction phase using AAV vectors (FIG. 11H), a clear increase in TCF4 labeling intensity was achieved (FIG. 11H), as well as corrected TCF4 and CDKN2A expression, numbers of SOX2+ and CTIP2+ cells (FIG. 18N), and reappearance of abundant rosettes (FIG. 11H). This experiment not only shows that PTHS cellular pathology can be reversed but also indicates that TCF4 haploinsufficiency does not result in impaired neural induction, in keeping with the presence of rosettes in early stages of PTHS organoid development (FIG. 8A), strengthening the hypothesis that the cellular pathophysiology involves defects in progenitor proliferation.

These experiments lay out definitive evidence that the pathology observed in PTHS organoids is a consequence of diminished TCF4 expression. Importantly, the data provide proof-of-concept that the pathophysiology caused by TCF4 haploinsufficiency, including impaired progenitor proliferation and neuronal differentiation as well as dysregulated cellular senescence and expression of SOX genes, can be corrected at the cellular and tissue levels, paving the road for much needed treatments for this condition.

A number of embodiments have been described. Nevertheless, it will be understood that various modifications may be made without departing from the spirit and scope of the description. Accordingly, other embodiments are within the scope of the following claims.

1. A recombinant nucleic acid construct comprising a mini-promoter operably linked to a coding sequence for a TCF4 polypeptide.

2. The recombinant nucleic acid of claim 1, further comprising one or more transcription factor binding motifs.

3. The recombinant nucleic acid of claim 2, wherein the one or more transcription factor binding motifs are microE5 motifs.

4. The recombinant nucleic acid of claim 3, wherein the recombinant nucleic acid comprises from 1 to 15 microE5 motifs.

5. The recombinant nucleic acid of claim 4, wherein the recombinant nucleic acid comprises at least 5 microE5 motifs.

6. The recombinant nucleic acid of claim 4, wherein the recombinant nucleic acid comprises at least 10 microE5 motifs.

7. The recombinant nucleic acid of claim 4, wherein the recombinant nucleic acid comprises 12 microE5 motifs.

8. The recombinant nucleic acid of claim 4, having a general structure of: microE5ₙ-minipromoter-TCF4 coding sequence, wherein n is an integer in the range of 5 to 15.

9. The recombinant nucleic acid of claim 4, wherein the microE5 motif comprises the nucleotide sequence of SEQ ID NO:10.

10. The recombinant nucleic acid of claim 1, wherein the TCF4 polypeptide is TCF4-B.

11-24. (canceled)

25. A vector comprising a recombinant nucleic acid of claim 1.

26. The vector of claim 25, wherein the vector is a viral vector.

27. (canceled)

28. The vector of claim 25, wherein the vector is an adeno-associated virus (AAV) vector, lentiviral vector or gamma-retroviral vector.

29. The vector of claim 28, wherein the vector is an AAV9 vector.

30. A recombinant cell comprising the recombinant nucleic acid of claim 1.

31. A pharmaceutical composition comprising a vector of claim 25.

32. A method of treating a neurological or neurodevelopmental disease or disorder related in a subject, comprising transforming a neuron of the subject with a recombinant nucleic acid of claim 1.

33. The method of claim 32, wherein the neurological or neurodevelopmental disease or disorder is Pitt-Hopkins Syndrome, schizophrenia, autism, autism spectrum disorder, or 18q syndrome.

34. The method of claim 32, wherein the neurological or neurodevelopmental disease or disorder is Pitt-Hopkins Syndrome, and is associated with TCF4 haploinsufficiency.

35-45. (canceled)

46. A method of treating a neurological or neurodevelopmental disease or disorder related to TCF4 haploinsuffiency in a subject, comprising increasing expression of one or more of SOX3 and SOX4 in neurons of said subject.

47-74. (canceled)
